(19) United States
(12) Patent Application Publication
(10) Pub. No.: US 2023/0084762 A1
(43) Pub. Date: Mar. 16, 2023

(54) NOVEL CRISPR-CAS SYSTEMS FOR GENOME EDITING

(71) Applicant: PIONEER HI-BRED INTERNATIONAL, INC., JOHNSTON, IA (US)

(72) Inventors: GIEDRIUS GASIUNAS, VILNIUS (LT); ZHENGLIN HOU, ANKENY, IA (US); TOMAS URBAITIS, VILNIUS (LT); JOSHUA K YOUNG, JOHNSTON, IA (US)

(73) Assignee: PIONEER HI-BRED INTERNATIONAL, INC., JOHNSTON, IA (US)

(21) Appl. No.: 17/904,736

(22) PCT Filed: Feb. 11, 2021
(86) PCT No.: PCT/US21/17593

§ 371 Date: Aug. 22, 2022

Related U.S. Application Data
(60) Provisional application No. 62/980,750, filed on Feb. 24, 2020.
(60) Provisional application No. 63/030,964, filed on May 28, 2020.

(51) Int. Cl. C12N 9/22; C12N 15/11; C12N 15/82; C12N 9/78; C12N 15/90

(52) U.S. Cl. CPC C12N 9/22; C12N 15/11; C12N 15/8213; C12N 9/78; C12N 15/902; C12N 15/907; C12N 2310/20; C07K 2319/00

(57) ABSTRACT

Compositions and methods are provided for genome modification of a target sequence in the genome of a cell, using a novel Cas endonuclease. The methods and compositions employ a guide polynucleotide/endonuclease system to provide an effective system for modifying or altering target sequences within the genome of a cell or organism. Also provided are novel effectors and endonuclease systems and elements comprising such systems, such as guide polynucleotide/endonuclease systems comprising an endonuclease. Compositions and methods are also provided for guide polynucleotide/endonuclease systems comprising at least one endonuclease, optionally covalently or non-covalently linked to, or assembled with, at least one additional protein subunit, and for compositions and methods for direct delivery of endonucleases as ribonucleotide proteins.

### CROSS-REFERENCE TO RELATED APPLICATIONS

This application claims the benefit of U.S. Provisional Patent Application Nos. 62/980750 filed 24 Feb. 2020 and 63/030964 filed 28 May 2020, each of which is herein incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 8207-WO-PCT_SequenceListing_ST25.txt created on 4 Feb. 2021 and having a size of 158,138 bytes and is filed concurrently with the specification. The sequence listing comprised in this ASCII formatted document is part of the specification and is herein incorporated by reference in its entirety.

### FIELD

The disclosure relates to the field of molecular biology, in particular to compositions of novel RNA-guided Cas endonuclease systems, and compositions and methods for editing or modifying the genome of a cell.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs), transcription activator-like effector nucleases (TALENs), or homing meganucleases, are available for producing targeted genome perturbations, but these systems tend to have low specificity and employ designed nucleases that need to be redesigned for each target site, which renders them costly and time-consuming to prepare.

Newer technologies utilizing archaeal or bacterial adaptive immunity systems have been identified, called CRISPR (Clustered Regularly Interspaced Short Palindromic Repeats), which comprise different domains of effector proteins that encompass a variety of activities (DNA recognition, binding, and optionally cleavage).

Despite the identification and characterization of some of these systems, there remains a need for identifying novel effectors and systems, as well as demonstrating activity in eukaryotes, particularly animals and plants, to effect editing of endogenous and previously-introduced heterologous polynucleotides.

Small and robust CRISPR-Cas nucleases are highly desirable for genome editing and related applications. Herein is described a novel Cas endonuclease, “Cas-beta”, exemplary proteins, and methods and compositions for use thereof.

### SUMMARY

Compositions and methods are provided for novel Cas polynucleotides and cas polypeptides as well as other elements derived from a novel CRISPR-Cas locus, including, but not limiting to, additional proteins, CRISPR repeat sequences, and CRISPR arrays comprising CRISPR repeat sequences.

In one aspect, a synthetic composition is provided comprising: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide.

In one aspect, a synthetic composition is provided comprising a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain; and a heterologous polynucleotide.

In one aspect, a synthetic composition is provided comprising a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain; and a heterologous polynucleotide.

In one aspect, a synthetic composition is provided, comprising a CRISPR-Cas effector protein derived from an organism of phylum Armatimonadetes, and a heterologous polynucleotide.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide, wherein said heterologous polynucleotide is derived from an organism of different taxonomy than that from which the effector protein was derived, or is completely or partially artificial.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide, wherein said heterologous polynucleotide is an engineered guide RNA that is functional in a eukaryotic cell.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein further comprises a nuclear localization signal.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is Cas-beta, or a functional fragment thereof.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is a fusion protein comprising a functional fragment of Cas-beta.

In one aspect, a synthetic composition is provided comprising one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is a fusion protein comprising a functional fragment of Cas-beta, wherein said fusion protein further comprises an additional nuclease domain.

In one aspect, a synthetic composition is provided comprising a fusion protein of Cas-beta, or a functional fragment or functional variant thereof, and at least one additional protein selected from the group consisting of: Cas1, Cas2, and a Cas protein that is not Cas1 or Cas2.

In one aspect, a synthetic composition is provided comprising a fusion protein of Cas-beta, or a functional fragment or functional variant thereof, and at least one additional protein selected from the group consisting of: Cas1, Cas2, and a Cas protein that is not Cas1 or Cas2; and further comprising a heterologous polynucleotide or polypeptide.

In one aspect, a synthetic composition is provided comprising: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least one protein, wherein said protein is selected from the group consisting of: Cas1, Cas2, and a Cas protein that is not Cas1 or Cas2.

In one aspect, a synthetic composition is provided comprising: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least one protein, wherein said protein is a complex comprising Cas1, Cas2, and a Cas protein that is not Cas1 or Cas2, in that order.

In one aspect, any of the synthetic compositions described herein may have at least one component optimized for expression in a eukaryotic cell. In some aspects, said cell is from an organism selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato.

In one aspect, a synthetic composition is provided comprising: at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide.

In one aspect, a synthetic composition is provided comprising a polynucleotide encoding comprising a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain.

In one aspect, a synthetic composition is provided comprising a polynucleotide encoding a CRISPR-Cas effector protein comprising a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain; and a heterologous polynucleotide.

In one aspect, a synthetic composition is provided, comprising a polynucleotide encoding a CRISPR-Cas effector protein derived from Armatimonadetes, and a heterologous polynucleotide.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide, wherein said heterologous polynucleotide is derived from an organism of different taxonomy than that from which the effector protein was derived, or is completely or partially artificial.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide, wherein said heterologous polynucleotide is an engineered guide RNA that is functional in a eukaryotic cell.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein further comprises a nuclear localization signal.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is Cas-beta, or a functional fragment thereof.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is a fusion protein comprising a functional fragment of Cas-beta.

In one aspect, a synthetic composition is provided comprising a polynucleotide encoding one of the following: (1) a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, (2) a CRISPR-Cas effector protein comprising at least one domain upstream of the RuvC-I domain, selected from the group consisting of: a positively-charged bridge-helix-like domain and a DNA binding domain optionally comprising a helix-turn-helix motif, wherein said effector protein does not comprise an HNH domain, (3) a CRISPR-Cas effector protein comprising a set of domains in this order: a positively-charged putative bridge-helix-like domain, a DNA binding domain optionally comprising a helix-turn-helix motif, an RuvC-I domain, a bridge-helix domain, an RuvC-II domain, a zinc-finger domain, and an RuvC-III domain; wherein said effector protein does not comprise an HNH domain, and (4) a CRISPR-Cas effector protein derived from Armatimonadetes; and a heterologous polynucleotide; wherein said CRISPR-Cas effector protein is a fusion protein comprising a functional fragment of Cas-beta, wherein said fusion protein further comprises an additional nuclease domain.

In one aspect, a synthetic composition is provided comprising: a polynucleotide encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, and a heterologous polynucleotide, further comprising at least one protein.

In one aspect, a synthetic composition is provided comprising: a polynucleotide a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, and a heterologous polynucleotide, further comprising a polynucleotide encoding at least one protein, wherein said polynucleotide is a gene selected from the group consisting of: cas1, cas2, and one additional cas gene.

In one aspect, a synthetic composition is provided comprising: a polynucleotide encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, further comprising polynucleotide encoding at least one protein, wherein said polynucleotide is a series of the genes cas1, cas2, and one additional cas gene, in that order.

In one aspect, any of the synthetic compositions described herein may have at least one component optimized for expression in a eukaryotic cell. In some aspects, said cell is from an organism selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato

In one aspect, a synthetic composition is provided comprising: a polynucleotide encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain, and a heterologous polynucleotide.

In one aspect, a synthetic composition is provided comprising: a CRISPR-Cas effector protein sharing at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 250, between 250 and 500, at least 500, between 500 and 600, at least 600, between 600 and 700, at least 700, between 700 and 750, at least 750, between 750 and 800, at least 800, between 800 and 825, or greater than 825 contiguous amino acids of SEW NO: 13, 14, 15, or 57; and a heterologous polynucleotide or heterologous polypeptide.

In one aspect, a synthetic composition is provided comprising: a polynucleotide encoding a CRISPR-Cas effector protein sharing at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 250, between 250 and 500, at least 500, between 500 and 600, at least 600, between 600 and 700, at least 700, between 700 and 750, at least 750, between 750 and 800, at least 800, between 800 and 850, at least 850, between 850 and 900, at least 900, between 900 and 950, at least 950, between 950 and 1000, at least 1000, between 1000 and 1250, at least 1250, between 1250 and 1500, at least 1500, between 1500 and 1750, at least 1750, between 1750 and 2000, at least 2000, or greater than 2000 contiguous nucleotides SEW NO: 10, 11, or 12.

In one aspect, an engineered, non-naturally occurring CRISPR-Cas system is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide.

In one aspect, an engineered, non-naturally occurring CRISPR-Cas system is provided comprising: an effector protein system derived from Armatimonadetes, and a heterologous polynucleotide.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said polynucleotide sequence encoding a CRISPR-Cas effector protein shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, between 450 and 500, at least 500, between 500 and 550, at least 550, between 550 and 600, at least 600, between 600 and 650, at least 650, between 650 and 700, at least 700, between 700 and 750, at least 750, between 750 and 800, at least 800, between 800 and 850, at least 850, between 850 and 900, at least 900, between 900 and 950, between 950 and 100, or greater than 1000 contiguous nucleotides of SEQID NO: 10, 11, or 12.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said CRISPR-Cas effector protein shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, between 450 and 500, at least 500, between 500 and 525, at least 525, between 525 amino acids and 540 amino acids, or at least 540 contiguous amino acids of SEW NO: 13, 14, 15, or 57.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least one gene encoding a protein.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; further comprising a plurality of genes encoding at least two different proteins.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least one gene encoding a protein, wherein said gene encoding a protein is selected from the group consisting of: cas1, cas2, and a cas gene that is not cast or cas2 and is optionally cas4.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising a plurality of genes encoding at least two different proteins, wherein the genes in the plurality are in an order selected from the group consisting of: cas1 adjacent to cas2, cas1 not adjacent to a cas gene that is not cas2, and cas2 adjacent to both a cas1 gene and a gene encoding another Cas protein that is not Cas1 or Cast.

In one aspect, a synthetic composition is provided comprising: a polynucleotide sequence encoding a CRISPR-Cas effector protein comprising at least two zinc-finger-like domains, at least two bridge-helix-like domains, an RuvC-I domain, an RuvC-II domain, and an RuvC-III domain; and a heterologous polynucleotide; further comprising a plurality of genes encoding at least two different proteins, wherein the genes in the plurality are in the following order: cas1, cas2, and a gene that is not cas1 or cas2.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least one gene encoding a protein, wherein said polynucleotide sequence encoding a CRISPR-Cas effector protein occurs upstream of at least one gene encoding a protein.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least two genes encoding different proteins, wherein said polynucleotide sequence encoding a CRISPR-Cas effector protein occurs downstream of the at least two genes encoding two different proteins, selected from the group consisting of: cas1, cas2, and a gene that is not cas1 or cas2.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide; further comprising at least three genes encoding different proteins, wherein said polynucleotide sequence encoding a CRISPR-Cas effector protein occurs downstream of the at least three genes encoding three different proteins, selected from the group consisting of: cas1, cas2, and a gene that is not cas1 or cas2.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said heterologous polynucleotide comprises a regulatory element capable of effecting transcription of said polynucleotide sequence encoding a CRISPR-Cas effector protein.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said polynucleotide sequence encoding a CRISPR-Cas effector protein has been sequence-optimized for improved expression in a eukaryotic cell.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said polynucleotide sequence encoding a CRISPR-Cas effector protein has been sequence-optimized for improved expression in a eukaryotic cell, wherein said eukaryotic cell is a plant cell, an animal cell, a fungal cell, or a protist.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said polynucleotide sequence encoding a CRISPR-Cas effector protein has been sequence-optimized for improved expression in a eukaryotic cell, wherein said eukaryotic cell further comprises a pre-existing heterologous polynucleotide.

In one aspect, a synthetic composition is provided comprising: a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a heterologous polynucleotide, wherein said polynucleotide sequence encoding a CRISPR-Cas effector protein has been sequence-optimized for improved expression in a eukaryotic cell, wherein said eukaryotic cell is a plant cell, wherein said plant cell is from a plant selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato.

In one aspect, any of the synthetic compositions provided herein may be comprised within a recombinant vector.

In one aspect, any of the synthetic compositions provided herein may be affixed to a solid matrix. Said mechanism of affixment may be covalent or non-covalent.

In one aspect, any of the synthetic compositions provided herein may be further associated with a microparticle, in some aspects said microparticle is selected from the group consisting of: gold particle, tungsten particle, and silicon carbide whisker particle.

In one aspect, a synthetic composition is provided comprising: a eukaryotic cell; a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 500 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises not more than 1000 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises fewer than 875 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 800 amino acids but fewer than 900 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 800 amino acids but fewer than 875 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises fewer than 875 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 800 amino acids but fewer than 900 amino acids.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 800 amino acids, wherein said protein is selected from the group consisting of: Cas1, Cas2, and another protein that is not Cas1 or Cas2.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises fewer than 875 amino acids, wherein said protein is selected from the group consisting of: Cas1, Cas2, and another protein that is not Cas1 or Cas2.

In one aspect, a synthetic composition is provided comprising: a eukaryotic cell; a heterologous CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; at least one protein, and a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence, wherein said heterologous CRISPR-Cas effector protein comprises at least 800 amino acids but fewer than 860 amino acids, wherein said protein is selected from the group consisting of: Cas1, Cas2, and another protein that is not Cas1 or Cas2.

In one aspect, a synthetic composition is provided comprising: a eukaryotic cell; a heterologous molecule selected from the group consisting of: a cas-beta gene capable of expression a Cas-beta effector protein or functional variant or functional fragment thereof in said eukaryotic cell, and a Cas-beta effector protein or a functional variant or functional fragment thereof; and a guide polynucleotide comprising a variable targeting domain that is substantially complementary to a target sequence in the genome of said cell; wherein said guide polynucleotide and Cas-beta effector protein are capable of forming a complex that can recognize, bind to, and optionally nick or cleave said target sequence.

In one aspect, a synthetic composition is provided comprising: a eukaryotic cell; a heterologous molecule selected from the group consisting of: a cas-beta gene capable of expression a Cas-beta effector protein or functional variant or functional fragment thereof in said eukaryotic cell, and a Cas-beta effector protein or a functional variant or functional fragment thereof; and a guide polynucleotide comprising a variable targeting domain that is substantially complementary to a target sequence in the genome of said cell; wherein said guide polynucleotide and Cas-beta effector protein are capable of forming a complex that can recognize, bind to, and optionally nick or cleave said target sequence; wherein said cas-beta gene shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least to at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, between 450 and 500, at least 500, between 500 and 550, at least 550, between 550 and 600, at least 600, between 600 and 650, at least 650, between 650 and 700, at least 700, between 700 and 750, at least 750, between 750 and 800, at least 800, between 800 and 850, at least 850, between 850 and 900, at least 900, between 900 and 950, between 950 and 100, or greater than 1000 contiguous nucleotides of SEQID NO: 10, 11, or 12.

In one aspect, a synthetic composition is provided comprising: a eukaryotic cell; a heterologous molecule selected from the group consisting of: a cas-beta gene capable of expression a Cas-beta effector protein or functional variant or functional fragment thereof in said eukaryotic cell, and a Cas-beta effector protein or a functional variant or functional fragment thereof; and a guide polynucleotide comprising a variable targeting domain that is substantially complementary to a target sequence in the genome of said cell; wherein said guide polynucleotide and Cas-beta effector protein are capable of forming a complex that can recognize, bind to, and optionally nick or cleave said target sequence; wherein said Cas-beta effector protein shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, between 450 and 500, at least 500, between 500 and 550, at least 550, between 550 amino acids and 600 amino acids, or at least 600 contiguous amino acids of SEW NO: 13, 14, 15, or 57.

In one aspect, a synthetic composition is provided comprising: a eukaryotic cell; at least two different heterologous molecules selected from the group consisting of: a cas1 gene capable of expression a Cas1 protein in said eukaryotic cell, a cas2 gene capable of expression a Cas2 protein in said eukaryotic cell, a cas gene capable of expression a Cas protein that is not Cas1 or Cas2 in said eukaryotic cell, a cas-beta gene capable of expression a Cas-beta effector protein in said eukaryotic cell, a Cas1 protein, a Cas2 protein, a Cas protein that is not Cas1 or Cas2, and a Cas-beta effector protein; and a guide polynucleotide comprising a variable targeting domain that is substantially complementary to a target sequence in the genome of said cell; wherein said guide polynucleotide and Cas-beta effector protein are capable of forming a complex that can recognize, bind to, and optionally nick or cleave said target sequence.

In one aspect, a synthetic composition is provided comprising: a eukaryotic cell; a plurality of different heterologous molecules selected from the group consisting of: a cas1 gene capable of expression a Cas1 protein in said eukaryotic cell, a cas2 gene capable of expression a Cas2 protein in said eukaryotic cell, a cas gene capable of expression a Cas protein in said eukaryotic cell, a cas-beta gene capable of expression a Cas-beta effector protein in said eukaryotic cell; and a guide polynucleotide comprising a variable targeting domain that is substantially complementary to a target sequence in the genome of said cell; wherein said guide polynucleotide and the Cas-beta effector protein encoded by the cas-beta gene are capable of forming a complex that can recognize, bind to, and optionally nick or cleave said target sequence, wherein said plurality of heterologous molecules are included within a continuous polynucleotide sequence comprising the following genes in an order selected from the group consisting of: cas-beta adjacent to cas1, cas1 adjacent to cas2, cas2 adjacent to both cas1 and another gene that is not cas1 or cas2, cas-beta adjacent to cas1 which is adjacent to cas2, cas-beta adjacent to cast which is adjacent to cas2 which is adjacent to another cas gene.

In one aspect, a synthetic composition is provided comprising: a eukaryotic cell; a plurality of different heterologous molecules selected from the group consisting of: a Casl protein, a Cas2 protein, a Cas protein that is not Cas1 or Cas2, and a Cas-beta effector protein; and a guide polynucleotide comprising a variable targeting domain that is substantially complementary to a target sequence in the genome of said cell; wherein said guide polynucleotide and the Cas-beta effector protein are capable of forming a complex that can recognize, bind to, and optionally nick or cleave said target sequence, wherein said plurality of heterologous molecules are included within a continuous polypeptide sequence comprising the following proteins in an order selected from the group consisting of: Cas-beta adjacent to Cas1, Cas1 adjacent to Cas2, Cas2 adjacent to both Casland another gene that is not Cas1 or Cas2, Cas-beta adjacent to Cas1 which is adjacent to Cas2, Cas-beta adjacent to Cas1 which is adjacent to Cas2 which is adjacent to another cas protein that is not Cas1 or Cas2.

In one aspect, a synthetic composition is provided comprising eukaryotic cell, and a heterologous CRISPR-Cas array comprising at least two repeats at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% identical to SEQID NO: 19, 20, or 21. In some aspects, said repeats are separated by a spacer polynucleotide of at least 20, 21, 22, 23, 24, 25, 26, 27, 28, 29, 30, 31, 32, 33, 34, 35, or greater than 35 nucleotides.

In one aspect, a synthetic composition is provided comprising eukaryotic cell, and a heterologous CRISPR-Cas array comprising at least three repeats at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% identical to SEQID NO: 19, 20, or 21. In some aspects, said repeats are separated by spacer polynucleotides of at least 20, 21, 22, 23, 24, 25, 26, 27, 28, 29, 30, 31, 32, 33, 34, 35, or greater than 35 nucleotides.

In one aspect, a synthetic composition is provided comprising eukaryotic cell, and a heterologous CRISPR-Cas array comprising at least four, five, six, seven, eight, nine, ten, 11, 12, 13, 14, 15, 16, 17, 18, 19, 20, 21, 22, 23, 24, 25, 26, 27, 28, 29, 30, 31, 32, 33, 34,35, 36, or 37 repeats at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% identical to SEQID NO: 19, 20, or 21. In some aspects, said repeats are separated by spacer polynucleotides of at least 20, 21, 22, 23, 24, 25, 26, 27, 28, 29, 30, 31, 32, 33, 34, 35, or greater than 35 nucleotides.

In one aspect, in any of the disclosed compositions or methods, a eukaryotic cell further comprising a pre-existing heterologous polynucleotide is provided.

In one aspect, a method is provided for modifying a target sequence in the genome of a eukaryotic cell, comprising: introducing into said cell a composition selected from the group consisting of: comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide or functional variant or functional fragment thereof; SEQID NO: 10, 11, or 12; and SEQID NO: 13, 14, 15, or 57; further comprising a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide or a functional variant or functional fragment thereof; a polynucleotide sharing at least 70%-100% identity with SEQID NO: 10, 11, or 12; and a polypeptide sharing at least 70%-100% identity with SEQID NO: 13, 14, 15, or 57; wherein said guide polynucleotide and CRISPR-Cas effector protein are capable of forming a complex that can recognize, bind to, and optionally nick or cleave said target sequence.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide or a functional fragment or functional variant thereof; SEQID NO: 3; and SEQID NO: 4; further comprising at least one composition selected from the group consisting of: cas1 polynucleotide, Cas1 polypeptide, cas2 polynucleotide, Cas2 polypeptide, cas polynucleotide that is not cast or cas2, a polynucleotide sequence of the sequence listing, and a polypeptide of the sequence listing, Cas polypeptide that is not Cas1 or Cas2, a heterologous polynucleotide, and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment OR functional variant of one or more of said compositions may be substituted.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide; a polynucleotide sequence of the sequence listing, and a polypeptide of the sequence listing; further comprising at least one composition selected from the group consisting of: cas1 polynucleotide, Cas1 polypeptide, cas2 polynucleotide, Cas2 polypeptide, cas4 polynucleotide, a Cas4 polypeptide, a heterologous polynucleotide, a polynucleotide sharing at least 70%-100% identity with any of the polypeptides of the sequence listing; and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment of one or more of said compositions may be substituted for the whole molecule.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide or a functional fragment or functional variant thereof; SEQID NO: 3; and SEQID NO: 4; further comprising at least two compositions selected from the group consisting of: cas1 polynucleotide, Cas1 polypeptide, cas2 polynucleotide, Cas2 polypeptide, cas polynucleotide that is not cas1 or cas2, Cas polypeptide that is not Cas1 or Cas2, a heterologous polynucleotide, a polynucleotide sequence of the sequence listing, and a polypeptide of the sequence listing; and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment OR functional variant of one or more of said compositions may be substituted.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide; a polynucleotide sharing at least 70%-100% identity with SEQID NO: 10, 11, or 12; and a polypeptide sharing at least 70%-100% identity with SEQID NO: 13, 14, 15, or 57; further comprising at least two compositions selected from the group consisting of: casl polynucleotide, Cas1 polypeptide, cast polynucleotide, Cas2 polypeptide, cas4 polynucleotide, a Cas polypeptide that is not Cas1 or Cas2, a heterologous polynucleotide, a polynucleotide sequence of the sequence listing, and a polypeptide of the sequence listing; and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment of one or more of said compositions may be substituted for the whole molecule.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide or a functional fragment or functional variant thereof; SEQID NO: 3; and SEQID NO: 4; further comprising at least three compositions selected from the group consisting of: cas1 polynucleotide, Cas1 polypeptide, cas2 polynucleotide, Cas2 polypeptide, cas polynucleotide that is not cast or cas2, Cas polypeptide that is not Cas1 or Cas2, a heterologous polynucleotide, a polynucleotide sequence of the sequence listing, and a polypeptide of the sequence listing; and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment OR functional variant of one or more of said compositions may be substituted.

In one aspect, a method is provided for modifying a target sequence in the genome of a eukaryotic cell, comprising: introducing into said cell a composition selected from the group consisting of: a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a polynucleotide sequence encoding a CRISPR-Cas effector protein comprising at least two bridge-helix-like domains, a DNA binding domain optionally comprising a helix-turn-helix motif, an RuvC-I domain, an RuvC-II domain, and an RuvC-III domain, wherein said effector protein does not comprise an HNH domain; a cas-beta polynucleotide; a Cas-beta polypeptide; a polynucleotide sharing at least 70%-100% identity with SEQID NO: 10, 11, or 12; and a polypeptide sharing at least 70%-100% identity with SEQID NO: 13, 14, 15, or 57; further comprising at least three compositions selected from the group consisting of: casl polynucleotide, Cas1 polypeptide, cast polynucleotide, Cas2 polypeptide, cas4 polynucleotide, a Cas polypeptide that is not Cas1 or Cas2, a heterologous polynucleotide, a polynucleotide sequence of the sequence listing, and a polypeptide of the sequence listing; and a guide polynucleotide comprising a variable targeting domain that is substantially complementary to a target sequence in the genome of said cell, and incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence. In some embodiments, one or more of said compositions may be optimized for expression in a particular organism or cell type. In some embodiments, a functional fragment of one or more of said compositions may be substituted for the whole molecule.

In any of the methods or compositions provided herein, it is further provided a method or composition further comprising a eukaryotic cell. In some embodiments, the eukaryotic cell is an animal cell. In some embodiments, the eukaryotic cell is a plant cell. In some embodiments, the plant cell is from a plant selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato. In some embodiments, the eukaryotic cell may further comprise a pre-existing heterologous polynucleotide. In some embodiments, the animal cell may further comprise a pre-existing heterologous polynucleotide. In some embodiments, the plant cell may further comprise a pre-existing heterologous polynucleotide.

In one aspect, a polynucleotide modification template is provided comprising at least one region that is complementary to a PAM sequence adjacent to a DNA target sequence recognized by a Cas-beta complex, wherein the at least one region that corresponds to a PAM sequence comprises at least one nucleotide mismatch.

In one aspect, a method of producing a plant with a modified genome is provided , comprising: introducing into at least one cell of said plant a heterologous composition comprising a Cas-beta effector protein or a cas-beta polynucleotide encoding a Cas-beta effector protein, a guide polynucleotide comprising a variable targeting domain that is substantially complementary to a target sequence in the genome of said cell wherein said guide polynucleotide and Cas-beta endonuclease are capable of forming a complex that can recognize, bind to, and optionally nick or cleave said target sequence, and a polynucleotide modification template comprising at least one region that is complementary to a PAM sequence adjacent to a DNA target sequence recognized by a Cas-beta complex, wherein the at least one region that corresponds to a PAM sequence comprises at least one nucleotide mismatch; incubating said cell at a temperature greater than 10 degrees Celsius for a period of at least about four hours; generating a whole plant from said cell; and verifying at least one nucleotide modification in the genome of at least one cell of said plant as compared to the target sequence of the genome of said cell prior to the introduction of said heterologous composition.

In one aspect, a progeny of a plant with a modified genome is provided produced by the method comprising: introducing into at least one cell of said plant a heterologous composition comprising a Cas-beta effector protein or a cas-beta polynucleotide encoding a Cas-beta effector protein, a guide polynucleotide comprising a variable targeting domain that is substantially complementary to a target sequence in the genome of said cell wherein said guide polynucleotide and Cas-beta endonuclease are capable of forming a complex that can recognize, bind to, and optionally nick or cleave said target sequence, and a polynucleotide modification template comprising at least one region that is complementary to a PAM sequence adjacent to a DNA target sequence recognized by a Cas-beta complex, wherein the at least one region that corresponds to a PAM sequence comprises at least one nucleotide mismatch; incubating said cell at a temperature greater than 10 degrees Celsius for a period of at least about four hours; generating a whole plant from said cell; and verifying at least one nucleotide modification in the genome of at least one cell of said plant as compared to the target sequence of the genome of said cell prior to the introduction of said heterologous composition.

In some aspects, the progeny of plants produced by any of the methods or with any of the compositions disclosed herein, a plant element or a plant reproductive element derived from said plants or progeny, and a plurality of plants, progeny, plant elements, and plant reproductive elements are provided.

In some aspects, the plants provided herein include maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato.

In any aspect, a Cas-beta protein may be identical to SEW NO: 13, 14, 15, or 57, or may be a functional fragment or functional variant thereof, or may share 70-100% identity with said sequences. In any aspect, a cas-beta gene may encode a Cas-beta protein or a functional fragment or functional variant thereof.

Any of the methods or compositions herein may further comprise a heterologous polynucleotide. The heterologous polynucleotide may be selected from the group consisting of: a noncoding regulatory expression element such as a promoter, intron, enhancer, or terminator; a donor polynucleotide; a polynucleotide modification template, optionally comprising at least one nucleotide modification as compared to the sequence of a polynucleotide in a cell; a transgene; a guide RNA; a guide DNA; a guide RNA-DNA hybrid; an endonuclease; a nuclear localization signal; and a cell transit peptide.

In one aspect, methods are provided for using any of the compositions disclosed herein. In some embodiments, methods are provided for a Cas-beta endonuclease to bind to a target sequence of a polynucleotide, for example in the genome of a cell or in vitro. In some embodiments, the Cas-beta endonuclease forms a complex with a guide polynucleotide, for example a guide RNA. In some embodiments, the complex recognizes, binds to, and optionally creates a nick (one strand) or a break (two strands) in the polynucleotide at or near the target sequence. In some embodiments, the nick or break is repaired via Non-Homologous End Joining (NHEJ). In some embodiments, the nick or break is repaired via Homology-Directed Repair (HDR) or via Homologous Recombination (HR), with a polynucleotide modification template or a donor DNA molecule.

The novel Cas endonucleases described herein are capable of creating a double-strand break in, or adjacent to, a target polynucleotide that comprises an appropriate PAM, and to which it is directed by a guide polynucleotide, in any prokaryotic or eukaryotic cell. In some cases, the cell is a plant cell or an animal cell or a fungal cell. In some cases, a plant cell is selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, tobacco, Arabidopsis, safflower, and tomato.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 is a graphical overview of the entire Cas-beta CRISPR locus, comprising, from 5′ to 3′: a cas-beta gene, a cas1 gene, a cast gene, cas4 gene, and a CRISPR array. For Cas-betal, the locus comprises 37 repeats of 37 nucleotides with 3 of them (numbers 5, 19, and 30 from the 5′ end) sharing 97.3% identity to the array repeat sequence (SEQID NO: 19, 20, or 21) and the final repeat sharing 78.4% identity to SEQID NO: 19, 20, or 21. For Cas-beta3, only 1 repeat was found in the array.

FIG. 2 is the domain architecture of a Cas-beta protein, showing a R/K/Q-rich bridge-helix like domain, a helix-turn-helix domain, and a split RuvC domain comprising a bridge helix domain between RuvC-I and RuvC-II.

FIG. 3 shows the phylogenetic relationship between Cas-beta proteins and other Cas endonucleases, using evolutionary analysis by a maximum likelihood method. The evolutionary history was inferred by using the Maximum Likelihood method and JTT matrix-based model. The tree with the highest log likelihood (-79975.80) is shown. Initial tree(s) for the heuristic search were obtained automatically by applying Neighbor-Join and BioNJ algorithms to a matrix of pairwise distances estimated using a JTT model, and then selecting the topology with superior log likelihood value. The tree is drawn to scale, with branch lengths measured in the number of substitutions per site. This analysis involved 30 amino acid sequences. There were a total of 1639 positions in the final dataset. Evolutionary analyses were conducted in MEGA X.

FIG. 4 shows conserved features in Cas-beta tracrRNAs.

FIG. 5 depicts a method for Cas-beta expression and target strand recognition and cleavage detection.

FIG. 6A-6E show cleavage of a target polynucleotide with various Cas-beta compositions. FIG. 6A shows no cleavage for the negative control. FIG. 6B shows target double-strand break cleavage for the Cas-beta2 complete locus at 37 degrees Celsius. FIG. 6C shows target double-strand break for the Cas-beta2 complete locus at 40 degrees Celsius. FIG. 6D shows target double-strand break for the Cas-beta2 complete locus at 45 degrees Celsius. FIG. 6E shows target double-strand break for the Cas-beta2 complete locus at 50 degrees Celsius.

FIG. 7A depicts some sequence motifs and domains of a Cas-beta, as shown on the sequence of Cas-betal (SEQID NO:13). FIG. 7B depicts domains and motifs of Cas-beta (as depicted for Cas-betal). BH =bridge helix rich in positively charged K/R/H, for DNA/RNA recognition; TSL=target-strand loading; Helical=helical bundle domain, DNA/RNA binding element; Wed=DNA/RND binding domain, like a wedge on splitting chain fork point.

FIG. 8A shows no cleavage for the negative control (Cas-beta proteins 1 and 3).

FIG. 8B shows target double-strand break cleavage for the Cas-betal complete locus at 37 degrees Celsius. FIG. 8C shows target double-strand break cleavage for the Cas-beta3 complete locus at 37 degrees Celsius.

FIG. 9A shows in vitro reconstitution of double strand DNA target cleavage activity for Cas-beta2 and sgRNA targeting the T2 site. FIG. 9B shows in vitro reconstitution of double strand DNA target cleavage activity for Cas-beta3 and sgRNA targeting the T2 site. FIG. 9C shows in vitro reconstitution of double strand DNA target cleavage activity for Cas-beta2 and sgRNA targeting the T2 site. FIG. 9D shows that Cas-beta2 target cleavage (supercoiled plasmid DNA) is more efficient with a sgRNA comprised of a 24 nt length variable targeting domain as compared to a sgRNA containing a 20 nt length variable targeting domain. FIG. 9E shows that Cas-beta2 RNP digests confirm that both a C-rich PAM immediately 5′ of a protospacer target and a sgRNA with homology to the target are required for the observed site specific cleavage. SEQID NO. 65 and 61 correspond to the T1 and T2 targeting sgRNAs, respectively.

FIG. 10 depicts features of the Cas-beta target site recognition.

FIG. 11A depicts that Cas-beta2 is triggered to non-specifically degrade single stranded DNA after double or single stranded DNA target recognition. FIG. 11B depicts that Cas-beta3 is triggered to non-specifically degrade single stranded DNA after double or single stranded DNA target recognition.

FIG. 12 illustrates an example of a plant optimized Cas-beta expression construct.

FIG. 13 illustrates an example of a plant optimized gRNA expression construct.

FIG. 14A shows no cleavage for the negative control (Cas-beta4 protein). FIG. 14B shows target double-strand break cleavage using Cas-beta4 ribonucleoprotein (RNP) at 37 degrees Celsius.

FIG. 15 depicts robust cleavage by Cas-beat2 of a single target site in the RUNX1 gene when using linear double stranded DNA as substrate.

FIG. 16 depicts RUNX1 site 1 and WTAP site 1 substrates generated for analyzing Cas-beta cleavage preferences by capillary electrophoresis. The target comprised of PAM and protospacer target is capitalized. The PAM is underlined.

FIGS. 17A and 17B depict both Cas-beta 2 and 3 cleaved RUNX1 site 1 while WTAP site 1 failed to produce appreciable levels of cleavage, under conditions of 37 degrees Celsius.

FIGS. 18A and 18B shows that when incubated at 47° C., Cas-beta3 cleaved both substrates while cleavage of WTAP site 1 by Cas-beta2 remains recalcitrant.

FIG. 19 shows Cas-beta2 robustly cleaved both target sites when presented in a supercoiled state and only cleaved the RUNX1 site 1 in a linear form.

FIG. 20A shows that the Cas-beta2 RNP complex cleaved 5 of the 10 targets (WTAP sites 6 and 7 and RUNX1 sites 3, 4 and 7).

FIG. 20B shows that at 37° C., Cas-beta3 only robustly cleaved WTAP site 7 while at 47° C., it cut 8 of the 10 targets.

FIG. 21 shows that Cas-beta2 preferred a sgRNA with a 20 nt length spacer when using linear double stranded DNA substrate.

FIG. 22 shows that Cas-beta3 was more tolerant of sgRNAs with longer spacer lengths.

FIG. 23 shows that both Cas-beta2 and Cas-beta3 left 5′ overhanging termini that varied in length from 6-8 nts upon supercoiled target cleavage.

FIG. 24 shows that, when nucleofected as NLS-tagged RNP into HEK293 cells, Cas-beta2 produced targeted chromosomal DNA sequence alterations when paired with a sgRNA targeting RUNX1 site 1 (SEQID NO:143). These comprised deletion mutations that coincided with the expected cut-sites of Cas-beta2 and disrupted protospacer target recognition. The absence of similar mutations in control experiments confirmed that the changes were due to the introduction of Cas-beta2 RNP, altogether, providing the first evidence for double stranded DNA target binding and cleavage activity in a eukaryotic cell. Lower case nucleotides represent additional bases inserted as part of double-strand break repair. Dashes represent deleted bases.

FIGS. 25 and 26 depict the recovery of deletion mutations spanning the expected Cas-beta2 cut-sites when Cas-beta2 sgRNAs (SEQID NOs: 144 and 145) capable of base pairing with the complementary strand of WTAP sites 6 and 7 were generated by T7 in vitro transcription, complexed with Cas-beta2 NLS-tagged protein, and delivered into HEK293 cells using electroporation. The deletion resulting from the repair of Cas-beta target recognition and cleavage was large. To view the alignment between mutated reads and reference, some bases were removed from the reference. The position and number of bases removed are indicated between two “I”. The sequence listing contains the full reference sequence. Lower case nucleotides represent additional bases inserted as part of double-strand break repair. Dashes represent deleted bases.

FIG. 27 demonstrates that Cas-beta3 also functions in HEK293 cells to produce a targeted mutation at the WTAP site 7. The deletion resulting from the repair of Cas-beta target recognition and cleavage was large. To view the alignment between mutated read and reference, some bases were removed from the reference. The position and number of bases removed are indicated between two “I”. The sequence listing contains the full reference sequence. Dashes represent deleted bases.

FIG. 28 shows that Cas-beta nuclease and sgRNA can function in other eukaryotic cells. Here, when expression cassettes encoding Cas-beta2 and sgRNA were delivered into immature embryos (IEs) using biolistics, targeted mutations were recovered at both male sterile 26 (MS26) gene targets two days after transformation. Dashes represent deleted bases.

FIG. 29 shows that when the recipient strain, E. coli Arctic Express (DE3), carrying Cas-beta2 nuclease and guide RNA expression cassettes was transformed with the target plasmid, no colonies were obtained, altogether, demonstrating Cas-beta nuclease and sgRNA functionality to a heterologous prokaryotic cell. Dashes represent deleted bases.

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

SEQID NO:1 is the Armatimonadetes bacterium PRT sequence for the Cas1 encoded in Cas-beta 1 locus.

SEQID NO:2 is the Armatimonadetes bacterium PRT sequence for the Cas1 encoded in Cas-beta 2 locus.

SEQID NO:3 is the Armatimonadetes bacterium PRT sequence for the Cas1 encoded in Cas-beta 3 locus.

SEQID NO:4 is the Armatimonadetes bacterium PRT sequence for the Cas2 encoded in Cas-beta 1 locus.

SEQID NO:5 is the Armatimonadetes bacterium PRT sequence for the Cas2 encoded in Cas-beta 2 locus.

SEQID NO:6 is the Armatimonadetes bacterium PRT sequence for the Cas2 encoded in Cas-beta 3 locus.

SEQID NO:7 is the Armatimonadetes bacterium PRT sequence for the Cas4 encoded in Cas-beta 1 locus.

SEQID NO:8 is the Armatimonadetes bacterium PRT sequence for the Cas4 encoded in Cas-beta 2 locus.

SEQID NO:9 is the Armatimonadetes bacterium PRT sequence for the Cas4 encoded in Cas-beta 3 locus.

SEQID NO:10 is the Armatimonadetes bacterium DNA sequence for the Cas-beta 1 endonuclease gene.

SEQID NO:11 is the Armatimonadetes bacterium DNA sequence for the Cas-beta 2 endonuclease gene.

SEQID NO:12 is the Armatimonadetes bacterium DNA sequence for the Cas-beta 3 endonuclease gene.

SEQID NO:13 is the Armatimonadetes bacterium PRT sequence for the Cas-beta 1 endonuclease.

SEQID NO:14 is the Armatimonadetes bacterium PRT sequence for the Cas-beta 2 endonuclease.

SEQID NO:15 is the Armatimonadetes bacterium PRT sequence for the Cas-beta 3 endonuclease.

SEQID NO:16 is the Armatimonadetes bacterium DNA sequence for the Cas-beta 1 locus.

SEQID NO:17 is the Armatimonadetes bacterium DNA sequence for the Cas-beta 2 locus.

SEQID NO:18 is the Armatimonadetes bacterium DNA sequence for the Cas-beta 3 locus.

SEQID NO:19 is the Armatimonadetes bacterium DNA sequence for the Cas-beta 1 CRISPR repeat.

SEQID NO:20 is the Armatimonadetes bacterium DNA sequence for the Cas-beta 2 CRISPR repeat.

SEQID NO:21 is the Armatimonadetes bacterium DNA sequence for the Cas-beta 3 CRISPR repeat.

SEQID NO:22 is the Armatimonadetes bacterium RNA sequence for the Cas-beta 1 CRISPR RNA.

SEQID NO:23 is the Armatimonadetes bacterium RNA sequence for the Cas-beta 2 CRISPR RNA.

SEQID NO:24 is the Armatimonadetes bacterium RNA sequence for the Cas-beta 3 CRISPR RNA.

SEQID NO:25 is the Armatimonadetes bacterium RNA sequence for the Cas-beta 1 tracrRNA with terminator-like hairpin.

SEQID NO:26 is the Armatimonadetes bacterium RNA sequence for the Cas-beta 1 tracrRNA without terminator-like hairpin.

SEQID NO:27 is the Armatimonadetes bacterium RNA sequence for the Cas-beta 2 tracrRNA with terminator-like hairpin.

SEQID NO:28 is the Armatimonadetes bacterium RNA sequence for the Cas-beta 2 tracrRNA without terminator-like hairpin.

SEQID NO:29 is the Armatimonadetes bacterium RNA sequence for the Cas-beta 3 tracrRNA with terminator-like hairpin.

SEQID NO:30 is the Armatimonadetes bacterium RNA sequence for the Cas-beta 3 tracrRNA without terminator-like hairpin.

SEQID NO:31 is the Artificial RNA sequence for the Cas-beta 1 sgRNA with terminator-like hairpin.

SEQID NO:32 is the Artificial RNA sequence for the Cas-beta 1 sgRNA without terminator-like hairpin.

SEQID NO:33 is the Artificial RNA sequence for the Cas-beta 2 sgRNA with terminator-like hairpin.

SEQID NO:34 is the Artificial RNA sequence for the Cas-beta 2 sgRNA without terminator-like hairpin.

SEQID NO:35 is the Artificial RNA sequence for the Cas-beta 3 sgRNA with terminator-like hairpin.

SEQID NO:36 is the Artificial RNA sequence for the Cas-beta 3 sgRNA without terminator-like hairpin.

SEQID NO:37 is the Artificial DNA sequence for the T2 PAM library target sequence.

SEQID NO:38 is the Artificial PRT sequence for the 6X histidine tag.

SEQID NO:39 is the Artificial PRT sequence for the 10X histidine tag.

SEQID NO:40 is the Artificial PRT sequence for the Maltose binding protein tag.

SEQID NO:41 is the Tobacco etch virus PRT sequence for the Tobacco etch virus cleavage site.

SEQID NO:42 is the Artificial DNA sequence for the Al oligonucleotide.

SEQID NO:43 is the Artificial DNA sequence for the A2 oligonucleotide.

SEQID NO:44 is the Artificial DNA sequence for the RO oligonucleotide.

SEQID NO:45 is the Artificial DNA sequence for the CO oligonucleotide.

SEQID NO:46 is the Artificial DNA sequence for the F1 oligonucleotide.

SEQID NO:47 is the Artificial DNA sequence for the R1 oligonucleotide.

SEQID NO:48 is the Artificial DNA sequence for the Bridge amplification portion of Fl oligonucleotide.

SEQID NO:49 is the Artificial DNA sequence for the Bridge amplification portion of R1 oligonucleotide.

SEQID NO:50 is the Artificial DNA sequence for the F2 oligonucleotide.

SEQID NO:51 is the Artificial DNA sequence for the R2 oligonucleotide.

SEQID NO:52 is the Artificial DNA sequence for the Cl oligonucleotide.

SEQID NO:53 is the Artificial DNA sequence for the Sequence resulting from cleavage and adapter ligation at position 23 of the target.

SEQID NO:54 is the Artificial DNA sequence for the Adapter portion of SEQID NO. 53.

SEQID NO:55 is the Artificial DNA sequence for the Target portion of SEQID NO. 53.

SEQID NO:56 is the Artificial DNA sequence for the Sequence 5′ of PAM.

SEQID NO:57 is the protein sequence for Cas-beta4 from Archaea.

SEQID NO:58 is the protein sequence for the 14x histidine tag.

SEQID NO:59 is the RNA sequence for the 24 nt spacer sequence (targeting T2).

SEQID NO:60 is the RNA sequence for the Cas-beta 2 sgRNA with terminator-like hairpin targeting T2 sequence.

SEQID NO:61 is the RNA sequence for the Cas-beta 2 sgRNA without terminator-like hairpin targeting T2 sequence.

SEQID NO:62 is the RNA sequence for the Cas-beta 3 sgRNA with terminator-like hairpin targeting T2 sequence.

SEQID NO:63 is the RNA sequence for the Cas-beta 3 sgRNA without terminator-like hairpin targeting T2 sequence.

SEQID NO:64 is the DNA sequence for the T1 target sequence.

SEQID NO:65 is the RNA sequence for the Cas-beta 2 sgRNA without terminator-like hairpin targeting Ti sequence.

SEQID NO:66 is the RNA sequence for the Cas-beta 2 sgRNA without terminator-like hairpin targeting 20 nt of T2 sequence.

SEQID NO:67 is the DNA sequence for the Oligonucleotide activator.

SEQID NO:68 is the DNA sequence for the Oligonucleotide complementary to activator.

SEQID NO:69 is the DNA sequence for the Oligonucleotide 1 used to generate dsDNA non-specific activator.

SEQID NO:70 is the DNA sequence for the Oligonucleotide 2 used to generated dsDNA non-specific activator.

SEQID NO:71 is the Simian virus 40 PRT sequence for the SV40 NLS.

SEQID NO:72 is the Zea mays DNA sequence for the Ubiquitin promoter.

SEQID NO:73 is the Zea mays DNA sequence for the Ubiquitin 5′ UTR.

SEQID NO:74 is the Zea mays DNA sequence for the Ubiquitin intron 1.

SEQID NO:75 is the Artificial DNA sequence for the Sequence encoding SV40 NLS.

SEQID NO:76 is the Artificial DNA sequence for the Exon 1 of maize optimized Cas-beta 1 nuclease.

SEQID NO:77 is the Artificial DNA sequence for the Exon 2 of maize optimized Cas-beta 1 nuclease.

SEQID NO:78 is the Solanum tuberosum DNA sequence for the ST-LS1 Intron 2.

SEQID NO:79 is the Solanum tuberosum DNA sequence for the Potato Proteinase Inhibitor II terminator.

SEQID NO:80 is the Zea mays DNA sequence for the U6 promoter with a 3′ G bp.

SEQID NO:81 is the Artificial DNA sequence for the Sequence encoding Cas-beta Affinity Domain of sgRNA.

SEQID NO:82 is the Zea mays DNA sequence for the Sequence encoding Variable Targeting Domain of sgRNA targeting Zea mays male sterile 26 gene.

SEQID NO:83 is the Hepatitis delta virus DNA sequence for the HDV ribozyme sequence.

SEQID NO:84 is the Zea mays DNA sequence for the U6 terminator.

SEQID NO:85 is the Armatimonadetes bacterium PRT sequence for the Cas2 encoded in Cas-beta 4 locus.

SEQID NO:86 is the Armatimonadetes bacterium PRT sequence for the Cas4 encoded in Cas-beta 4 locus.

SEQID NO:87 is the Armatimonadetes bacterium DNA sequence for the Cas-beta 4 endonuclease gene.

SEQID NO:88 is the Armatimonadetes bacterium DNA sequence for the Cas-beta 4 locus.

SEQID NO:89 is the Armatimonadetes bacterium RNA sequence for the Cas-beta 4 tracrRNA with terminator-like hairpin.

SEQID NO:90 is the Armatimonadetes bacterium RNA sequence for the Cas-beta 4 tracrRNA without terminator-like hairpin.

SEQID NO:91 is the Artificial RNA sequence for the Cas-beta 4 sgRNA with terminator-like hairpin.

SEQID NO:92 is the Artificial RNA sequence for the Cas-beta 4 sgRNA without terminator-like hairpin.

SEQID NO:93 is the Homo sapiens DNA sequence for the WTAP site 1.

SEQID NO:94 is the Homo sapiens DNA sequence for the WTAP site 2.

SEQID NO:95 is the Homo sapiens DNA sequence for the RUNX1 site 1.

SEQID NO:96 is the Homo sapiens DNA sequence for the RUNX1 site 2.

SEQID NO:97 is the Artificial RNA sequence for the Cas-beta2 sgRNA targeting WTAP site 1.

SEQID NO:98 is the Artificial RNA sequence for the Cas-beta2 sgRNA targeting WTAP site 2.

SEQID NO:99 is the Artificial RNA sequence for the Cas-beta2 sgRNA targeting RUNX1 site 1.

SEQID NO:100 is the Artificial RNA sequence for the Cas-beta2 sgRNA targeting RUNX1 site 2.

SEQID NO:101 is the Artificial RNA sequence for the Cas-beta3 sgRNA targeting WTAP site 1.

SEQID NO:102 is the Artificial RNA sequence for the Cas-beta3 sgRNA targeting WTAP site 2.

SEQID NO:103 is the Artificial RNA sequence for the Cas-beta3 sgRNA targeting RUNX1 site 1.

SEQID NO:104 is the Artificial RNA sequence for the Cas-beta3 sgRNA targeting RUNX1 site 2.

SEQID NO:105 is the Artificial DNA sequence for the 5′ FAM labeled RUNX1 site 1 non-target strand.

SEQID NO:106 is the Artificial DNA sequence for the 5′ ROX labeled RUNX1 site 1 target strand.

SEQID NO:107 is the Artificial DNA sequence for the 5′ FAM labeled WTAP site 1 non-target strand.

SEQID NO:108 is the Artificial DNA sequence for the 5′ ROX labeled WTAP site 1 target strand.

SEQID NO:109 is the Homo sapiens DNA sequence for the WTAP site 3.

SEQID NO:110 is the Homo sapiens DNA sequence for the WTAP site 4.

SEQID NO:111 is the Homo sapiens DNA sequence for the WTAP site 5.

SEQID NO:112 is the Homo sapiens DNA sequence for the WTAP site 6.

SEQID NO:113 is the Homo sapiens DNA sequence for the WTAP site 7.

SEQID NO:114 is the Homo sapiens DNA sequence for the RUNX1 site 3.

SEQID NO:115 is the Homo sapiens DNA sequence for the RUNX1 site 4.

SEQID NO:116 is the Homo sapiens DNA sequence for the RUNX1 site 5.

SEQID NO:117 is the Homo sapiens DNA sequence for the RUNX1 site 6.

SEQID NO:118 is the Homo sapiens DNA sequence for the RUNX1 site 7.

SEQID NO:119 is the Artificial DNA sequence for the Non-target strand (NTS).

SEQID NO:120 is the Artificial DNA sequence for the Sanger sequence from the reverse direction (Cas-beta2).

SEQID NO:121 is the Artificial DNA sequence for the Target strand (TS).

SEQID NO:122 is the Artificial DNA sequence for the Sanger sequence from the forward direction (Cas-beta2).

SEQID NO:123 is the Artificial DNA sequence for the Sanger sequence from the reverse direction (Cas-beta3).

SEQID NO:124 is the Artificial DNA sequence for the Sanger sequence from the forward direction (Cas-beta3).

SEQID NO:125 is the Homo sapiens DNA sequence for the RUNX1 site 1 reference.

SEQID NO:126 is the Homo sapiens DNA sequence for the Cas-beta2 RUNX1 site 1 mutation 1.

SEQID NO:127 is the Homo sapiens DNA sequence for the Cas-beta2 RUNX1 site 1 mutation 2.

SEQID NO:128 is the Homo sapiens DNA sequence for the Cas-beta2 RUNX1 site 1 mutation 3.

SEQID NO:129 is the Homo sapiens DNA sequence for the Cas-beta2 RUNX1 site 1 mutation 4.

SEQID NO:130 is the Homo sapiens DNA sequence for the Cas-beta2 RUNX1 site 1 mutation 5.

SEQID NO:131 is the Homo sapiens DNA sequence for the Cas-beta2 RUNX1 site 1 mutation 6.

SEQID NO:132 is the Homo sapiens DNA sequence for the WTAP site 6 reference.

SEQID NO:133 is the Homo sapiens DNA sequence for the Cas-beta2 WTAP site 6 mutation 1.

SEQID NO:134 is the Homo sapiens DNA sequence for the Cas-beta2 WTAP site 6 mutation 2.

SEQID NO:135 is the Homo sapiens DNA sequence for the Cas-beta2 WTAP site 6 mutation 3.

SEQID NO:136 is the Homo sapiens DNA sequence for the Cas-beta2 WTAP site 6 mutation 4.

SEQID NO:137 is the Homo sapiens DNA sequence for the Cas-beta2 WTAP site 6 mutation 5.

SEQID NO:138 is the Homo sapiens DNA sequence for the WTAP site 7 reference.

SEQID NO:139 is the Homo sapiens DNA sequence for the Cas-beta2 or 3 WTAP site 7 mutation 1.

SEQID NO:140 is the Homo sapiens DNA sequence for the Cas-beta2 WTAP site 7 mutation 2.

SEQID NO:141 is the Homo sapiens DNA sequence for the Cas-beta2 WTAP site 7 mutation 3.

SEQID NO:142 is the Homo sapiens DNA sequence for the Cas-beta2 WTAP site 7 mutation 4.

SEQID NO:143 is the Artificial RNA sequence for the Cas-beta2 sgRNA targeted RUNX1 site 1 (20 nt spacer).

SEQID NO:144 is the Artificial RNA sequence for the Cas-beta2 sgRNA targeted WTAP site 6 (20 nt spacer).

SEQID NO:145 is the Artificial RNA sequence for the Cas-beta2 sgRNA targeted WTAP site 7 (20 nt spacer).

SEQID NO:146 is the Artificial RNA sequence for the Cas-beta3 sgRNA targeted WTAP site 7 (20 nt spacer).

SEQID NO:147 is the Zea mays DNA sequence for the Reference MS26 site 1.

SEQID NO:148 is the Zea mays DNA sequence for the MS26 site 1 Mutation.

SEQID NO:149 is the Zea mays DNA sequence for the Reference MS26 site 2.

SEQID NO:150 is the Zea mays DNA sequence for the MS26 site 2 Mutation.

SEQID NO:151 is the Artificial DNA sequence for the Cas-beta2 native spacer 1 target.

SEQID NO:152 is the Artificial DNA sequence for the Cas-beta2 native spacer 2 target.

SEQID NO:153 is the Artificial DNA sequence for the Cas-beta2 native spacer 3 target.

SEQID NO:154 is the Artificial PRT sequence for the D528A cleavage inactive Cas-beta2.

SEQID NO:155 is the Artificial PRT sequence for the E634A cleavage inactive Cas-beta2.

### DETAILED DESCRIPTION

Compositions and methods are provided for novel CRISPR effector systems and elements comprising such systems, including, but not limiting to, novel guide polynucleotide/endonuclease complexes, guide polynucleotides, guide RNA elements, Cas proteins, and endonucleases, as well as proteins comprising an endonuclease functionality (domain). Compositions and methods are also provided for direct delivery of endonucleases, cleavage ready complexes, guide RNAs, and guide RNA/Cas endonuclease complexes. The present disclosure further includes compositions and methods for genome modification of a target sequence in the genome of a cell, for gene editing, and for inserting a polynucleotide of interest into the genome of a cell.

The small, novel Cas endonucleases described herein, “Cas-beta”, are derived from a CRISPR locus with a unique architecture, comprising in this order: a cas-beta gene, a cas1 gene, a cast gene, a gene with low identity to cas4, and a CRISPR array. The cas-beta gene encodes a Cas-beta endonuclease that is less than 900 amino acids and has a unique protein architecture.

Terms used in the claims and specification are defined as set forth below unless otherwise specified. It must be noted that, as used in the specification and the appended claims, the singular forms “a,” “an” and “the” include plural referents unless the context clearly dictates otherwise.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. Nucleotides (usually found in their 5′-monophosphate form) are referred to by their single letter designation as follows:“A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1X to 2X SSC (20X SSC =3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5X to 1X SSC at 55 to 60° C. Exemplary high stringency conditions include hybridization in 50% formamide, 1 M NaCl, 1% SDS at 37° C., and a wash in 0.1X SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events:the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, Calif.) using the following parameters:% identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5X SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in”, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used). examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol 92:1-11 for a discussion of host-preferred codon usage.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed).

“Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-beta protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas-beta endonucleases of the disclosure include those having one or more RuvC nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double-strand break in) the target site is retained. The portion or subsequence of the Cas endonuclease can comprise a complete or partial (functional) peptide of any one of its domains such as for example, but not limiting to a complete of functional part of a Cas3 HD domain, a complete of functional part of a Cas3 Helicase domain, complete of functional part of a protein (such as but not limiting to a Cas5, Cas5d, Cas? and Cas8b1).

The terms “functional variant”, “variant that is functionally equivalent” and “functionally equivalent variant” of a Cas endonuclease or Cas effector protein, including Cas-beta described herein, are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 February 2015), or any combination thereof.

As used herein, the terms “guide polynucleotide/Cas endonuclease complex”, “guide polynucleotide/Cas endonuclease system”, “ guide polynucleotide/Cas complex”, “guide polynucleotide/Cas system” and “guided Cas system” “Polynucleotide-guided endonuclease” , “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al, 2015, Cell 163, 1-13; Shmakov et al., 2015, Molecular Cell 60, 1-13).

The terms “guide RNA/Cas endonuclease complex”, “guide RNA/Cas endonuclease system”, “ guide RNA/Cas complex”, “guide RNA/Cas system”, “gRNA/Cas complex”, “gRNA/Cas system”, “RNA-guided endonuclease” , “RGEN” are used interchangeably herein and refer to at least one RNA component and at least one Cas endonuclease that are capable of forming a complex , wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence”, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The term “plant-optimized Cas endonuclease” herein refers to a Cas protein, including a multifunctional Cas protein, encoded by a nucleotide sequence that has been optimized for expression in a plant cell or plant.

A “plant-optimized nucleotide sequence encoding a Cas endonuclease”, “plant-optimized construct encoding a Cas endonuclease” and a “plant-optimized polynucleotide encoding a Cas endonuclease” are used interchangeably herein and refer to a nucleotide sequence encoding a Cas protein, or a variant or functional fragment thereof, that has been optimized for expression in a plant cell or plant. A plant comprising a plant-optimized Cas endonuclease includes a plant comprising the nucleotide sequence encoding for the Cas sequence and/or a plant comprising the Cas endonuclease protein. In one aspect, the plant-optimized Cas endonuclease nucleotide sequence is a maize-optimized, rice-optimized, wheat-optimized, soybean-optimized, cotton-optimized, or canola-optimized Cas endonuclease.

The term “plant” generically includes whole plants, plant organs, plant tissues, seeds, plant cells, seeds and progeny of the same. The plant is a monocot or dicot. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). It should be noted that a protoplast is not technically an “intact” plant cell (as naturally found with all components), as protoplasts lack a cell wall. The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to:seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of a plant.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male- fertile, respectively. It is further recognized that a male fertile (but female sterile) plant can produce viable progeny when crossed with a female fertile plant and that a female fertile (but male sterile) plant can produce viable progeny when crossed with a male fertile plant.

The term “non-conventional yeast” herein refers to any yeast that is not a Saccharomyces (e.g., S. cerevisiae) or Schizosaccharomyces yeast species. (see “Non-Conventional Yeasts in Genetics, Biochemistry and Biotechnology:Practical Protocols”, K. Wolf, K.D. Breunig, G. Barth, Eds., Springer-Verlag, Berlin, Germany, 2003).

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

“Introducing” is intended to mean presenting to a target, such as a cell or organism, a polynucleotide or polypeptide or polynucleotide-protein complex, in such a manner that the component(s) gains access to the interior of a cell of the organism or to the cell itself.

A “polynucleotide of interest” includes any nucleotide sequence encoding a protein or polypeptide that improves desirability of crops, i.e. a trait of agronomic interest. Polynucleotides of interest include, but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### Classification of CRISPR-Cas Systems

CRISPR-Cas systems have been classified according to sequence and structural analysis of components. Multiple CRISPR/Cas systems have been described including Class 1 systems, with multisubunit effector complexes (comprising type I, type III, and type IV), and Class 2 systems, with single protein effectors (comprising type II, type V, and type VI) (Makarova et al. 2015, Nature Reviews Microbiology Vol. 13:1-15; Zetsche et al., 2015, Cell 163, 1-13; Shmakov et al., 2015, Molecular Cell 60, 1-13; Haft et al., 2005, Computational Biology, PLoS Comput Biol 1(6):e60; and Koonin et al. 2017, Curr Opinion Microbiology 37:67-78).

A CRISPR-Cas system comprises, at a minimum, a CRISPR RNA (crRNA) molecule and at least one CRISPR-associated (Cas) protein to form crRNA ribonucleoprotein (crRNP) effector complexes. CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner (Jore et al., Nature Structural & Molecular Biology 18,529-536 (2011)). The crRNA serves as a guide RNA for sequence specific binding of the effector (protein or complex) to double strand DNA sequences, by forming base pairs with the complementary DNA strand while displacing the noncomplementary strand to form a so called R-loop. (Jore et al., 2011. Nature Structural & Molecular Biology 18, 529-536).

RNA transcripts of CRISPR loci (pre-crRNA) are cleaved specifically in the repeat sequences by CRISPR associated (Cas) endoribonucleases in type I and type III systems or by RNase III in type II systems. The number of CRISPR-associated genes at a given CRISPR locus can vary between species.

Different cas genes that encode proteins with different domains are present in different CRISPR systems. The cas operon comprises genes that encode for one or more effector endonucleases, as well as other Cas proteins. Protein subunits include those described in Makarova et al. 2011, Nat Rev Microbiol. 2011 9(6):467-477; Makarova et al. 2015, Nature Reviews Microbiology Vol. 13:1-15; and Koonin et al. 2017, Current Opinion Microbiology 37:67-78). The types of domains include those involved in Expression (pre-crRNA processing, for example Cas 6 or RNaseIII), Interference (including an effector module for crRNA and target binding, as well as domain(s) for target cleavage), Adaptation (spacer insertion, for example Cas1 or Cas2), and Ancillary (regulation or helper or unknown function). Some domains may serve more than one purpose, for example Cas9 comprises domains for endonuclease functionality as well as for target cleavage, among others.

The Cas endonuclease is guided by a single CRISPR RNA (crRNA) through direct RNA-DNA base-pairing to recognize a DNA target site that is in close vicinity to a protospacer adjacent motif (PAM) (Jore, M. M. et al., 2011, Nat. Struct. Mol. Biol. 18:529-536, Westra, E.R. et al., 2012, Molecular Cell 46:595-605, and Sinkunas, T. et al., 2013, EMBO J. 32:385-394).

### Class I CRISPR-Cas Systems

Class I CRISPR-Cas systems comprise Types I, III, and IV. A characteristic feature of Class I systems is the presence of an effector endonuclease complex instead of a single protein. A Cascade complex comprises a RNA recognition motif (RRM) and a nucleic acid-binding domain that is the core fold of the diverse RAMP (Repeat-Associated Mysterious Proteins) protein superfamily (Makarova et al. 2013, Biochem Soc Trans 41, 1392-1400; Makarova et al. 2015, Nature Reviews Microbiology Vol. 13:1-15). RAMP protein subunits include Cas5 and Cas7 (which comprise the skeleton of the crRNA—effector complex), wherein the Cas5 subunit binds the 5′ handle of the crRNA and interacts with the large subunit, and often includes Cas6 which is loosely associated with the effector complex and typically functions as the repeat-specific RNase in the pre-crRNA processing (Charpentier et al., FEMS Microbiol Rev 2015, 39:428-441; Niewoehner et al., RNA 2016, 22:318-329).

Type I CRISPR-Cas systems comprise a complex of effector proteins, termed Cascade (CRISPR-associated complex for antiviral defense) comprising at a minimum Cas5 and Cas7. The effector complex functions together with a single CRISPR RNA (crRNA) and Cas3 to defend against invading viral DNA (Brouns, S.J.J. et al. Science 321:960-964; Makarova et al. 2015, Nature Reviews Microbiology Vol. 13:1-15). Type I CRISPR-Cas loci comprise the signature gene cas3 (or a variant cas3′ or cas3″), which encodes a metal-dependent nuclease that possesses a single-stranded DNA (ssDNA)-stimulated superfamily 2 helicase with a demonstrated capacity to unwind double stranded DNA (dsDNA) and RNA—DNA duplexes (Makarova et al. 2015, Nature Reviews; Microbiology Vol. 13:1-15). Following target recognition, the Cas3 endonuclease is recruited to the Cascade-crRNA-target DNA complex to cleave and degrade the DNA target (Westra, E.R. et al. (2012) Molecular Cell 46:595-605, Sinkunas, T. et al. (2011) EMBO J. 30:1335-1342, and Sinkunas, T. et al. (2013) EMBO J. 32:385-394). In some type I systems, Cas6 can be the active endonuclease that is responsible for crRNA processing, and Cas5 and Cas7 function as non-catalytic RNA-binding proteins; although in type I-C systems, crRNA processing can be catalyzed by Cas5 (Makarova et al. 2015, Nature Reviews Microbiology Vol. 13:1-15). Type I systems are divided into seven subtypes (Makarova et al. 2011, Nat Rev Microbiol. 2011 9(6):467-477; Koonin et al. 2017, Curr Opinion Microbiology 37:67-78). A modified type I CRISPR-associated complex for adaptive antiviral defense (Cascade) comprising at least the protein subunits Cas7, Cas5 and Cas6, wherein one of these subunits is synthetically fused to a Cas3 endonuclease or a modified restriction endonuclease, Fokl, have been described (WO2013098244 published 4 Jul. 4 2013).

Type III CRISPR-Cas systems, comprising a plurality of cas7 genes, target either ssRNA or ssDNA, and function as either an RNase as well as a target RNA-activated DNA nuclease (Tamulaitis et al., Trends in Microbiology 25(10)49-61, 2017). Csm (Type III-A) and Cmr (Type III-B) complexes function as RNA-activated single-stranded (ss) DNases that couple the target RNA binding/cleavage with ssDNA degradation. Upon foreign DNA infection, the CRISPR RNA (crRNA)-guided binding of the Csm or Cmr complex to the emerging transcript recruits Cas10 DNase to the actively transcribed phage DNA, resulting in degradation of both the transcript and phage DNA, but not the host DNA. The Cas10 HD-domain is responsible for the ssDNase activity, and Csm3/Cmr4 subunits are responsible for the endoribonuclease activity of the Csm/Cmr complex. The 3′-flanking sequence of the target RNA is critical for the ssDNase activity of Csm/Cmr: the basepairing with the 5′-handle of crRNA protects host DNA from degradation.

Type IV systems, although comprising typical type I cas5 and cas7 domains in addition to a cas8-like domain, may lack the CRISPR array that is characteristic of most other CRISPR-Cas systems.

### Class II CRISPR-Cas Systems

Class II CRISPR-Cas systems comprise Types II, V, and VI. A characteristic feature of Class II systems is the presence of a single Cas effector protein instead of an effector complex. Types II and V Cas proteins comprise an RuvC endonuclease domain that adopts the RNase H fold.

Type II CRISPR/Cas systems employ a crRNA and tracrRNA (trans-activating CRISPR RNA) to guide the Cas endonuclease to its DNA target. The crRNA comprises a spacer region complementary to one strand of the double strand DNA target and a region that base pairs with the tracrRNA (trans-activating CRISPR RNA) forming a RNA duplex that directs the Cas endonuclease to cleave the DNA target, leaving a blunt end. Spacers are acquired through a not fully understood process involving Cas1 and Cas2 proteins. Type II CRISPR/Cas loci typically comprise cas1 and cast genes in addition to the cas9 gene (Chylinski et al., 2013, RNA Biology 10:726-737; Makarova et al. 2015, Nature Reviews Microbiology Vol. 13:1-15). Type II CRISR-Cas loci can encode a tracrRNA, which is partially complementary to the repeats within the respective CRISPR array, and can comprise other proteins such as Csnl and Csn2. The presence of cas9 in the vicinity of cas1 and cas2 genes is the hallmark of type II loci (Makarova et al. 2015, Nature Reviews Microbiology Vol. 13:1-15).

Type V CRISPR/Cas systems comprise a single Cas endonuclease, including Cpf1 (Cas12) (Koonin et al., Curr Opinion Microbiology 37:67-78, 2017), that is an active RNA-guided endonuclease that does not necessarily require the additional trans-activating CRISPR (tracr) RNA for target cleavage, unlike Cas9.

Type VI CRISPR-Cas systems comprise a cas13 gene that encodes a nuclease with two HEPN (Higher Eukaryotes and Prokaryotes Nucleotide-binding) domains but no HNH or RuvC domains, and are not dependent upon tracrRNA activity. The majority of HEPN domains comprise conserved motifs that constitute a metal-independent endoRNase active site (Anantharam et al., Biol Direct 8:15, 2013). Because of this feature, it is thought that type VI systems act on RNA targets instead of the DNA targets that are common to other CRISPR-Cas systems.

### Novel CRISPR-Cas Systems

Disclosed herein is a novel CRISPR-Cas system, components thereof, and methods of using said components. The system comprises a novel Cas effector protein, Cas-beta.

The novel CRISPR-Cas system components described herein may comprise one or more subunits from different Cas systems, subunits derived or modified from more than one different bacterial or archaeal prokaryote, and/or synthetic or engineered components.

Described herein is a newly identified CRISPR-Cas system comprising novel arrangements of cas genes. Further described are novel cas genes and proteins.

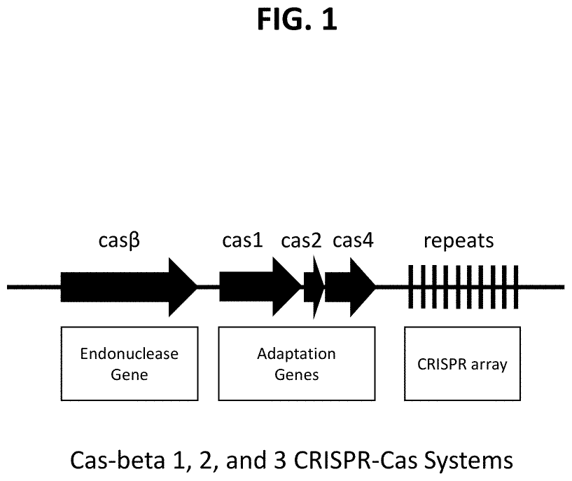

One distinguishing feature of the novel Cas-beta system is the locus architecture as described in FIG. 1. The order of polynucleotides, on the complementary strand, is as follows: a cas-beta gene, a cas1 gene, a cas2 gene, and an unclassified cas gene sharing low sequence identity with cas4. Following the unclassified cas gene is a CRISPR array comprising 37 repeats of a 37 nucleotide sequence, 33 of which are identical, three of which are at 97.3% identity, and the terminal one at 78.4% identity. The spacer polynucleotides between each array repeat are 35 or 36 nucleotides long.

### CRISPR-Cas System Components

### Cas Proteins

A number of proteins may be encoded in the CRISPR cas operon, including those involved in adaptation (spacer insertion), interference (effector module target binding, target nicking or cleavage—e.g. endonuclease activity), expression (pre-crRNA processing), regulation, or other.

Two proteins, Cas1 and Cas2, are conserved among many CRISPR systems (for example, as described in Koonin et al., Curr Opinion Microbiology 37:67-78, 2017). Cas1 is a metal-dependent DNA-specific endonuclease that produces double-stranded DNA fragments. In some systems Cas1 forms a stable complex with Cas2, which is essential to spacer acquisition and insertion for CRISPR systems (Nunez et al., Nature Str Mol Biol 21:528-534, 2014).

A number of other proteins have been identified across different systems, including Cas4 (which may have similarity to a RecB nuclease) and is thought to play a role in the capture of new viral DNA sequences for incorporation into the CRISPR array (Zhang et al., PLOS One 7(10):e47232, 2012).

Some proteins may encompass a plurality of functions. For example, Cas9, the signature protein of Class 2 type II systems, has been demonstrated to be involved in pre-crRNA processing, target binding, as well as target cleavage.

The novel Cas-beta proteins disclosed herein include effector proteins (endonucleases) as well as adaptation proteins.

### Cas Endonucleases and Effectors

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases that have been described include, but are not limited to, for example:Cas3 (a feature of Class 1 type I systems), Cas9 (a feature of Class 2 type II systems) and Cas12 (Cpf1) (a feature of Class 2 type V systems).

Cas3 (and its variants Cas3′ and Cas3″) functions as a single-stranded DNA nuclease (HD domain) and an ATP-dependent helicase. A variant of the Cas3 endonuclease can be obtained by disabling the functional activity of one or both domains of the Cas3 endonuclease poly peptide. Disabling the ATPase dependent helicase activity (by deletion, knockout of the Cas3-helicase domain, or through mutagenesis of critical residues or by assembling the reaction in the absence of ATP as described previously (Sinkunas, T. et al, 2013, EMBO J. 32:385-394) can convert the cleavage ready Cascade comprising the modified Cas3 endonuclease into a nickase (as the HD domain is still functional). Disabling the HD endonuclease activity can be accomplished by any method known in the art, such as but not limited to, mutagenesis of critical residues of the HD domain, can convert the cleavage ready Cascade comprising the modified Cas3 endonuclease into a helicase. Disabling the both the Cas helicase and Cas3 HD endonuclease activity can be accomplished by any method known in the art, such as but not limited to, mutagenesis of critical residues of both the helicase and HD domains, can convert the cleavage ready Cascade comprising the modified Cas3 endonuclease into a binder protein that binds to a target sequence.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. Cas9 recognizes a 3′ GC-rich PAM sequence on the target dsDNA. A Cas9 protein comprises a RuvC nuclease with an HNH (H-N-H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

Cas12 (formerly referred to as Cpfl, and variants c2c1, c2c3, CasX, and CasY) comprise an RuvC nuclease domain and produced staggered, 5′ overhangs on the dsDNA target. Some variants do not require a tracrRNA, unlike the functionality of Cas9. Cas12 and its variants recognize a 5′ AT-rich PAM sequence on the target dsDNA. An insert domain, called Nuc, of the Cas12a protein has been demonstrated to be responsible for target strand cleavage (Yamano et al., Cell 2016, 165:949-962). Additional mutation studies in other Cas12 proteins demonstrated the Nuc domain contributes to guide and target binding, with the RuvC domain responsible for cleavage (Swarts et al., Mol Cell 2017, 66:221-233 e224).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

### Cas-Beta Endonucleases

A Cas-beta endonuclease is defined as a functional RNA-guided, PAM-dependent dsDNA cleavage protein of fewer than 900 amino acids, and comprises at least one of the motifs described in Table 1B.

RuvC domains have been demonstrated in the literature to encompass endonuclease functionality. A Cas-beta endonuclease may be isolated or identified from a locus that comprises a cas-beta gene encoding an effector protein, and an array comprising a plurality repeats. In some aspects, a cas-beta locus may further comprise a partial or whole cas1 gene, a cast gene, and/or a cas4 gene.

Zinc finger motifs are domains that coordinate one or more zinc ions, usually through Cysteine and Histidine sidechains, to stabilize their fold. Zinc fingers are named for the pattern of Cysteine and Histidine residues that coordinate the zinc ion (e.g., C4 means a zinc ion is coordinated by four Cysteine residues; C3H means a zinc ion is coordinated by three Cysteine residues and one Histidine residue).

Cas-beta endonucleases are RNA-guided endonucleases capable of binding to, and cleaving, a double-strand DNA target that comprises: (1) a sequence sharing homology with a nucleotide sequence of the guide RNA, and (2) a PAM sequence. In some aspects, the PAM is T-rich. In some aspects, the PAM is C-rich.

A Cas-beta endonuclease is functional as a double-strand-break-inducing agent, and may also be a nickase, or a single-strand-break inducing agent. In some aspects, a catalytically inactive Cas-beta endonuclease may be used to target or recruit to a target DNA sequence but not induce cleavage. In some aspects, a catalytically inactive Cas-beta protein may be used with a functional endonuclease, to cleave a target sequence. In some aspects, a catalytically inactive Cas-beta protein may be combined with a base editing molecule, such as a deaminase. In some aspects, a deaminase may be a cytidine deaminase. In some aspects, a deaminase may be an adenine deaminase. In some aspects, a deaminase may be ADAR-2.

A Cas-beta endonuclease is further defined as an RNA-guided double-strand DNA cleavage protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of any of SEW NOs: 13, 14, 15, or 57, or a functional fragment thereof, or functional variant thereof that retains at least partial activity. A “functional fragment” of a Cas-beta endonuclease retains the ability to recognize, or bind, or nick a single strand of a double-stranded polynucleotide, or cleave both strands of a double-stranded polynucleotide, or any combination of the preceding.

The Cas-beta endonuclease comprises one or more motifs described in Table 1B.

The Cas-beta endonuclease may be encoded by a polynucleotide that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, between 500 and 550, at least 600, between 600 and 650, at least 650, between 650 and 700, at least 700, between 700 and 750, at least 750, between 750 and 800, at least 800, between 800 and 850, at least 850, between 850 and 900, at least 900, between 900 and 950, at least 950, between 950 and 1000, at least 1000, or even greater than 1000 contiguous nucleotides of any of the sequences in the sequence listing.

A Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 06 March 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A Cas endonuclease can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically active and/or inactive Cas endonuclease can be fused to a heterologous sequence (US20140068797 published 6 Mar. 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A partially active or catalytically inactive Cas-beta endonuclease can also be fused to another protein or domain, for example Clo51 or Fokl nuclease, to generate double-strand breaks (Guilinger et al. Nature Biotechnology, volume 32, number 6, Jun. 2014).

A catalytically active or inactive Cas protein, such as the Cas-beta protein described herein, can also be in fusion with a molecule that directs editing of single or multiple bases in a polynucleotide sequence, for example a site-specific deaminase that can change the identity of a nucleotide, for example from C•G to T•A or an A•T to G•C (Gaudelli et al., Programmable base editing of A•T to G•C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A base editing fusion protein may comprise, for example, an active (double strand break creating), partially active (nickase) or deactivated (catalytically inactive) Cas-beta endonuclease and a deaminase (such as, but not limited to, a cytidine deaminase, an adenine deaminase, APOBEC1, APOBEC3A, BE2, BE3, BE4, ABEs, or the like). Base edit repair inhibitors and glycosylase inhibitors (e.g., uracil glycosylase inhibitor (to prevent uracil removal)) are contemplated as other components of a base editing system, in some embodiments.

The Cas endonucleases described herein can be expressed and purified by methods known in the art, for example as described in WO/2016/186953 published 24 Nov. 2016.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position. It is understood that based on the methods and embodiments described herein utilizing a novel guided Cas system one skilled in the art can now tailor these methods such that they can utilize any guided endonuclease system.

A Cas effector protein can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. The Cas endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region. Non-limiting examples of suitable NLS sequences herein include those disclosed in U.S. Pat. Nos. 6,660,830 and 7,309,576.

### Guide Polynucleotides

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. In some cases, there is a linker polynucleotide that connects the crRNA and tracrRNA to form a single guide, for example an sgRNA.

The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA- combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments, a crRNA molecule is selected from the group consisting of: SEW NOs: 22, 23, and 24.

In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA. The tracrRNA (trans-activating CRISPR RNA) comprises, in the 5′-to-3′ direction, (i) a sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015).

In some embodiments, a tracrRNA molecule is selected from the group consisting of: SEQID NOs: 25-30.

In one aspect, the guide polynucleotide is a guide polynucleotide capable of forming a PGEN as described herein, wherein said guide polynucleotide comprises a first nucleotide sequence domain that is complementary to a nucleotide sequence in a target DNA, and a second nucleotide sequence domain that interacts with said Cas endonuclease polypeptide.

In one aspect, the guide polynucleotide is a guide polynucleotide described herein, wherein the first nucleotide sequence and the second nucleotide sequence domain is selected from the group consisting of a DNA sequence, a RNA sequence, and a combination thereof.

In one aspect, the guide polynucleotide is a guide polynucleotide described herein, wherein the first nucleotide sequence and the second nucleotide sequence domain is selected from the group consisting of RNA backbone modifications that enhance stability, DNA backbone modifications that enhance stability, and a combination thereof (see Kanasty et al., 2013, Common RNA-backbone modifications, Nature Materials 12:976-977; US20150082478 published 19 March 2015 and US20150059010 published 26 Feb. 2015)

The guide RNA includes a dual molecule comprising a chimeric non-naturally occurring crRNA linked to at least one tracrRNA. A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. In some embodiments, an sgRNA molecule is selected from the group consisting of: SEW NOs: 31-36.

The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site. (US20150082478 published 19 Mar. 2015 and US20150059010 published 26 February 2015).

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limited to Xie et al. 2015, PNAS 112:3570-3575).

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

### Guide Polynucleotide/Cas Endonuclease Complexes

A guide polynucleotide/Cas endonuclease complex described herein is capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end -joining, NHEJ (prone to imperfect repair leading to mutations) or homologous recombination, HR. Each nick in these embodiments can be at least about 5, between 5 and 10, at least 10, between 10 and 15, at leastl5, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any integer between 5 and 100) bases apart from each other, for example. One or two Cas nickase proteins herein can be used in a Cas nickase pair. For example, a Cas9 nickase with a mutant RuvC domain, but functioning HNH domain (i.e., Cas9 HNH+/RuvC−), can be used (e.g., Streptococcus pyogenes Cas9 HNH+/RuvC−). Each Cas9 nickase (e.g., Cas9 HNH+/RuvC−) can be directed to specific DNA sites nearby each other (up to 100 base pairs apart) by using suitable RNA components herein with guide RNA sequences targeting each nickase to each specific DNA site.

A guide polynucleotide/Cas endonuclease complex in certain embodiments can bind to a DNA target site sequence, but does not cleave any strand at the target site sequence. Such a complex may comprise a Cas protein in which all of its nuclease domains are mutant, dysfunctional. For example, a Cas9 protein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

In one aspect, the guide polynucleotide/Cas endonuclease complex (PGEN) described herein is a PGEN, wherein said Cas endonuclease is optionally covalently or non-covalently linked, or assembled to at least one protein subunit, or functional fragment thereof.

In one embodiment of the disclosure, the guide polynucleotide/Cas endonuclease complex is a guide polynucleotide/Cas endonuclease complex (PGEN) comprising at least one guide polynucleotide and at least one Cas endonuclease polypeptide, wherein said Cas endonuclease polypeptide comprises at least one protein subunit, or a functional fragment thereof, wherein said guide polynucleotide is a chimeric non-naturally occurring guide polynucleotide, wherein said guide polynucleotide/Cas endonuclease complex is capable of recognizing, binding to, and optionally nicking, unwinding, or cleaving all or part of a target sequence.

The Cas effector protein can be a Cas-beta effector protein as disclosed herein.

In one embodiment of the disclosure, the guide polynucleotide/Cas effector complex is a guide polynucleotide/Cas effector protein complex (PGEN) comprising at least one guide polynucleotide and a Cas-beta effector protein, wherein said guide polynucleotide/Cas effector protein complex is capable of recognizing, binding to, and optionally nicking, unwinding, or cleaving all or part of a target sequence.

The PGEN can be a guide polynucleotide/Cas effector protein complex, wherein said Cas effector protein further comprises one copy or multiple copies of at least one protein subunit, or a functional fragment thereof. In some embodiments, said protein subunit is selected from the group consisting of a Cas1 protein subunit, a Cas2 protein subunit, a Cas4 protein subunit, and any combination thereof. The PGEN can be a guide polynucleotide/Cas effector protein complex, wherein said Cas effector protein further comprises at least two different protein subunits of selected from the group consisting of a Cas1, Cas2, and Cas4.

The PGEN can be a guide polynucleotide/Cas effector protein complex, wherein said Cas effector protein further comprises at least three different protein subunits, or functional fragments thereof, selected from the group consisting of Cas1, Cas2, and one additional Cas protein, optionally comprising Cas4.

In one aspect, the guide polynucleotide/Cas effector protein complex (PGEN) described herein is a PGEN, wherein said Cas effector protein is covalently or non-covalently linked to at least one protein subunit, or functional fragment thereof. The PGEN can be a guide polynucleotide/Cas effector protein complex, wherein said Cas effector protein polypeptide is covalently or non-covalently linked, or assembled to one copy or multiple copies of at least one protein subunit, or a functional fragment thereof, selected from the group consisting of a Casl protein subunit, a Cas2 protein subunit, a one additional Cas protein optionally comprising Cas4 protein subunit, and any combination thereof. The PGEN can be a guide polynucleotide/Cas effector protein complex, wherein said Cas effector protein is covalently or non-covalently linked or assembled to at least two different protein subunits selected from the group consisting of a Cas1, a Cas2, and one additional Cas protein, optionally comprising Cas4. The PGEN can be a guide polynucleotide/Cas effector protein complex, wherein said Cas effector protein is covalently or non-covalently linked to at least three different protein subunits, or functional fragments thereof, selected from the group consisting of a Cas1, a Cas2, and one additional Cas protein, optionally comprising Cas4, and any combination thereof.

Any component of the guide polynucleotide/Cas effector protein complex, the guide polynucleotide/Cas effector protein complex itself, as well as the polynucleotide modification template(s) and/or donor DNA(s), can be introduced into a heterologous cell or organism by any method known in the art.

Recombinant Constructs for Transformation of Cells

The disclosed guide polynucleotides, Cas endonucleases, polynucleotide modification templates, donor DNAs, guide polynucleotide/Cas endonuclease systems disclosed herein, and any one combination thereof, optionally further comprising one or more polynucleotide(s) of interest, can be introduced into a cell. Cells include, but are not limited to, human, non-human, animal, bacterial, fungal, insect, yeast, non-conventional yeast, and plant cells as well as plants and seeds produced by the methods described herein.

Standard recombinant DNA and molecular cloning techniques used herein are well known in the art and are described more fully in Sambrook et al.,Molecular Cloning:A Laboratory Manual; Cold Spring Harbor Laboratory: Cold Spring Harbor, N.Y. (1989). Transformation methods are well known to those skilled in the art and are described infra.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

Components for Expression and Utilization of Novel CRISPR-Cas Systems in Prokaryotic and Eukaryotic cells

The invention further provides expression constructs for expressing in a prokaryotic or eukaryotic cell/organism a guide RNA/Cas system that is capable of recognizing, binding to, and optionally nicking, unwinding, or cleaving all or part of a target sequence.

In one embodiment, the expression constructs of the disclosure comprise a promoter operably linked to a nucleotide sequence encoding a Cas gene (or plant optimized, including a Cas endonuclease gene described herein) and a promoter operably linked to a guide RNA of the present disclosure. The promoter is capable of driving expression of an operably linked nucleotide sequence in a prokaryotic or eukaryotic cell/organism.

Nucleotide sequence modification of the guide polynucleotide, VT domain and/or CER domain can be selected from, but not limited to , the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking , a modification or sequence that provides a binding site for proteins , a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-0-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41:4336-4343; Ma et al., Mol. Ther. Nucleic Acids 3:e161). This strategy has been successfully applied in cells of several different species including maize and soybean (US20150082478 published 19 Mar. 2015). Methods for expressing RNA components that do not have a 5′ cap have been described (WO2016/025131 published 18 Feb. 2016).

Various methods and compositions can be employed to obtain a cell or organism having a polynucleotide of interest inserted in a target site for a Cas endonuclease. Such methods can employ homologous recombination (HR) to provide integration of the polynucleotide of interest at the target site. In one method described herein, a polynucleotide of interest is introduced into the organism cell via a donor DNA construct.

The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity at least of about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning:A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, N.Y.).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

### Polynucleotides of Interest

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

General categories of polynucleotides of interest include, for example, genes of interest involved in information, such as zinc fingers, those involved in communication, such as kinases, and those involved in housekeeping, such as heat shock proteins. More specific polynucleotides of interest include, but are not limited to, genes involved in traits of agronomic interest such as but not limited to:crop yield, grain quality, crop nutrient content, starch and carbohydrate quality and quantity as well as those affecting kernel size, sucrose loading, protein quality and quantity, nitrogen fixation and/or utilization, fatty acid and oil composition, genes encoding proteins conferring resistance to abiotic stress (such as drought, nitrogen, temperature, salinity, toxic metals or trace elements, or those conferring resistance to toxins such as pesticides and herbicides), genes encoding proteins conferring resistance to biotic stress (such as attacks by fungi, viruses, bacteria, insects, and nematodes, and development of diseases associated with these organisms).

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS, also referred to as acetohydroxyacid synthase, AHAS), in particular the sulfonylurea (UK:sulphonylurea) type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and 9,187,762. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that comprises it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as sulphonylureas, glufosinate ammonium, bromoxynil, imidazolinones, and 2,4-dichlorophenoxyacetate (2,4-D). See for example, Acetolactase synthase (ALS) for resistance to sulfonylureas, imidazolinones, triazolopyrimidine sulfonamides, pyrimidinylsalicylates and sulphonylaminocarbonyl-triazolinones (Shaner and Singh, 1997, Herbicide Activity: Toxicol Biochem Mol Biol 69-110); glyphosate resistant 5-enolpyruvylshikimate-3-phosphate (EPSPS) (Saroha et al. 1998, J. Plant Biochemistry & Biotechnology Vol 7:65-72);

Polynucleotides of interest includes genes that can be stacked or used in combination with other traits, such as but not limited to herbicide resistance or any other trait described herein. Polynucleotides of interest and/or traits can be stacked together in a complex trait locus as described in US20130263324 published 3 Oct. 2013 and in WO/2013/112686, published 1 Aug. 2013.

A polypeptide of interest includes any protein or polypeptide that is encoded by a polynucleotide of interest described herein.

Further provided are methods for identifying at least one plant cell, comprising in its genome, a polynucleotide of interest integrated at the target site. A variety of methods are available for identifying those plant cells with insertion into the genome at or near to the target site. Such methods can be viewed as directly analyzing a target sequence to detect any change in the target sequence, including but not limited to PCR methods, sequencing methods, nuclease digestion, Southern blots, and any combination thereof. See, for example, US20090133152 published 21 May 2009. The method also comprises recovering a plant from the plant cell comprising a polynucleotide of interest integrated into its genome. The plant may be sterile or fertile. It is recognized that any polynucleotide of interest can be provided, integrated into the plant genome at the target site, and expressed in a plant.

### Optimization of Sequences for Expression in Plants

Methods are available in the art for synthesizing plant-preferred genes. See, for example, U.S. Pat. Nos. 5,380,831, and 5,436,391, and Murray et al. (1989) Nucleic Acids Res. 17:477-498. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

### Expression Elements

Any polynucleotide encoding a Cas protein or other CRISPR system component disclosed herein may be functionally linked to a heterologous expression element, to facilitate transcription or regulation in a host cell. Such expression elements include but are not limited to:promoter, leader, intron, and terminator. Expression elements may be “minimal”—meaning a shorter sequence derived from a native source, that still functions as an expression regulator or modifier. Alternatively, an expression element may be “optimized”—meaning that its polynucleotide sequence has been altered from its native state in order to function with a more desirable characteristic in a particular host cell (for example, but not limited to, a bacterial promoter may be “maize-optimized” to improve its expression in corn plants). Alternatively, an expression element may be “synthetic”—meaning that it is designed in silico and synthesized for use in a host cell. Synthetic expression elements may be entirely synthetic, or partially synthetic (comprising a fragment of a naturally-occurring polynucleotide sequence).

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels.

A plant promoter includes a promoter capable of initiating transcription in a plant cell. For a review of plant promoters, see, Potenza et al., 2004, In vitro Cell Dev Biol 40:1-22; Porto et al., 2014, Molecular Biotechnology (2014), 56(1), 38-49.

Constitutive promoters include, for example, the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; ALS promoter (U.S. Pat. No. 5,659,026) and the like.

Tissue-preferred promoters can be utilized to target enhanced expression within a particular plant tissue. Tissue-preferred promoters include, for example, WO2013103367 published 11 July 2013, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J4:495-505. Leaf-preferred promoters include, for example, Yamamoto et al., (1997) Plant J12:255-65; Kwon et al., (1994) Plant Physiol 105:357-67; Yamamoto et al., (1994) Plant Cell Physiol 35:773-8; Gotor et al., (1993) Plant J3:509-18; Orozco et al., (1993) Plant Mol Biol 23:1129-38; Matsuoka et al., (1993) Proc. Natl. Acad. Sci. USA 90:9586-90; Simpson et al., (1958) EMBO J4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes rolC and rolD root-inducing genes); Teeri et al, (1989) EMBO J 8:343-50 (Agrobacterium wound-induced TR1′ and TR2′ genes); VfENOD-GRP3 gene promoter (Kuster et al, (1995) Plant Mol Biol 29:759-72); and rolB promoter (Capana et al, (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Ciml (cytokinin-induced message); cZ19B1 (maize 19 kDa zein); and milps (myo-inositol-1-phosphate synthase); and for example those disclosed in WO2000011177 published 2 Mar. 2000 and U.S. Pat. 6,225,529. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nucl. See also, WO2000012733 published 09 March 2000, where seed-preferred promoters from END1 and END2 genes are disclosed.

Chemical inducible (regulated) promoters can be used to modulate the expression of a gene in a prokaryotic and eukaryotic cell or organism through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-II-27, WO1993001294 published 21 Jan. 1993), activated by hydrophobic electrophilic compounds used as pre-emergent herbicides, and the tobacco PR-1a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Pathogen inducible promoters induced following infection by a pathogen include, but are not limited to those regulating expression of PR proteins, SAR proteins, beta-1,3-glucanase, chitinase, etc.

A stress-inducible promoter includes the RD29A promoter (Kasuga et al. (1999) Nature Biotechnol. 17:287-91). One of ordinary skill in the art is familiar with protocols for simulating stress conditions such as drought, osmotic stress, salt stress and temperature stress and for evaluating stress tolerance of plants that have been subjected to simulated or naturally-occurring stress conditions.

Another example of an inducible promoter useful in plant cells, is the ZmCAS1 promoter, described in US20130312137 published 21 November 2013.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, N.Y.:Academic Press), pp. 1-82.

Modification of Genomes with Novel CRISPR-Cas System Components

As described herein, a guided Cas endonuclease can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92). In maize protoplasts, experiments with linear DNA molecules demonstrated enhanced homologous recombination between plasmids (Lyznik et al., (1991) Mol Gen Genet 230:209-18).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

Alteration of the genome of a prokaryotic and eukaryotic cell or organism cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28: e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., Scientific American Books distributed by WH Freeman & Co.).

### Gene Targeting

The guide polynucleotide/Cas systems described herein can be used for gene targeting.

In general, DNA targeting can be performed by cleaving one or both strands at a specific polynucleotide sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example:US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with a Cas endonuclease and complexes with a Cas endonuclease and a guide polynucleotide. Following characterization of the guide RNA and PAM sequence, components of the endonuclease and associated CRISPR RNA (crRNA) may be utilized to modify chromosomal DNA in other organisms including plants. To facilitate optimal expression and nuclear localization (for eukaryotic cells), the genes comprising the complex may be optimized as described in WO2016186953 published 24 Nov. 2016, and then delivered into cells as DNA expression cassettes by methods known in the art. The components necessary to comprise an active complex may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 April 2017), or any combination thereof. Additionally, a part or part(s) of the complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the complex to its DNA target site, as described, for example, in WO2017105991 published 22 Jun. 2017. Nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage complex results in degradation of the randomized PAM library, the complex can be converted into a nickase by disabling the ATPase dependent helicase activity either through mutagenesis of critical residues or by assembling the reaction in the absence of ATP as described previously (Sinkunas, T. et al, 2013, EMBO J. 32:385-394). Two regions of PAM randomization separated by two protospacer targets may be utilized to generate a double-stranded DNA break which may be captured and sequenced to examine the PAM sequences that support cleavage by the respective complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, the chemical alteration of at least one nucleotide, and (v) any combination of (i)-(iv).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning:A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, N.Y.).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J. 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 March 2015 and WO2015026886 published 26 February 2015).

Polynucleotides of interest and/or traits can be stacked together in a complex trait locus as described in WO2012129373 published 27 September 2012, and in WO2013112686, published 01 August 2013. The guide polynucleotide/Cas9 endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 September 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 March 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example:US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

Introduction of CRISPR-Cas System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing , sexual breeding, and any combination thereof.

For example, the guide polynucleotide (guide RNA, crNucleotide +tracrNucleotide, guide DNA and/or guide RNA-DNA molecule) can be introduced into a cell directly (transiently) as a single stranded or double stranded polynucleotide molecule. The guide RNA (or crRNA +tracrRNA) can also be introduced into a cell indirectly by introducing a recombinant DNA molecule comprising a heterologous nucleic acid fragment encoding the guide RNA (or crRNA +tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3:e161; DiCarlo et al, 2013, Nucleic Acids Res. 41:4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

Plant cells differ from animal cells (such as human cells), fungal cells (such as yeast cells) and protoplasts, including for example plant cells comprise a plant cell wall which may act as a barrier to the delivery of components.

Delivery of the Cas endonuclease, and/or the guide RNA, and/or a ribonucleoprotein complex, and/or a polynucleotide encoding any one or more of the preceding, into plant cells can be achieved through methods known in the art, for example but not limited to: Rhizobiales-mediated transformation (e.g., Agrobacterium, Ochrobactrum), particle mediated delivery (particle bombardment), polyethylene glycol (PEG)-mediated transfection (for example to protoplasts), electroporation, cell-penetrating peptides, or mesoporous silica nanoparticle (MSN)-mediated direct protein delivery.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 April 2017.

Introducing a guide RNA/Cas endonuclease complex described herein, (representing the cleavage ready complex described herein) into a cell includes introducing the individual components of said complex either separately or combined into the cell, and either directly (direct delivery as RNA for the guide and protein for the Cas endonuclease and protein subunits, or functional fragments thereof) or via recombination constructs expressing the components (guide RNA, Cas endonuclease, protein subunits, or functional fragments thereof). Introducing a guide RNA/Cas endonuclease complex (RGEN) into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one protein subunit) can be assembled in vitro or assembled by any means known in the art prior to being introduced into a cell (targeted for genome modification as described herein).

Direct delivery of the RGEN ribonucleoprotein, allows for genome editing at a target site in the genome of a cell which can be followed by rapid degradation of the complex, and only a transient presence of the complex in the cell. This transient presence of the RGEN complex may lead to reduced off-target effects. In contrast, delivery of RGEN components (guide RNA, Cas9 endonuclease) via plasmid DNA sequences can result in constant expression of RGENs from these plasmids which can intensify off target effects (Cradick, T. J. et al. (2013) Nucleic Acids Res 41:9584-9592; Fu, Yet al. (2014) Nat. Biotechnol. 31:822-826).

Direct delivery can be achieved by combining any one component of the guide RNA/Cas endonuclease complex (RGEN), representing the cleavage ready complex described herein, (such as at least one guide RNA, at least one Cas protein, and optionally one additional protein), with a delivery matrix comprising a microparticle (such as but not limited to of a gold particle, tungsten particle, and silicon carbide whisker particle) (see also WO2017070032 published 27 Apr. 2017). The delivery matrix may comprise any one of the components, such as the Cas endonuclease, that is attached to a solid matrix (e.g., a particle for bombardment).

In one aspect the guide polynucleotide/Cas endonuclease complex, is a complex wherein the guide RNA and Cas endonuclease protein forming the guide RNA/Cas endonuclease complex are introduced into the cell as RNA and protein, respectively.

In one aspect the guide polynucleotide/Cas endonuclease complex, is a complex wherein the guide RNA and Cas endonuclease protein and the at least one protein subunit of a complex forming the guide RNA/Cas endonuclease complex are introduced into the cell as RNA and proteins, respectively.

In one aspect the guide polynucleotide/Cas endonuclease complex, is a complex wherein the guide RNA and Cas endonuclease protein and the at least one protein subunit of a complex forming the guide RNA/Cas endonuclease complex (cleavage ready complex) are preassembled in vitro and introduced into the cell as a ribonucleotide-protein complex.

Protocols for introducing polynucleotides, polypeptides or polynucleotide-protein complexes (PGEN, RGEN) into eukaryotic cells, such as plants or plant cells are known and include microinjection (Crossway et al, (1986) Biotechniques 4:320-34 and U.S. Patent No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s):Gerhardt, Rosario. Publisher:InTech, Rijeka, Croatia. CODEN:69PQBP; ISBN:978-953-307-201-2), direct gene transfer (Paszkowski et al., (1984) EMBO J3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture:Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, N.Y.), pp. 197-209 (pollen); Kaeppler et al., (1990) Plant Cell Rep 9:415-8) and Kaeppler et al., (1992) Theor Appl Genet 84:560-6 (whisker-mediated transformation); D′Halluin et al., (1992) Plant Cell 4:1495-505 (electroporation); Li et al., (1993) Plant Cell Rep 12:250-5; Christou and Ford (1995) Annals Botany 75:407-13 (rice) and Osj oda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Cells and Plants

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to:oilseed rape or Canola) (Brassica napus, B. campestris, Brassica rapa, Brassica. juncea), alfalfa (Medicago sativa),), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum).

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifoha), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

A non-limiting example of how two traits can be stacked into the genome at a genetic distance of, for example, 5 cM from each other is described as follows:A first plant comprising a first transgenic target site integrated into a first DSB target site within the genomic window and not having the first genomic locus of interest is crossed to a second transgenic plant, comprising a genomic locus of interest at a different genomic insertion site within the genomic window and the second plant does not comprise the first transgenic target site. About 5% of the plant progeny from this cross will have both the first transgenic target site integrated into a first DSB target site and the first genomic locus of interest integrated at different genomic insertion sites within the genomic window. Progeny plants having both sites in the defined genomic window can be further crossed with a third transgenic plant comprising a second transgenic target site integrated into a second DSB target site and/or a second genomic locus of interest within the defined genomic window and lacking the first transgenic target site and the first genomic locus of interest. Progeny are then selected having the first transgenic target site, the first genomic locus of interest and the second genomic locus of interest integrated at different genomic insertion sites within the genomic window. Such methods can be used to produce a transgenic plant comprising a complex trait locus having at least 1, 2, 3, 4, 5, 6, 7, 8, 9, 10, 11, 12, 13, 14,15, 16, 17, 19, 19, 20, 21, 22, 23, 24, 25, 26, 27, 28, 29, 30, 31 or more transgenic target sites integrated into DSB target sites and/or genomic loci of interest integrated at different sites within the genomic window. In such a manner, various complex trait loci can be generated.

### Cells and Animals

The presently disclosed polynucleotides and polypeptides can be introduced into an animal cell. Animal cells can include, but are not limited to: an organism of a phylum including chordates, arthropods, mollusks, annelids, cnidarians, or echinoderms; or an organism of a class including mammals, insects, birds, amphibians, reptiles, or fishes. In some aspects, the animal is human, mouse, C. elegans, rat, fruit fly (Drosophila spp.), zebrafish, chicken, dog, cat, guinea pig, hamster, chicken, Japanese ricefish, sea lamprey, pufferfish, tree frog (e.g., Xenopus spp.), monkey, or chimpanzee. Particular cell types that are contemplated include haploid cells, diploid cells, reproductive cells, neurons, muscle cells, endocrine or exocrine cells, epithelial cells, muscle cells, tumor cells, embryonic cells, hematopoietic cells, bone cells, germ cells, somatic cells, stem cells, pluripotent stem cells, induced pluripotent stem cells, progenitor cells, meiotic cells, and mitotic cells. In some aspects, a plurality of cells from an organism may be used.

The novel Cas9 orthologs disclosed may be used to edit the genome of an animal cell in various ways. In one aspect, it may be desirable to delete one or more nucleotides. In another aspect, it may be desirable to insert one or more nucleotides. In one aspect, it may be desirable to replace one or more nucleotides. In another aspect, it may be desirable to modify one or more nucleotides via a covalent or non-covalent interaction with another atom or molecule.

Genome modification via a Cas9 ortholog may be used to effect a genotypic and/or phenotypic change on the target organism. Such a change is preferably related to an improved phenotype of interest or a physiologically-important characteristic, the correction of an endogenous defect, or the expression of some type of expression marker. In some aspects, the phenotype of interest or physiologically-important characteristic is related to the overall health, fitness, or fertility of the animal, the ecological fitness of the animal, or the relationship or interaction of the animal with other organisms in its environment. In some aspects, the phenotype of interest or physiologically-important characteristic is selected from the group consisting of: improved general health, disease reversal, disease modification, disease stabilization, disease prevention, treatment of parasitic infections, treatment of viral infections, treatment of retroviral infections, treatment of bacterial infections, treatment of neurological disorders (for example but not limited to: multiple sclerosis), correction of endogenous genetic defects (for example but not limited to: metabolic disorders, Achondroplasia, Alpha-1 Antitrypsin Deficiency, Antiphospholipid Syndrome, Autism, Autosomal Dominant Polycystic Kidney Disease, Barth syndrome, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Turner Syndrome, Velocardiofacial Syndrome, WAGR Syndrome, and Wilson Disease), treatment of innate immune disorders (for example but not limited to: immunoglobulin subclass deficiencies), treatment of acquired immune disorders (for example but not limited to: AIDS and other HIV-related disorders), treatment of cancer, as well as treatment of diseases, including rare or “orphan” conditions, that have eluded effective treatment options with other methods.

Cells that have been genetically modified using the compositions or methods disclosed herein may be transplanted to a subject for purposes such as gene therapy, e.g. to treat a disease, or as an antiviral, antipathogenic, or anticancer therapeutic, for the production of genetically modified organisms in agriculture, or for biological research.

In vitro Polynucleotide Detection, Binding, and Modification

The compositions disclosed herein may further be used as compositions for use in in vitro methods, in some aspects with isolated polynucleotide sequence(s). Said isolated polynucleotide sequence(s) may comprise one or more target sequence(s) for modification. In some aspects, said isolated polynucleotide sequence(s) may be genomic DNA, a PCR product, or a synthesized oligonucleotide.

### Compositions

Modification of a target sequence may be in the form of a nucleotide insertion, a nucleotide deletion, a nucleotide substitution, the addition of an atom molecule to an existing nucleotide, a nucleotide modification, or the binding of a heterologous polynucleotide or polypeptide to said target sequence. The insertion of one or more nucleotides may be accomplished by the inclusion of a donor polynucleotide in the reaction mixture: said donor polynucleotide is inserted into a double-strand break created by said Cas-beta ortholog polypeptide. The insertion may be via non-homologous end joining or via homologous recombination.

In one aspect, the sequence of the target polynucleotide is known prior to modification, and compared to the sequence(s) of polynucleotide(s) that result from treatment with the Cas-beta ortholog. In one aspect, the sequence of the target polynucleotide is not known prior to modification, and the treatment with the Cas-beta ortholog is used as part of a method to determine the sequence of said target polynucleotide.

Polynucleotide modification with a Cas-beta ortholog may be accomplished by usage of a full-length polypeptide identified from a Cas locus, or from a fragment, modification, or variant of a polypeptide identified from a Cas locus. In some aspects, said Cas-beta ortholog is obtained or derived from Armatimonadetes bacterium. In some aspects, said Cas-beta ortholog is a polypeptide sharing at least 80% identity with any of SEQID NOs:13-15. In some aspects, said Cas-beta ortholog is a functional variant of any of SEQID NOs:13-15. In some aspects, said Cas-beta ortholog is a functional fragment of any of SEQID NOs:13-15. In some aspects, said Cas-beta ortholog is a Cas-beta polypeptide encoded by a polynucleotide selected from the group consisting of: SEQID NO:13-15. In some aspects, said Cas-beta ortholog is a Cas-beta polypeptide that recognizes a PAM sequence listed in any of Tables 4-83. In some aspects, said Cas-beta ortholog is a Cas-beta polypeptide identified from an organism listed in the sequence listing.

In some aspects, the Cas-beta ortholog is provided as a Cas-beta polynucleotide. In some aspects, said Cas-beta polynucleotide is selected from the group consisting of: SEQID NO:1-85, or a sequence sharing at least 80%, 85%, 90%, 95%, 97%, 99%, or 100% with any one of SEQID NO:10-12.

In some aspects, the Cas-beta ortholog may be selected from the group consisting of: an unmodified wild type Cas-beta ortholog, a functional Cas-beta ortholog variant, a functional Cas-beta ortholog fragment, a fusion protein comprising an active or deactivated Cas-beta ortholog, a Cas-beta ortholog further comprising one or more nuclear localization sequences (NLS) on the C-terminus or on the N-terminus or on both the N- and C-termini, a biotinylated Cas-beta ortholog, a Cas-beta ortholog nickase, a Cas-beta ortholog endonuclease, a Cas-beta ortholog further comprising a Histidine tag, and a mixture of any two or more of the preceding.

In some aspects, the Cas-beta ortholog is a fusion protein further comprising a nuclease domain, a transcriptional activator domain, a transcriptional repressor domain, an epigenetic modification domain, a cleavage domain, a nuclear localization signal, a cell-penetrating domain, a translocation domain, a marker, or a transgene that is heterologous to the target polynucleotide sequence or to the cell from which said target polynucleotide sequence is obtained or derived.

In some aspects, a plurality of Cas-beta orthologs may be desired. In some aspects, said plurality may comprise Cas-beta orthologs derived from different source organisms or from different loci within the same organism. In some aspects, said plurality may comprise Cas-beta orthologs with different binding specificities to the target polynucleotide. In some aspects, said plurality may comprise Cas-beta orthologs with different cleavage efficiencies. In some aspects, said plurality may comprise Cas-beta orthologs with different PAM specificities. In some aspects, said plurality may comprise orthologs of different molecular compositions, i.e., a polynucleotide Cas-beta ortholog and a polypeptide Cas-beta ortholog.

The guide polynucleotide may be provided as a single guide RNA (sgRNA), a chimeric molecule comprising a tracrRNA, a chimeric molecule comprising a crRNA, a chimeric RNA-DNA molecule, a DNA molecule, or a polynucleotide comprising one or more chemically modified nucleotides.

The storage conditions of the Cas-beta ortholog and/or the guide polynucleotide include parameters for temperature, state of matter, and time. In some aspects, the Cas-beta ortholog and/or the guide polynucleotide is stored at about −80 degrees Celsius, at about −20 degrees Celsius, at about 4 degrees Celsius, at about 20-25 degrees Celsius, or at about 37 degrees Celsius. In some aspects, the Cas-beta ortholog and/or the guide polynucleotide is stored as a liquid, a frozen liquid, or as a lyophilized powder. In some aspects, the Cas-beta ortholog and/or the guide polynucleotide is stable for at least one day, at least one week, at least one month, at least one year, or even greater than one year.

Any or all of the possible polynucleotide components of the reaction (e.g., guide polynucleotide, donor polynucleotide, optionally a Cas-beta polynucleotide) may be provided as part of a vector, a construct, a linearized or circularized plasmid, or as part of a chimeric molecule. Each component may be provided to the reaction mixture separately or together. In some aspects, one or more of the polynucleotide components are operably linked to a heterologous noncoding regulatory element that regulates its expression.

The method for modification of a target polynucleotide comprises combining the minimal elements into a reaction mixture comprising: a Cas-beta ortholog (or variant, fragment, or other related molecule as described above), a guide polynucleotide comprising a sequence that is substantially complementary to, or selectively hybridizes to, the target polynucleotide sequence of the target polynucleotide, and a target polynucleotide for modification. In some aspects, the Cas-beta ortholog is provided as a polypeptide. In some aspects, the Cas-beta ortholog is provided as a Cas-beta ortholog polynucleotide. In some aspects, the guide polynucleotide is provided as an RNA molecule, a DNA molecule, an RNA:DNA hybrid, or a polynucleotide molecule comprising a chemically-modified nucleotide.

The storage buffer of any one of the components, or the reaction mixture, may be optimized for stability, efficacy, or other parameters. Additional components of the storage buffer or the reaction mixture may include a buffer composition, Tris, EDTA, dithiothreitol (DTT), phosphate-buffered saline (PBS), sodium chloride, magnesium chloride, HEPES, glycerol, BSA, a salt, an emulsifier, a detergent, a chelating agent, a redox reagent, an antibody, nuclease-free water, a proteinase, and/or a viscosity agent. In some aspects, the storage buffer or reaction mixture further comprises a buffer solution with at least one of the following components: HEPES, MgC12, NaC1, EDTA, a proteinase, Proteinase K, glycerol, nuclease-free water.

Incubation conditions will vary according to desired outcome. The temperature is preferably at least 10 degrees Celsius, between 10 and 15, at least 15, between 15 and 17, at least 17, between 17 and 20, at least 20, between 20 and 22, at least 22, between 22 and 25, at least 25, between 25 and 27, at least 27, between 27 and 30, at least 30, between 30 and 32, at least 32, between 32 and 35, at least 35, at least 36, at least 37, at least 38, at least 39, at least 40, or even greater than 40 degrees Celsius. The time of incubation is at least 1 minute, at least 2 minutes, at least 3 minutes, at least 4 minutes, at least 5 minutes, at least 6 minutes, at least 7 minutes, at least 8 minutes, at least 9 minutes, at least 10 minutes, or even greater than 10 minutes.

The sequence(s) of the polynucleotide(s) in the reaction mixture prior to, during, or after incubation may be determined by any method known in the art. In one aspect, modification of a target polynucleotide may be ascertained by comparing the sequence(s) of the polynucleotide(s) purified from the reaction mixture to the sequence of the target polynucleotide prior to combining with the Cas-beta ortholog.

Any one or more of the compositions disclosed herein, useful for in vitro or in vivo polynucleotide detection, binding, and/or modification, may be comprised within a kit. A kit comprises a Cas-beta ortholog or a polynucleotide Cas-beta ortholog encoding such, optionally further comprising buffer components to enable efficient storage, and one or more additional compositions that enable the introduction of said Cas-beta ortholog or Cas-beta ortholog to a heterologous polynucleotide, wherein said Cas-beta ortholog or Cas-beta ortholog is capable of effecting a modification, addition, deletion, or substitution of at least one nucleotide of said heterologous polynucleotide. In an additional aspect, a Cas-beta ortholog disclosed herein may be used for the enrichment of one or more polynucleotide target sequences from a mixed pool. In an additional aspect, a Cas-beta ortholog disclosed herein may be immobilized on a matrix for use in in vitro target polynucleotide detection, binding, and/or modification.

A Cas-beta endonuclease may be attached, associated with, or affixed to a solid matrix for the purposes of storage, purification, and/or characterization. Examples of a solid matrix include, but are not limited to: a filter, a chromatography resin, an assay plate, a test tube, a cryogenic vial, etc. A Cas-beta endonuclease may be substantially purified and stored in an appropriate buffer solution, or lyophilized.

### Methods of Detection

Methods of detecting the Cas-beta:guide polynucleotide complex bound to the target polynucleotide may include any known in the art, including but not limited to microscopy, chromatographic separation, electrophoresis, immunoprecipitation, filtration, nanopore separation, microarrays, as well as those described below.

A DNA Electrophoretic Mobility Shift Assay (EMSA): studies proteins binding to known DNA oligonucleotide probes and assesses the specificity of the interaction. The technique is based on the principle that protein-DNA complexes migrate more slowly than free DNA molecules when subjected to polyacrylamide or agarose gel electrophoresis. Because the rate of DNA migration is retarded upon protein binding, the assay is also called a gel retardation assay. Adding a protein-specific antibody to the binding components creates an even larger complex (antibody-protein-DNA) which migrates even slower during electrophoresis, this is known as a supershift and can be used to confirm protein identities.

DNA Pull-down Assays use a DNA probe labelled with a high affinity tag, such as biotin, which allows the probe to be recovered or immobilized. A DNA probe can be complexed with a protein from a cell lysate in a reaction similar to that used in the EMSA and then used to purify the complex using agarose or magnetic beads. The proteins are then eluted from the DNA and detected by Western blot or identified by mass spectrometry. Alternatively, the protein may be labelled with an affinity tag or the DNA-protein complex may be isolated using an antibody against the protein of interest (similar to a supershift assay). In this case, the unknown DNA sequence bound by the protein is detected by Southern blotting or through PCR analysis.

Reporter assays provide a real-time in vivo read-out of translational activity for a promoter of interest. Reporter genes are fusions of a target promoter DNA sequence and a reporter gene DNA sequence which is customized by the researcher and the DNA sequence codes for a protein with detectable properties like firefly/Renilla luciferase or alkaline phosphatase. These genes produce enzymes only when the promoter of interest is activated. The enzyme, in turn, catalyses a substrate to produce either light or a colour change that can be detected by spectroscopic instrumentation. The signal from the reporter gene is used as an indirect determinant for the translation of endogenous proteins driven from the same promoter.

Microplate Capture and Detection Assays use immobilized DNA probes to capture specific protein-DNA interactions and confirm protein identities and relative amounts with target specific antibodies. Typically, a DNA probe is immobilized on the surface of 96- or 384-well microplates coated with streptavidin. A cellular extract is prepared and added to allow the binding protein to bind to the oligonucleotide. The extract is then removed and each well is washed several times to remove non-specifically bound proteins. Finally, the protein is detected using a specific antibody labelled for detection. This method can be extremely sensitive , detecting less than 0.2 pg of the target protein per well. This method may also be utilized for oligonucleotides labelled with other tags, such as primary amines that can be immobilized on microplates coated with an amine-reactive surface chemistry.

DNA Footprinting is one of the most widely used methods for obtaining detailed information on the individual nucleotides in protein—DNA complexes, even inside living cells. In such an experiment, chemicals or enzymes are used to modify or digest the DNA molecules. When sequence specific proteins bind to DNA they can protect the binding sites from modification or digestion. This can subsequently be visualized by denaturing gel electrophoresis, where unprotected DNA is cleaved more or less at random. Therefore it appears as a ‘ladder’ of bands and the sites protected by proteins have no corresponding bands and look like foot prints in the pattern of bands. The foot prints there by identify specific nucleosides at the protein—DNA binding sites.

Microscopic techniques include optical, fluorescence, electron, and atomic force microscopy (AFM).

Chromatin immunoprecipitation analysis (ChIP) causes proteins to bind covalently to their DNA targets, after which they are unlinked and characterized separately.

Systematic Evolution of Ligands by EXponential enrichment (SELEX) exposes target proteins to a random library of oligonucleotides. Those genes that bind are separated and amplified by PCR.

The following aspects, without limitation, are among those contemplated:

Aspect 1: A synthetic composition comprising: (a) a CRISPR-Cas effector protein comprising: one positively charged bridge-helix-like domain, one bridge-helix domain, a DNA binding domain optionally comprising a helix-turn-helix motif, an RuvC-I domain, an RuvC-II domain, and an RuvC-III domain, and (b) a heterologous polynucleotide; wherein said effector protein does not comprise an HNH domain adjacent to the RuvC-II domain, wherein said effector protein comprises at least one motif selected from Table 1B.

Aspect 2: A synthetic composition comprising: (a) a CRISPR-Cas effector protein comprising an RuvC-I domain, and at least one domain upstream of the RuvC-I domain selected from the group consisting of: a positively-charged bridge-helix-like domain, a DNA binding domain optionally comprising a helix-turn-helix motif, and a combination thereof; and

(b) a heterologous polynucleotide; wherein said effector protein does not comprise an HNH domain adjacent to the RuvC-II domain, wherein said effector protein comprises at least one motif selected from Table 1B.

Aspect 3: The synthetic composition of Aspect 1, wherein said CRISPR-Cas effector protein comprises a plurality of domains in this order: a positively-charged bridge-helix-like domain, a DNA binding domain optionally comprising a helix-turn-helix motif, an RuvC-I domain, a bridge-helix domain, an RuvC-II domain, and an RuvC-III domain; wherein said effector protein does not comprise an HNH domain adjacent to the RuvC-II domain.

Aspect 4: A synthetic composition comprising: (a) a CRISPR-Cas effector protein derived from Armatimonadetes, and (b) a heterologous polynucleotide; wherein said effector protein comprises at least one motif selected from Table 1B.

Aspect 5: A synthetic composition comprising a Cas endonuclease, wherein the Cas endonuclease comprises at least one motif selected from Table 1B.

Aspect 6: The synthetic composition of any of Aspects 1-4, wherein said heterologous polynucleotide is (a) derived from an organism of different taxonomy than that from which the effector protein was derived, (b) a different target than the natural target of the effector protein, or (c) completely or partially artificial.

Aspect 7: The synthetic composition of any of Aspects 1-4, wherein said heterologous polynucleotide is an engineered guide RNA that is functional in a eukaryotic cell.

Aspect 8: The synthetic composition of any of Aspects 1-4, wherein said CRISPR-Cas effector protein further comprises a nuclear localization signal.

Aspect 9: The synthetic composition of any of Aspects 1-4, wherein said CRISPR-Cas effector protein is Cas-beta, or a functional fragment or functional variant thereof.

Aspect 10: The synthetic composition of any of Aspects 1-4, wherein said CRISPR-Cas effector protein is a fusion protein comprising a functional fragment or functional variant of Cas-beta.

Aspect 11: The synthetic composition of Aspect 10, wherein said fusion protein further comprises another nuclease domain.

Aspect 12: The synthetic composition of any of Aspects 1-4, further comprising at least one other protein.

Aspect 13: The synthetic composition of Aspect 12, wherein said other protein is selected from the group consisting of: Cas1, Cas2, and Cas4.

Aspect 14: The synthetic composition of Aspect 12, wherein said other protein is a complex comprising Cas1, Cas2, and one additional Cas protein, in that order.

Aspect 15: The synthetic composition of Aspect 14, wherein said additional Cas protein is Cas4.

Aspect 16: A synthetic composition of any of Aspects 1-14, wherein at least one component has been optimized for expression in a eukaryotic cell.

Aspect 17: The synthetic composition of Aspect 16, wherein said eukaryotic cell is from an organism selected from the group consisting of: maize, soybean, cotton, wheat, canola, oilseed rape, sorghum, rice, rye, barley, millet, oats, sugarcane, turfgrass, switchgrass, alfalfa, sunflower, peanut, potato, tobacco, Arabidopsis, safflower, and tomato.

Aspect 18: A synthetic composition comprising: (a) a polynucleotide encoding a CRISPR-Cas effector protein comprising the following: one positively charged bridge-helix-like domain, one bridge-helix domain, a DNA binding domain optionally comprising a helix-turn-helix motif, an RuvC-I domain, an RuvC-II domain, and an RuvC-III domain, and (b) a heterologous polynucleotide; wherein said effector protein does not comprise an HNH domain adjacent to the RuvC-II domain.

Aspect 19: The synthetic composition of Aspect 18, wherein said CRISPR-Cas effector protein comprises at least one domain upstream of the RuvC-I domain, selected from the group consisting of: a positively-charged bridge-helix like domain and a DNA binding domain optionally comprising a helix-turn-helix motif.

Aspect 20: The synthetic composition of Aspect 18, wherein said CRISPR-Cas effector protein comprises a plurality of domains in this order: a positively-charged bridge-helix-like domain, an RuvC-I domain, a bridge-helix domain, and RuvC-II domain, and an RuvC-III domain.

Aspect 21: A synthetic composition comprising: (a) a polynucleotide encoding a CRISPR-Cas effector protein derived from Armatimonadetes, and (b) a heterologous polynucleotide.

Aspect 22: The synthetic composition of any of Aspects 18-21, wherein said heterologous polynucleotide is

(a) derived from an organism of different taxonomy than that from which the effector protein was derived, (b) a different target than the natural target of the effector protein, or (c) completely or partially artificial.

Aspect 23: The synthetic composition of any of Aspects 18-21, wherein said heterologous polynucleotide is selected from the group consisting of: an expression element, a gene, a T-DNA, and a recombinant vector,

Aspect 24: The synthetic composition of any of Aspects 18-21, further comprising at least one gene encoding another protein.

Aspect 25: The synthetic composition of Aspect 24, wherein said gene encoding the another protein is selected from the group consisting of: cas1, cas2, and one additional cas gene.

Aspect 26: The synthetic composition of Aspect 25, wherein said additional cas gene is cas4.

Aspect 27: The synthetic composition of Aspect 24, wherein a plurality of said genes, each encoding a protein, are the following in this order: cas1, cas2, and one additional cas gene.

Aspect 28: The synthetic composition of Aspect 27, wherein said additional cas gene is cas4.

Aspect 29: A synthetic composition of any of Aspects 18-27, wherein at least one component has been optimized for expression in a eukaryotic cell.

Aspect 30: The synthetic composition of Aspect 29, wherein said eukaryotic cell is from an organism selected from the group consisting of: maize, soybean, cotton, wheat, canola, oilseed rape, sorghum, rice, rye, barley, millet, oats, sugarcane, turfgrass, switchgrass, alfalfa, sunflower, peanut, potato, tobacco, Arabidopsis, safflower, and tomato.

Aspect 31: A synthetic composition comprising an engineered, non-naturally occurring CRISPR-Cas system comprising: (a) a polynucleotide sequence encoding a CRISPR-Cas effector protein comprising at least two bridge-helix-like domains, a DNA binding domain optionally further comprising a helix-turn-helix motif, an RuvC-I domain, an RuvC-II domain, and an RuvC-III domain, and (b) a heterologous polynucleotide; wherein said effector protein does not comprise an HNH domain adjacent to the RuvC-II domain.

Aspect 32: A synthetic composition comprising an engineered, non-naturally occurring CRISPR-Cas system functional in eukaryotic cells, comprising: an effector protein derived from Armatimonadetes and a heterologous polynucleotide.

Aspect 33: The synthetic composition of Aspect 31, further comprising at least one gene encoding another protein.

Aspect 34: The synthetic composition of Aspect 33, further comprising a plurality of genes encoding at least two different other proteins.

Aspect 35: The synthetic composition of Aspect 33, wherein said gene encoding the another protein is selected from the group consisting of: cas1, cas2, and one additional cas gene.

Aspect 36: The synthetic composition of Aspect 35, wherein said additional cas gene is cas4.

Aspect 37: The synthetic composition of Aspect 34, wherein a plurality of said genes, each encoding a protein, are the following in this order: cas1, cas2, and one additional cas gene.

Aspect 38: The synthetic composition of Aspect 37, wherein said additional cas gene is cas4.

Aspect 39: The synthetic composition of Aspect 33, wherein said polynucleotide sequence encoding a CRISPR-Cas effector protein occurs upstream of at least one gene encoding another protein.

Aspect 40: The synthetic composition of Aspect 33, wherein said polynucleotide sequence encoding a CRISPR-Cas effector protein occurs upstream of cas1, cas2, and at least one additional cas gene.

Aspect 41: The synthetic composition of Aspect 39, wherein said additional cas gene is cas4.

Aspect 42: The synthetic composition of Aspect 31, wherein said heterologous polynucleotide comprises a regulatory element capable of effecting transcription of said polynucleotide sequence encoding a CRISPR-Cas effector protein.

Aspect 43: The synthetic composition of Aspect 31, wherein said polynucleotide sequence encoding a CRISPR-Cas effector protein has been sequence-optimized for improved expression in a eukaryotic cell.

Aspect 44: The synthetic composition of Aspect 43, wherein said eukaryotic cell is a plant cell or an animal cell.

Aspect 45: Any of the synthetic compositions of Aspects 1-41, wherein said gene or heterologous polynucleotide is comprised within a recombinant vector.

Aspect 46: Any of the synthetic compositions of Aspects 1-44, wherein said composition is attached to a solid matrix.

Aspect 47: A synthetic composition comprising: (a) a eukaryotic cell, (b) a heterologous CRISPR-Cas effector protein comprising at least the following: one positively charged bridge-helix-like domain, one bridge-helix domain, a DNA binding domain optionally comprising a helix-turn-helix motif, an RuvC-I domain, an RuvC-II domain, and an RuvC-III domain, and (c) a guide polynucleotide comprising a variable targeting domain that is substantially complementary to a target sequence in the genome of said cell; wherein said guide polynucleotide and heterologous effector protein are capable of forming a complex that can recognize, bind to, and optionally nick or cleave said target sequence; and wherein said effector protein does not comprise an HNH domain adjacent to the RuvC-II domain.

Aspect 48: The synthetic composition of Aspect 47, wherein said heterologous CRISPR-Cas effector protein comprises at least 500 amino acids.

Aspect 49: The synthetic composition of Aspect 47, wherein said heterologous CRISPR-Cas effector protein comprises less than 1000 amino acids.

Aspect 50: A synthetic composition comprising: (a) a eukaryotic cell, and (b) a heterologous CRISPR-Cas array comprising at least two repeats at least 90% identical to SEW NO: 11 wherein said repeats are separated by a spacer polynucleotide of at least 30 nucleotides.

Aspect 51: A synthetic composition comprising: (a) a eukaryotic cell, (b) a heterologous molecule selected from the group consisting of: a cas-beta gene capable of expressing a Cas-beta effector protein in said eukaryotic cell and a Cas-beta effector protein encoded by a cas-beta gene, and (c) a guide polynucleotide comprising a variable targeting domain that is substantially complementary to a target sequence in the genome of said cell; wherein said guide polynucleotide and Cas-beta effector protein are capable of forming a complex that can recognize, bind to, and optionally nick or cleave said target sequence.

Aspect 52: A synthetic composition comprising: (a) a eukaryotic cell, (b) at least two different heterologous molecules selected from the group consisting of: a cas1 gene capable of expression a Cas1 protein in said eukaryotic cell, a cas2 gene capable of expression a Cas2 protein in said eukaryotic cell, a cas4 gene capable of expression a Cas4 protein in said eukaryotic cell, a cas-beta gene capable of expression a Cas-beta effector protein in said eukaryotic cell, a Cas1 protein encoded by said cas1 gene, a Cas2 protein encoded by said cas2 gene, a Cas4 protein encoded by said cas4 gene, and a Cas-beta effector protein encoded by said cas-beta gene, and (c) a guide polynucleotide comprising a variable targeting domain that is substantially complementary to a target sequence in the genome of said cell; wherein said guide polynucleotide and cas-beta effector protein are capable of forming a complex that can recognize, bind to, and optionally nick or cleave said target sequence.

Aspect 53: The synthetic composition of Aspect 52, wherein said heterologous molecules of (b) is included within a continuous polynucleotide sequence comprising the following genes in this order: cas-beta, cas1, cas2, and one additional cas gene.

Aspect 54: The synthetic composition of Aspect 53, wherein said additional cas gene is cas4.

Aspect 55: The synthetic composition of Aspect 52, wherein said heterologous molecules of (b) is included within a continuous polypeptide sequence comprising the following proteins in this order: Cas-beta, Cas1, Cas2, and one additional Cas protein.

Aspect 56: The synthetic composition of Aspect 55, wherein said additional Cas protein is Cas4.

Aspect 57: The synthetic composition of any one of Aspects 47-56, wherein said eukaryotic cell further comprises a pre-existing heterologous polynucleotide.

Aspect 58: The synthetic composition of any of Aspects 1-57, wherein said effector protein, functional variant, or functional fragment thereof exhibits endonuclease activity.

Aspect 59: The synthetic composition of any of Aspects 1-57, further comprising a guide polynucleotide, wherein the guide polynucleotide comprises a sequence that shares substantial homology with the nucleotide sequence of a target site.

Aspect 60: The synthetic composition of any of Aspects 1-57, further comprising a guide polynucleotide, wherein the guide polynucleotide comprises at least two hairpin motifs, wherein one of the at least two hairpin motifs is GC-rich.

Aspect 61: The synthetic composition of any of Aspects 1-57, further comprising a guide polynucleotide, wherein the guide polynucleotide comprises at least two hairpin motifs, wherein one of the at least two hairpin motifs comprises the sequence UCCG.

Aspect 62: A method for modifying a target sequence in the genome of a eukaryotic cell, comprising: (a) introducing into said cell a composition from any one of Aspects 1-57 and a guide polynucleotide comprising a variable targeting domain that is substantially complementary to a target sequence in the genome of said cell, and (b) incubating said cell at a temperature greater than 10 degrees Celsius for a period of at least about four hours; wherein said guide polynucleotide and CRISPR-Cas effector protein are capable of forming a complex that can recognize, bind to, and optionally nick or cleave said target sequence.

Aspect 63: A method for modifying a target sequence in the genome of a eukaryotic cell, comprising: (a) introducing into said cell a Cas-beta effector protein or a cas-beta polynucleotide encoding said Cas-beta effector protein, and a guide polynucleotide comprising a variable targeting domain that is substantially complementary to a target sequence in the genome of said cell, and (b) incubating said cell at a temperature greater than 10 degrees Celsius for a period of at least about four hours; wherein said guide polynucleotide and Cas-beta endonuclease are capable of forming a complex that can recognize, bind to, and optionally nick or cleave said target sequence.

Aspect 64: The method of any one of Aspects 58-63, wherein said eukaryotic cell is a plant cell or an animal cell.

Aspect 65: The method of Aspect 64, wherein said plant cell is selected from the group consisting of: maize, soybean, cotton, wheat, canola, oilseed rape, sorghum, rice, rye, barley, millet, oats, sugarcane, turfgrass, switchgrass, alfalfa, sunflower, peanut, potato, tobacco, Arabidopsis, safflower, and tomato.

Aspect 66: The method of Aspect 64 or 65, wherein said cell further comprises a pre-existing heterologous polynucleotide.

Aspect 67: A polynucleotide modification template comprising at least one region that is complementary to a PAM sequence adjacent to a DNA target sequence recognized by a Cas-beta complex, wherein the at least one region that corresponds to a PAM sequence comprises at least one nucleotide mismatch.

Aspect 68: A method of producing a plant with a modified genome, comprising: (a) introducing into at least one cell of said plant a heterologous composition comprising: (a) a Cas-beta effector protein or a cas-beta polynucleotide encoding a Cas-beta effector protein,

(b) a guide polynucleotide comprising a variable targeting domain that is substantially complementary to a target sequence in the genome of said cell,

wherein said guide polynucleotide and Cas-beta endonuclease are capable of forming a complex that can recognize, bind to, and optionally nick or cleave said target sequence, (c) and a polynucleotide modification template comprising at least one region that is complementary to a PAM sequence adjacent to a DNA target sequence recognized by a Cas-beta complex, wherein the at least one region that corresponds to a PAM sequence comprises at least one nucleotide mismatch; (b) incubating said cell at a temperature greater than 10 degrees Celsius for a period of at least about four hours, (c) generating a whole plant from said cell, and (d) verifying at least one nucleotide modification in the genome of at least one cell of said plant as compared to the target sequence of the genome of said cell prior to the introduction of the heterologous composition of (a).

Aspect 69: The progeny of the plant obtained by the method of Aspect 68, wherein said progeny retains the at least one nucleotide modification of (d).

Aspect 70: A plant reproductive element from the plant obtained by the method of Aspect 33 or Aspect 69, wherein said plant reproductive element comprises at least one cell whose genome retains the at least one nucleotide modification of (d).

Aspect 71: A progeny plant derived from the plant reproductive element of

Aspect 70, wherein said progeny retains the at least one nucleotide modification of (d).

Aspect 72: A plurality of plant reproductive elements of the method of Aspect 70.

Aspect 73: A plurality of plants prepared according to the method of Aspect 68.

Aspect 74: The method of any one of Aspects 68-73, wherein said plant or plant reproductive element is selected from the group consisting of: maize, soybean, cotton, wheat, canola, oilseed rape, sorghum, rice, rye, barley, millet, oats, sugarcane, turfgrass, switchgrass, alfalfa, sunflower, peanut, potato, tobacco, Arabidopsis, safflower, and tomato.

Aspect 75: The method of any of Aspects 62-74, wherein said effector protein, functional variant, or functional fragment thereof exhibits endonuclease activity.

Aspect 76: A Cas endonuclease identified from a genomic locus comprising the following features in this order: (a) a gene encoding said Cas endonuclease, (b) a cas1 gene, (c) a cas2 gene, (d) a cas gene that is less than 90% identical to cas4, and (e) a CRISPR array comprising at least 35 repeat sequences, at least 30 of which share 100% sequence identity.

Aspect 77: A Cas endonuclease identified from a genomic locus comprising the following features in this order: (a) a gene encoding said Cas endonuclease, (b) a cas1 gene, (c) a cas2 gene, (d) a cas gene that is at least 70% identical to SEW NO:7, 8, or 9, and (e) a CRISPR array comprising at least 35 repeat sequences, at least 30 of which share 100% sequence identity.

Aspect 78: A Cas endonuclease identified from a genomic locus comprising the following features in this order: (a) a gene encoding said Cas endonuclease, (b) a cas1 gene, (c) a cas2 gene, (d) a cas gene that is less than 90% identical to cas4, and (e) a CRISPR array comprising at least 35 repeat sequences, at least 30 of which share at least 90% sequence identity to SEW NO:19.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific target site or target organism, the principles in these examples may be applied to any target site or target organism. Therefore, it will be appreciated that the scope of this invention is encompassed by the embodiments of the inventions recited herein and in the specification rather than the specific examples that are exemplified below. All cited patents, applications, and publications referred to in this application are herein incorporated by reference in their entirety, for all purposes, to the same extent as if each were individually and specifically incorporated by reference.

### EXAMPLES

The following are Examples of specific embodiments of some aspects of the invention. The Examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

### Example 1

### Identification and Characterization of Novel Class Cas-beta CRISPR-Cas Systems

In this Example, methods for identifying novel Class 2 CRISPR (Clustered Regularly Interspaced Short Palindromic Repeats)-Cas (CRISPR associated) loci using identification of operon-like gene architecture and protein structural analyses are described.

First, arrays of CRISPRs were detected within microbial sequences using PILER-CR (Edgar, R. (2007) BMC Bioinformatics, 8:18) and MinCED (Bland, C. et al. (2007) BMC Bioinformatics, 8:209) software programs. Next, known CRISPR-Cas systems were removed from the dataset by searching the proteins encoded in the vicinity (20 kb 5′ and 20 kb 3′ (where possible)) of the CRISPR array for homology with known CRISPR associated (Cas) proteins utilizing a set of position specific scoring matrices (PSSMs) encompassing all known Cas protein families as described in Makarova, K. et al. (2015) Nature Reviews Microbiology, 13:722-736. To aid in the complete removal of known Class 2 CRISPR-Cas systems, multiple-sequence alignment of protein sequences from a collection of orthologs from each family of Class 2 CRISPR-Cas endonucleases (e.g. Cas9, Cpf1 (Cas12a), C2c1 (Cas12b), C2c2 (Cas13), C2c3 (Cas12c)) was performed using MUSCLE (Edgar R. (2004) Nucleic Acids Res. 32:1792-1797). The alignments were examined, curated and used to build profile hidden Markov models (HMM) using HMMER (Eddy, S.R. (1998) Bioinformatics. 14:755-763; Eddy, S.R. (2011) PLoS Comp. Biol., 7:e1002195). The resulting HMM models were then utilized to further identify and remove known Class 2 CRISPR-Cas systems from the dataset. Next, using PSSM specific searches as described above, the CRISPR loci that remained were evaluated for the presence of genes encoding proteins implicated as being important for spacer insertion and adaptation, Cas1 and Cas2. CRISPR loci containing cas1 and cas2 genes were then selected and further examined to determine the proximity, order and directionality of the undefined genes encoded in the locus relative to the cas1 and cas2 genes and CRISPR array. Only those CRISPR loci forming an operon-like structure where a large (>1500 bp open-reading frame) undefined gene was present close to and in the same transcriptional direction as the cas1 and cas2 genes were selected for further analysis. Next, the protein encoded in the undefined gene was analyzed for sequence and structural features indicative of a Class 2 endonuclease that is capable of cleaving DNA. First, depending on how much similarity existed between a candidate sequence and known proteins, various bioinformatics tools were employed to reveal its conserved functional features, from pairwise comparison, to family profile search, to structural threading, and to manually structural inspection. In general, homologous sequences for a new candidate protein were first collected by a PSI-BLAST (Altschul, S. F. et al. (1997) Nucleic Acids Res. 25:3389-3402) search against the National Center for Biotechnology Information (NCBI) non-redundant (NR) protein collection with cut-off e-value of 0.01. After redundancy reduction at ˜90% identical level, groups of homologous sequences with various member inclusion thresholds (such as >60, 40, or 20% identity) were aligned to reveal conserved motifs by multiple-sequence alignment tools, MSAPRobs (Liu, Y. et. al. (2010) Bioinformatics. 26:1958-1964) and ClustalW. The most conserved homologous sequences underwent a sequence to family-profile search by HMMER (Eddy, S. R. (1998) Bioinformatics. 14: 755-763), against numerous domain databases including Pfam, Superfamily, and, SCOP (Murzin, A. G. et al. (1995) J. Mol. Biol. 247:536-540) and home-built structure-based profiles. Separately, the resulting candidate's homologous sequence alignment was also used to generate a candidate protein profile with addition of predicted secondary structures. The candidate profile was further used to do a profile-profile search by HHSEARCH (Soding, J. et al. (2006)Nucleic Acids Res. 34:W374-378), against pdb70_hhm and Pfam_hhm profile databases. In the next step, all detected sequence-structure relationships and the conserved motifs were threaded into a 3D structure template with MODELLER or manually mapped into the known structural reference on DiscoveryStudio (BIOVIA) and Pymol (Schrodinger). Finally, to verify and confirm the potential biological relevance as a Class 2 endonuclease, the catalytic or most conserved residues and key structural integrity were manually inspected and evaluated in light of the protein's biochemical function. Following structural identification of the key features indicative of a Class 2 endonuclease (e.g. DNA cleavage domain(s)), the other proteins encoded within the locus (5 kb 5′ and 5 kb 3′ from the ends of the newly defined CRISPR-Cas system (where possible)) were next examined for homology to known proteins families using InterProScan software (EMBL-EBI, UK) and through comparison with the NCBI NR protein collection using the BLAST program (Altschul, S. F. et al. (1990) J. Mol. Biol. 215:403-410). Genes encoding proteins with similarity (at least 30% identity) to known proteins were annotated as such in the CRISPR-Cas locus.

Initially, four novel Class 2 CRISPR-Cas system were identified from unknown microbes (Table 1). As shown in FIG. 1, encoded in each locus was an intact CRISPR-Cas system comprising all the components required for acquisition and interference. These included genes that together encoded proteins needed for acquiring and integrating spacers (Cas 1, Cas2, and optionally Cas4) and a novel protein comprising a DNA cleavage domain, Cas-beta (β), in an operon-like structure adjacent to a CRISPR array (Table 1A).

TABLE 1A
Novel Class 2 CRISPR-Cas Systems Cas-beta (β) 1, 2, 3 and 4

| | | Cas1 | Cas2 | Cas4 | Casβ SEQID NO | | Locus SEQID |
|---|---|---|---|---|---|---|---|
| Name | Organism | SEQID | SEQID | SEQID | Gene | Protein | NO |
| Cas β1 | Armatimonadetes | 1 | 4 | 7 | 10 | 13 | 16 |
| | bacterium | | | | | | |
| Cas β2 | Armatimonadetes | 2 | 5 | 8 | 11 | 14 | 17 |
| | bacterium | | | | | | |
| Cas β3 | Armatimonadetes | 3 | 6 | 9 | 12 | 15 | 18 |
| | bacterium | | | | | | |
| Cas β4 | Armatimonadetes | N/A* | 85 | 86 | 87 | 57 | 88 |
| | bacterium | | | | | | |

*ORF disrupted by gap in contig assembly

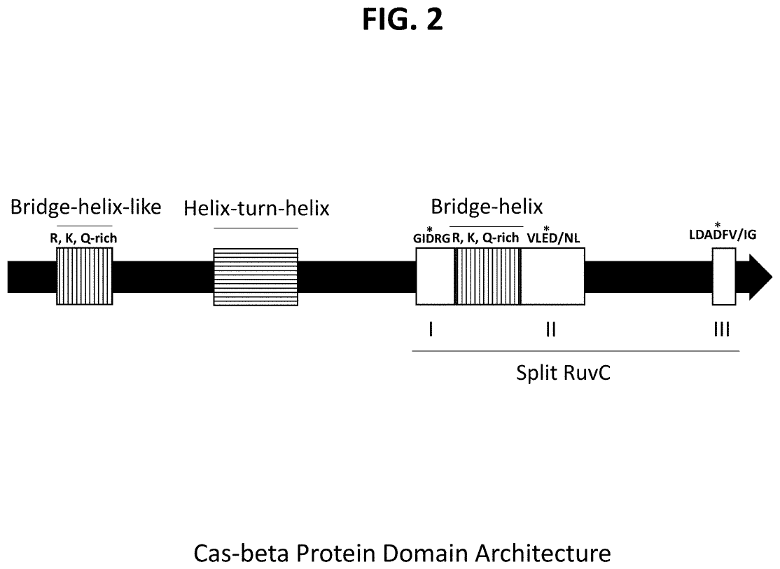

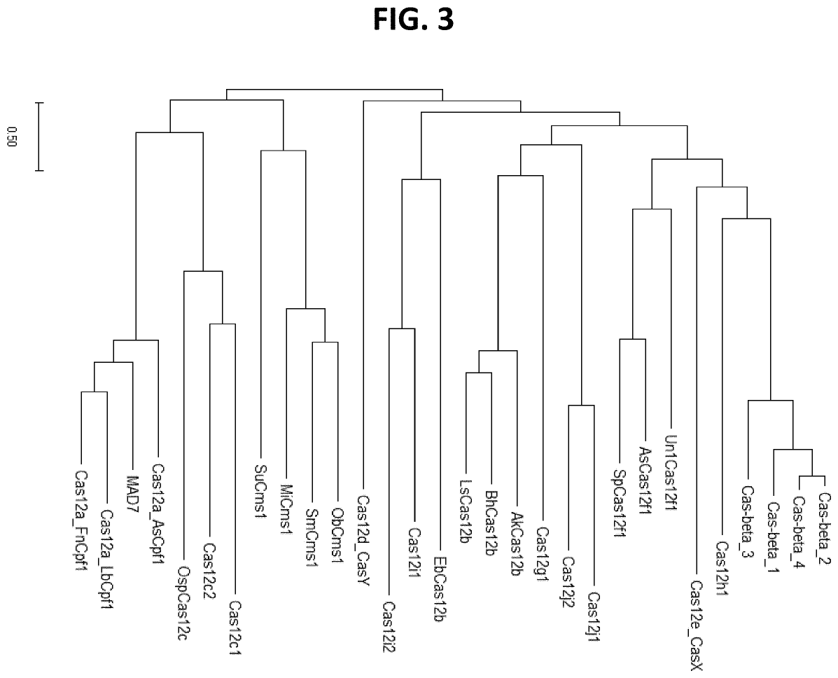

Like other type V CRISPR-Cas effector nucleases, structural examination revealed the presence of a tri-split RuvC-like nuclease domain located in the C-terminal half of the protein (FIG. 2). However, unlike previously characterized type V effectors, Cas-beta nucleases were observed to contain two bridge-helix (BH)-like motifs, one between RuvC subdomains I and II and the second near the N-terminal end of the protein (FIG. 2). A helix-turn-helix (HTH) motif with homology to a repeat condensin structure was also identified in the N-terminal half of the protein (FIG. 2). Furthermore, phylogenetic analyses supported their classification as being distinct from previously described type V nucleases (FIG. 3).

The positioning and orientation of the cas-beta nuclease and adaption genes within the CRISPR locus were also examined. In all, the gene locus architecture was unlike any class 2 type V CRISPR-Cas system previously reported and more closely resembled that of class 2 type II-B CRISPR-Cas systems (FIG. 1 and Makarova et al., 2019). Taken together, this further substantiates Cas-beta as a new class 2 CRISPR associated effector nuclease.

Cas-beta proteins share unique amino acid motifs, including those described in Table 1B.

TABLE 1B
Amino Acid Motifs of Cas-beta proteins
Conserved amino acids and motifs are described by the start position
numbers in each protein (x = any or no amino acid, in any number).

| | Cas-beta | | | |
|---|---|---|---|---|
| Amino Acid/Motif | 1 | 2 | 3 | 4 |
| LR | 17 | 8 | 16 | 18 |
| RL(Q/E)RL | 24 | 15 | 23 | 25 |
| KIxxH | 34 | 25 | 33 | 35 |
| LH(F/Y)LLH(K/Q)(I/V)EVE(R/Y) | 51 | 43 | 50 | 53 |
| D(L/I)xRNL | 64 | 56 | 63 | 66 |
| PKP | 80 | 68 | 76 | 78 |
| KxxxRxE | 84 | 72 | 79 | 82 |
| PxxP | 99 | 86 | 93 | 96 |
| I | 117 | 104 | 113 | 114 |
| PxP | 120 | 107 | 116 | 117 |
| GRxW | 146 | 133 | 137 | 143 |
| EV | 156 | 143 | 147 | 153 |
| LP | 159 | 146 | 150 | 156 |
| LxxxxxxD(D/E) | 171 | 158 | 162 | 168 |
| VRxWA | 183 | 170 | 174 | 180 |
| SIE | 193 | 180 | 184 | 190 |
| AR(E/D) | 197 | 184 | 188 | 194 |
| Lx(D/E) | 227 | 217 | 226 | 227 |
| D | 235 | 234 | 234 | 244 |
| R | 240 | 239 | 239 | 249 |
| QxxWV | 247 | 246 | 246 | 256 |
| GVxKDTxRR | 265 | 264 | 264 | 274 |
| L(K/R)XFHxxxxNS | 279 | 278 | 278 | 288 |
| KHTNPQFPYL | 290 | 289 | 303 | 299 |
| VV | 311 | 310 | 323 | 320 |
| R | 326 | 325 | 339 | 335 |
| RxxxLQxELGxA | 339 | 338 | 352 | 341 |
| R | 355 | 354 | 370 | 357 |
| WxGxxxxxGT | 363 | 362 | 378 | 365 |
| (R/K)(R/K)R | 375 | 374 | 390 | 377 |
| LVWD | 381 | 380 | 396 | 383 |
| KID | 403 | 402 | 418 | 405 |
| YxDG | 411 | 410 | 426 | 413 |
| LxSDXQ | 417 | 416 | 432 | 419 |
| CxxxPLxxKHxFLRW(Y/F) | 434 | 433 | 449 | 436 |
| (K/R)HxxNH | 451 | 450 | 466 | 453 |
| APLE(R/K)RCxHXTTQ | 460 | 459 | 475 | 462 |
| FVxxD | 473 | 472 | 490 | 475 |
| PRLF(I/V)RPVFKFYD | 483 | 482 | 501 | 485 |
| KPxCRYL(I/V)GIDRG(I/V)NYxxR | 509 | 508 | 527 | 511 |
| DxE | 532 | 531 | 550 | 534 |
| KxxW | 548 | 547 | 566 | 550 |
| RxE(I/L)AY(F/H)Q | 555 | 554 | 573 | 557 |
| R(R/K)(R/K)(D/E)RxxLx(K/R) | 585 | 588 | 607 | 591 |
| TV | 596 | 599 | 618 | 602 |
| GxxNYCFVLEXL | 612 | 615 | 634 | 618 |
| NLxRNNRVKH | 629 | 632 | 650 | 635 |
| EAL(I/V)xQMRKxGY | 644 | 647 | 665 | 650 |
| (R/K)(V/A)DGVRxxE | 663 | 666 | 683 | 669 |
| YTSxxxPxGWWAK(R/K)(D/E)EV(E/D) | 674 | 677 | 694 | 680 |
| KxD | 695 | 698 | 715 | 701 |
| RK(I/V)GxxY | 704 | 707 | 724 | 710 |
| GRx(K/R) | 742 | 745 | 748 | 748 |
| Px(n)RR | 755 | 758 | 760 | 761 |
| GSELFWDP | 764 | 767 | 770 | 770 |
| GxxF | 778 | 781 | 784 | 784 |
| GVVLDADF(V/I)GAxNIALRPLV | 784 | 787 | 790 | 790 |
| G(K/R) | 808 | 811 | 814 | 814 |
| HxxxNP | 820 | 823 | 826 | 826 |
| C | 831 | 834 | 837 | 837 |
| YEF | 836 | 839 | 842 | 842 |
| LRRI | 849 | 852 | 855 | 855 |

### Example 2

### Cas-Beta Guide RNA Solutions

In this Example, methods for determining the guide RNA(s) that support double stranded DNA target recognition and cleavage for a novel group of class 2 CRISPR (Clustered Regularly Interspaced Short Palindromic Repeat)-Cas (CRISPR associated) endonucleases, Cas-beta, are described.

One method relies on computational prediction to determine the small RNA(s) needed to form a functional complex with a Cas-beta endonuclease. Briefly, the CRISPR array may be utilized to generate CRISPR RNA(s) (crRNA(s)) accounting for both possible transcriptional directions of the CRISPR array and various configurations of the repeat and spacer (e.g. repeat:spacer, spacer:repeat or repeat:spacer:repeat) that may be preferred by the endonuclease. Additionally, a trans-encoding CRISPR associated RNA(s) (tracrRNA(s)) may be computationally identified in the locus as described in Karvelis, T. et al. (2015) Genome Biology. 16:253. Briefly, an alignment may be performed between the CRISPR repeat consensus sequence with the locus sequence using BLAST or manually by hand. Regions of homology (separate from the CRISPR array) may then be examined by analyzing the possible transcriptional directions of the putative tracrRNA(s) for secondary structures and possible termination signals present in an RNA version of the sense and anti-sense genomic DNA sequences surrounding the anti-repeat. Additionally, the regions encoding the putative tracrRNA may be aligned (using MUSCLE or equivalent) to identify conserved sequence motifs and features. The tracrRNA(s) may then be duplexed with various crRNA predictions or engineered to form a chimeric non-natural single guide RNA(s) (sgRNA(s)). crRNA(s), tracrRNA(s) and sgRNA(s) may be synthesized (IDT equivalent) or T7 transcribed with the TranscriptAid T7 High Yield Transcription Kit (Thermo Fisher Scientific) or equivalent for further experimentation.

Another method is reliant on the sequencing of small RNAs (sRNA-seq) that are produced from the novel Class 2 CRISPR-Cas locus. This may be performed similar to the method described in Zetsche, B. et al. (2015) Cell. 163:1-13. Briefly, the CRISPR-Cas locus is placed onto an E. coli plasmid DNA, subsequent cultures containing the plasmid borne CRISPR-Cas locus are harvested by centrifugation, total RNA extracted using TRIzol Max Bacterial Isolation Kit (Thermo Fisher Scientific), small RNAs isolated using the mirVana miRNA Isolation Kit (Thermo Fisher Scientific) and libraries prepared for sequencing using the TruSeq Small RNA Library Prep Kit (Illumina). Expression of the locus can be boosted using known E. coli promoters. Following sequencing on a MiSeq instrument (Illumina) or equivalent, the resulting sequence data is mapped (Bowtie 2 software (Langmead, B. et al. (2012) Nat. Methods. 9:357-359) or equivalent) back to the locus to determine the transcriptional and maturation patterns of the sRNA(s) encoded in the locus.

Another method is reliant on the sequencing of small RNAs (sRNA-seq) that are co-purified with the Cas-beta protein from the novel Class 2 CRISPR-Cas locus. This may be performed similar to the method described in Sinkunas, T. et al. (2013) EMBO J. 32:385-394 except Illumina deep sequencing may be employed to determine the sequence of the small RNA(s) needed to direct double stranded DNA target recognition and cleavage. Briefly, the CRISPR-Cas locus is placed onto an E. coli plasmid DNA. The cas-beta gene in the locus can be modified to also encode a protein purification tag. For example but not limited to a histidine (His), StrepTag II (Strep), and/or maltose binding protein (MBP) tag. Alternatively, a “solo” Cas-beta expression cassette encoding a His, Strep, and/or MBP tagged version of the Cas-beta protein can be co-transformed with the plasmid borne locus. Next, the plasmid(s) are transformed into E. coli (for example but not limited to Artic Express (DE3) (Agilent Technologies) and then cultures are harvested by centrifugation. The cells are then lysed and tagged Cas-beta protein purified by chromatography. Finally, small RNAs bound by the Cas-beta protein are extracted using TRIzol Max Bacterial Isolation Kit (Thermo Fisher Scientific) or other suitable method (e.g NEBNext® Small RNA Library Prep, NEBNext® rRNA Depletion Kit (Bacteria), and Monarch® RNA Cleanup Kits) and processed as described above.

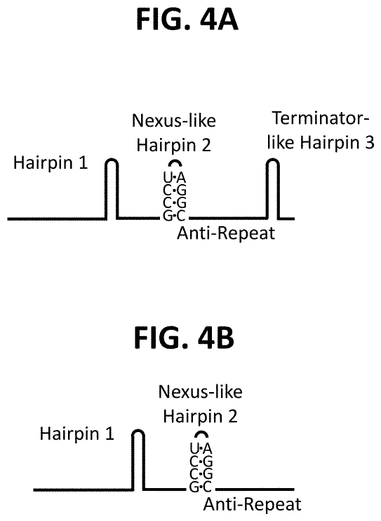

The crRNA, tracrRNA, and sgRNA solutions for the Cas-beta systems described herein are listed in Table 2. Conserved structural features of Cas-beta tracrRNAs are shown in FIGS. 4A and B.

TABLE 2
Cas-beta (β) guide RNA solutions

| | Repeat | | | |
|---|---|---|---|---|
| | Consensus | crRNA | tracrRNA | sgRNA |
| | (SEQID | (SEQID | (SEQID | (SEQID |
| Name | NO.) | NO.) | NO.) | NO.) |
| Casβ1 | 19 | 22 | 25, 26 | 31, 32 |
| Casβ2 | 20 | 23 | 27, 28 | 33, 34 |
| Casβ3 | 21 | 24 | 29, 30 | 35, 36 |
| Casβ4 | 20 | 23 | 89, 90 | 91, 92 |

### Example 3

### Bacterial Cas-Beta Expression Plasmids

In this Example, plasmid DNA expression constructs are generated to examine Cas-beta double stranded DNA target recognition and cleavage in a heterologous host, E. coli.

First, the CRISPR array of a native Cas-beta CRISPR-Cas locus (FIG. 1) encoding the first Cas-beta endonuclease examined herein, Cas-beta 2 (SEQID NO:14), was modified. This was accomplished by reducing the number of CRISPR units (repeat (SEQID NO:20):spacer:repeat (SEQID NO:20)) to between two and four. Next, the spacer sequence between the repeats was replaced with a sequence capable of base pairing with the anti-sense strand of a double stranded target sequence adjacent to a 7 bp region of randomization from the plasmid DNA PAM library described in Karvelis et al., 2015, T2 (SEQID NO:37). The resulting “complete” CRISPR-Cas locus engineered to target T2, was then synthesized (GenScript) directly into a low copy E. coli plasmid DNA modified to contain a single isopropyl β-D-1-thiogalactopyranoside (IPTG) inducible T7 promoter (pETduet-1, MilliporeSigma) resulting in plasmid DNA Cas-beta2_pETduet-1.

Additionally, a “solo” cas-beta gene fused to a sequence encoding a histidine (HIS) tag (6X-HIS SEQID NO:38 or 10X-HIS SEQID NO:39, or 14X-HIS SEQID NO:58), maltose binding protein (MBP) tag (SEQID NO:40), small ubiquitin-like modifier (SUMO) tag (SEQID NO:41) and optionally a simian virus 40 (SV40) nuclear localization signal (NLS) (SEQID NO:71) was constructed by methods known in the art (for example but not limited to Golden Gate or Gibson assembly (Potapov et al. 2018 and Gibson et al. 2010)). Native cas-beta gene sequences or E. coli codon optimized versions were utilized. For optimized genes, codon conditioning was carried out using E. coli codon tables, genes adjusted for ideal GC content, and repetitive sequences and gene destabilizing features removed where possible. Finally, the tagged “solo” cas-beta genes were cloned into either a Tetracycline (TET), IPTG, or arabinose inducible plasmid DNA expression cassettes by standard techniques (for example but not limited to Golden Gate or Gibson assembly (Potapov et al. 2018 and Gibson et al. 2010)).

### Example 4

### Cas-Beta Protein Expression and Purification

In this Example, a method to recombinantly express and purify Cas-beta endonucleases are described.

Cas-beta protein was expressed and purified using a tagged “solo” protein expression plasmid as described in Example 3. First, the expression construct was transformed into either E. coli BL21(DE3) or Nico (DE3) strains and cultures were grown in LB broth supplemented with selective agent (e.g. kanamycin (50 μg/ml)). After culturing to an OD600 of 0.5, temperature was decreased to 16° C. and expression induced with IPTG (0.4 mM)). After 16 h, cells were pelleted and re-suspended in loading buffer (20 mM Tris-HCl, pH 8.0 at 25° C., 0.3 M NaCl and 40 mM imidazole) and disrupted by sonication. Cell debris was removed by centrifugation. The supernatant was loaded on a DEAE Sepharose FF column (GE healtcare). The flow through was collected and then loaded on a Ni²⁺-charged HiTrap chelating HP column (GE Healthcare) and eluted with a linear gradient of increasing imidazole concentration (from 40 to 500 mM) in 20 mM Tris-HCl, pH 8.0 at 25° C. and 0.3 M NaCl. The fractions containing Cas-beta were pooled and the tag was cleaved by overnight incubation with SenP1 protease at 4° C. in the presence of 1 mM DTT. To remove cleaved His-MBP-SUMO tag and SenP1 protease, reaction mixtures were loaded onto a HiTrap heparin HP column (GE Healthcare) or size exclusion column for elution using a linear gradient of increasing NaCl concentration (from 0.3 to 1.5 M). Next, the elution from the HiTrap columns was loaded on a MBPTrap column (GE Healthcare) and Cas-beta protein was collected as flow though. The collected fractions were then dialyzed against 20 mM Tris-HCl, pH 8.0 at 25° C., 500 mM NaCl, 1 mM DTT, and 50% (v/v) glycerol and stored at -20° C.

### Example 5

### Methods to Detect Cas-Beta Double Stranded DNA Target Recognition and Cleavage

In this Example, methods to detect double strand DNA target recognition and cleavage by Cas-beta endonucleases are described.

### Lysate Assay

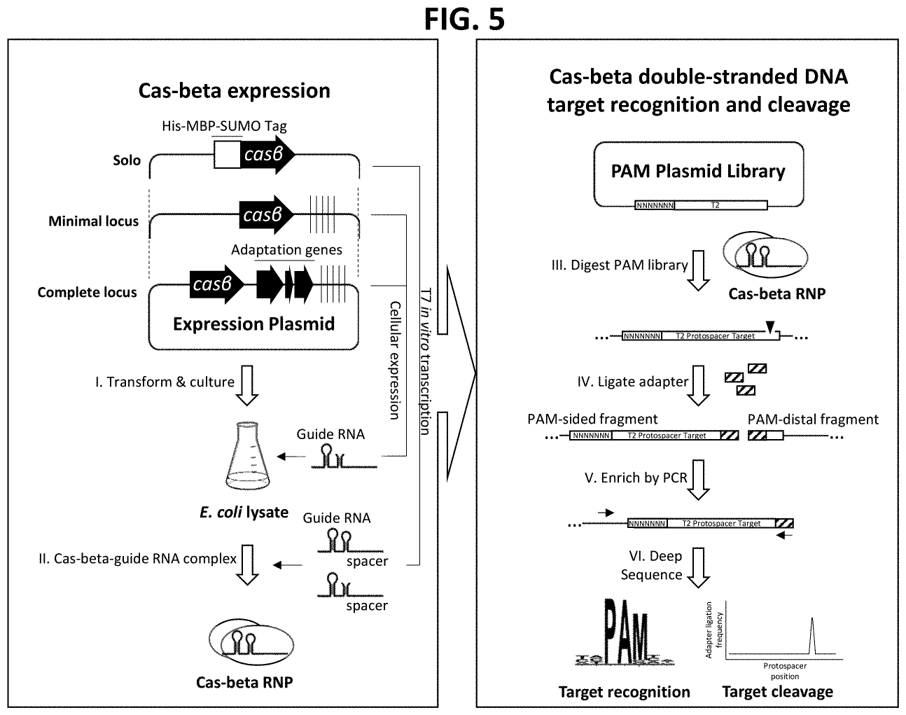

The detection of double stranded DNA target recognition and cleavage was carried-out using cell lysates expressing a Cas-beta endonuclease as shown in FIG. 5. First, a plasmid DNA encoding a Cas-beta endonuclease either by itself or as part of a Cas-beta CRISPR-Cas locus modified to target T2 (see Example 3) was transformed into E. coli cells (e.g., BL21 (DE3) or DH5α (Thermo Fisher Scientific), ArcticExpress (DE3) (Agilent Technologies) or Nico (DE3)) by methods known in the art. Next, cell cultures carrying the gene encoding the Cas-beta endonuclease were cultured to an optical density (OD) of 0.5 (using a wavelength of 600 nm) in Luria broth (LB) media containing a suitable antibiotic (e.g., ampicillin) (FIG. 5 Step I). For plasmids that required an inducing agent to stimulate expression (e.g., Cas-beta2_pETduet-1), the temperature was decreased to 16° C., and expression initiated with inducing agent (e.g., 0.4 mM isopropyl β-D-1-thiogalactopyranoside (IPTG)) for 16 h. If no induction was required, cells were immediately harvested after reaching an OD600 of 0.5. Next, cells were pelleted by centrifugation (at 3,000 g for 5 min at 4° C.), media poured-off, and resuspended in 1 ml of lysis buffer (20 mM phosphate, pH 7.0, 0.5 M NaCl, 5% (v/v) glycerol) supplemented with 10 μl PMSF and transferred to ice. The cells were then disrupted by sonication for 2 min (6 s pulse followed by 3 s pause) and cell debris removed by centrifugation at 14,000 g for 30 min at 4° C. Next, for Cas-beta proteins expressed as a solo component, 20 μl of supernatant containing the soluble Cas-beta protein was immediately combined with 2 μg of the T7 transcribed guide RNA(s) in the presence of 1 μl (40 U) of RiboLock RNase Inhibitor (Thermo Fisher Scientific) and incubated for 15 min at room temperature (FIG. 5 Step II). If the Cas-beta endonuclease and guide RNAs were expressed together from the plasmid borne CRISPR-Cas locus, the clarified lysate containing Cas-beta guide RNA ribonucleoprotein complexes was not processed any further but used directly in the next step (FIG. 5 Step II). Digestion of a randomized PAM library was then performed by gently combining 10 μl of the Cas-beta guide RNA lysate mixture with 90 μl of reaction buffer (10 mM Tris-HCl, pH 7.5 at 37° C., 100 mM NaCl and 1 mM DTT, 10 mM MgCl2) and 1 μg of the 7 bp randomized PAM library from Karvelis et al. 2015 (FIG. 5 Step III). Alternatively, if the PAM sequence was known, 10 μl of the Cas-beta guide RNA lysate mixture was combined with 1 μg of plasmid DNA containing a fixed target sequence. After 1 h at 37° C., reactions were subject to DNA end-repaired by incubating them with 1 μl (5U) of T4 DNA polymerase and 1 μl of 10 mM dNTP mix (Thermo Fisher Scientific) for 20 min at 11° C. The reaction was then inactivated by heating it to 75° C. for 10 min. To efficiently capture free DNA ends by adapter ligation, a 3′-dA overhang was added by incubating the reaction mixture with 1 μl (5 U) of DreamTaq polymerase (Thermo Fisher Scientific, EP0701) for 30 min at 72° C. Excess RNA was then removed from the reaction by incubating 1 μl of RNase A/T1 (Thermo Fisher Scientific) for 30 min at 37° C. The resulting DNA was then purified using a GeneJet PCR Purification Kit (Thermo Fisher Scientific).

Next, an adapter with a 3′-dT overhang was prepared by annealing Al (5′-CGGCATTCCTGCTGAACCGCTCTTCCGATCT-3′ (SEQID NO:42)) and phosphorylated A2 (5′-GATCGGAAGAGCGGTTCAGCAGGAATGCCG-3′ (SEQID NO:43)) oligonucleotides by heating an equimolar mixture of the two for 5 min at 95° C. and slowly cooling (−0.1° C/s) to room temperature in Annealing (A) buffer (10 mM Tris-HCl, pH 7.5 at 37° C., 50 mM NaC1). The adapter was then ligated to the end repaired 3′-dA overhanging cleavage products by combining 100 ng of it and the adapter with 5 U of T4 Ligase (Thermo Fisher Scientific) in 25 μl of ligation buffer (40 mM Tris-HCl, pH 7.8 at 25° C., 10 mM MgC12, 10 mM DTT, 0.5 mM ATP, 5% (w/v) PEG 4000) and allowing the reaction to proceed for 1 h at room temperature (FIG. 5 Step IV).

Next, the cleaved products containing the PAM sequence were enriched using RO (for T2 cleavage (5′-GCCAGGGTTTTCCCAGTCACGA-3′ (SEQID NO:44))) and the Al oligonucleotide specific to the 7 bp PAM library and adapter, respectively (FIG. 5 Step V). PCR was performed with Phusion High-Fidelity PCR Master Mix in HF buffer (New England Biolabs) using 10 μl of the ligation reaction as template. A two-step amplification protocol (98° C.-30 s initial denaturation, 98° C.-15 s, 72° C.-30 s denaturation, annealing and synthesis for 15 cycles and 72° C.-5 min for final extension) was used. For the samples assembled in the absence of a Cas-beta, PCR was performed using RO and the CO primer (5′-GAAATTCTAAACGCTAAAGAGGAAGAGG-3′ (SEQID NO:45)) pair with CO being complementary to protospacer sequence. Next, the amplification products were purified using a GeneJet PCR Purification Kit (Thermo Fisher Scientific).

Next, the sequences and indexes required for Illumina deep sequencing were incorporated onto the ends of the Cas-beta cleaved DNA fragments and the resulting products deep sequenced (FIG. 5 Step VI). This was accomplished through two rounds of PCR using Phusion High-Fidelity PCR Master Mix in HF buffer (New England Biolabs) per the manufacturer's instruction. The primary PCR was assembled using 20 ng of Cas-beta cleaved adapter ligated PAM-sided template and allowed to proceed for 10 cycles. The reaction uses a forward primer, F1 (5′ -CTACACTCTTTCCCTACACGACGCTCTTCCGATCTAAGGCGGC-ATTCCTGCTGAAC-3′ (SEQID NO:46)) that can hybridize to the adapter and a reverse primer, R1 (5′ -CAAGCAGAAGACGGCATACGAGCTCTTCCGATCTCGGCGACGTTGGGTC-3′ (SEQID NO:47)), that binds to a site 3′ of the region of PAM randomization. In addition to hybridizing to the adapter ligated PAM fragment, the primers also contain Illumina sequences extending off their 5′ ends. For the forward primer, the extra sequence includes a portion of the sequence required for bridge amplification (5′-CTACACTCTTTCCCTACACGACGC-TCTTCCGATCT-3′ (SEQID NO:48)) followed by an interchangeable unique index sequence (5′-AAGG-3′) that permits multiple amplicons to be deconvoluted if sequenced simultaneously. For the reverse primer, the additional sequence is comprised only of that required for bridge amplification at the 3′ end of the amplicon (5′ -CAAGCAGAAGACGGCATACGAGCTC-TTCCGATCT-3′ (SEQID NO:49)). The following PCR cycling conditions were used:95° C.-30 s initial denaturation, 95° C.-10 s, 60° C.-15 s, 72° C.-5 s denaturation, annealing and synthesis for 10 cycles and 72° C.-5 min for final extension. Following primary PCR, a second round of PCR amplification was performed using 2 μl (in total volume of 50 μl) of the first round PCR as template. The forward primer, F2 (5′-AATGATACGGCGACCACCGAGATCTACACTCTTT-CCCTACACG-3′ (SEQID NO:50)), used in the secondary PCR hybridizes to the 5′ region of F1 further extending the sequences required for Illumina deep sequencing. The reverse primer, R2 (5′-CAAGCAGAAGACGGCATA-3′ (SEQID NO:51)), used in the secondary PCR simply binds to the 3′ end of the primary PCR amplicon. The following PCR cycling conditions were used:95° C.-30 s initial denaturation, 95° C.-10 s, 58° C.-15 s, 72° C.-5 s denaturation, annealing and synthesis for 10 cycles and 72° C.-5 min for final extension. Following library creation, amplifications were purified with a QlAquick PCR Purification Kit (Qiagen) per the manufacturer's instruction and combined into a single sample in an equimolar concentration. Next, the libraries were single-read deep sequenced on a MiSeq Personal Sequencer (Illumina) with a 25% (v/v) spike of PhiX control v3 (Illumina) and sequences post-processed and deconvoluted per the manufacture's instruction. Note the original PAM library was also sequenced as a control to account for inherent bias that would affect downstream PAM analyses. This is carried out as described above except the forward primer in the primary PCR, Cl (5′-CTACACTCTTTCCCTACACGACGCTCTTCCGATCTGGAATAAACGCTAAAGAGGAAG AGG-3′ (SEQID NO:52)), is used instead of F1 as it hybridizes directly to the protospacer region in the uncut PAM library.

Next, evidence of double stranded DNA target recognition was evaluated by searching for the presence of a PAM in the Cas-beta cleaved fragments. This was accomplished by first generating a collection of sequences representing all possible outcomes of double stranded DNA cleavage and adapter ligation within the target region. For example, cleavage and adapter ligation at just after the 23r^{d} position of the target would produce the following sequence (5′- GCTCTTCCGATCTACGCCGGCGACGTTGGGTCAACT-3′ (SEQID NO:53)) where the adapter and target sequences comprise 5′-GCTCTTCCGATCT-3′ (SEQID NO:54) and 5′-ACGCCGGCGACGTTGGGTCAACT-3′ (SEQID NO:55), respectively. Next, these sequences were searched for in the sequence datasets along with a 10 bp sequence 5′ of the 7 bp PAM region (5′-TGTCCTCTTC-3′ (SEQID NO:56)). Once identified, the intervening PAM sequence was isolated by trimming away the 5′ and 3′ flanking sequences. Next, the frequency of the extracted PAM sequences was normalized to the original PAM library to account for bias inherent to the initial library. First, identical PAM sequences were enumerated, and frequency calculated versus the total reads in the dataset. Then, normalization was performed for each PAM using the following equation such that PAM sequences that were under- or over-represented in the initially library were accounted for:

Normalized Frequency=(Treatment Frequency)/(((Control Frequency)/(Average Control Frequency)))

After normalization, a position frequency matrix (PFM) was calculated. This was done by weighting each nucleotide at each position based on the frequency (normalized) associated with each PAM. For example, if a PAM of 5′-CGGTAGC-3′ had a normalized frequency of 0.15%, then the C at first position would be given a frequency of 0.15% when determining the nucleotide frequency for the first PAM position. Next, the overall contribution of each nucleotide at each position in the dataset was summed and organized into a table with the most abundant nucleotides indicating Cas-beta PAM preferences.

Evidence for Cas-beta double stranded DNA target cleavage was evaluated by examining the unique junction generated by Cas-beta target cleavage and adapter ligation. First, a collection of sequences representing all possible outcomes of double stranded DNA cleavage and adapter ligation within the T2 target region were generated (as detailed above). Next, the frequency of the resulting sequences was examined in each Illumina sequence dataset relative to negative controls (experiments setup without Cas-beta). Protospacer-adapter ligation positions where Illumina sequences were recovered in excess resulting in a peak or spike of read coverage over negative controls were considered as evidence of targeted DNA cleavage.

### Example 6

### Cas-Beta Double Stranded DNA Target Recognition and Cleavage

In this Example, the molecular features that impart Cas-beta double stranded DNA target recognition and cleavage are identified.

Cas-Beta is a PAM-Dependent dsDNA Endonuclease

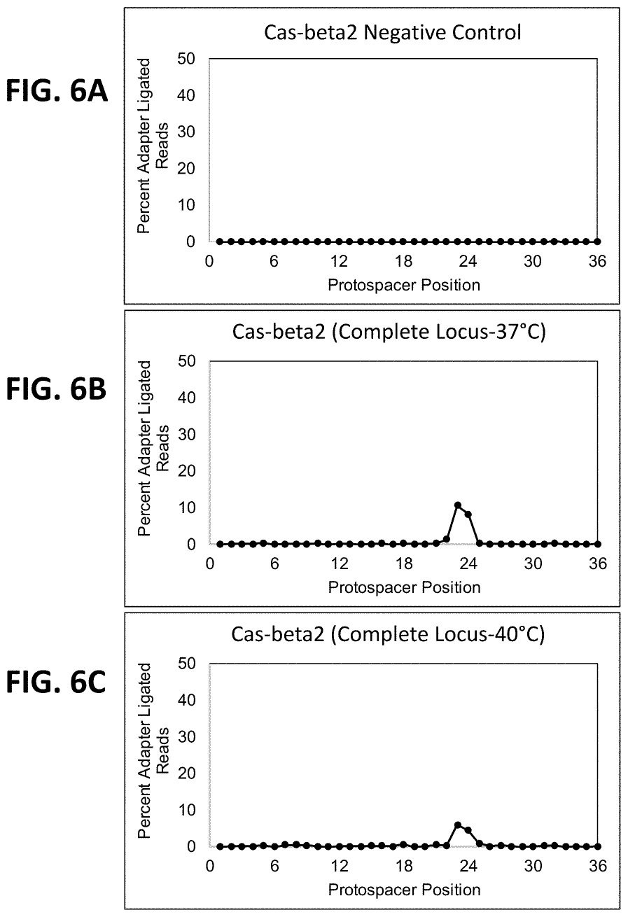

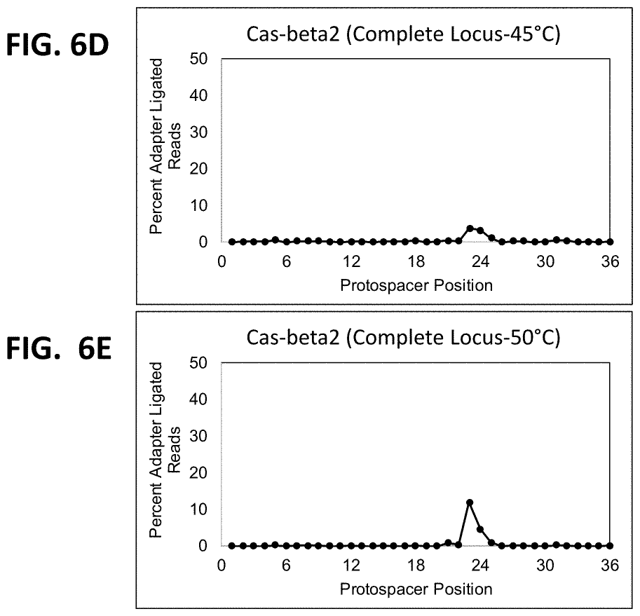

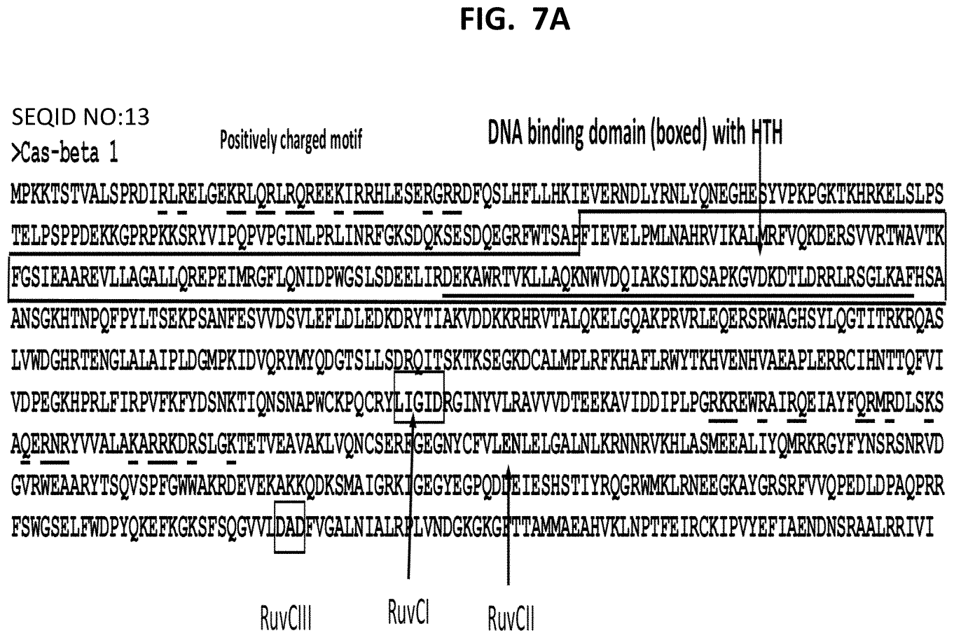

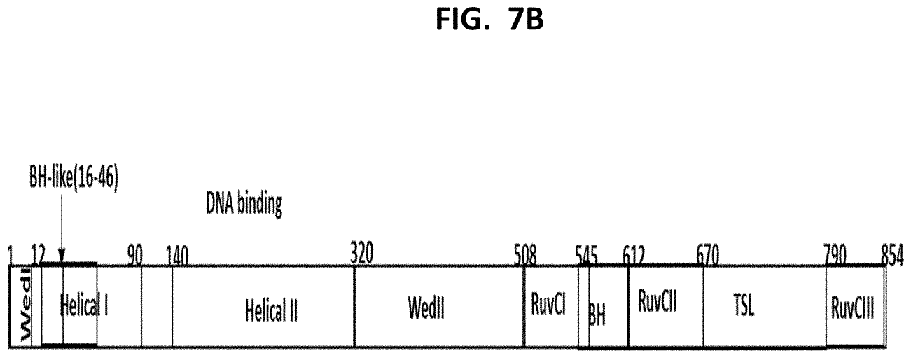

As shown in FIGS. 6A-E, a spike in the recovery of adapter ligated sequence reads relative to negative control experiments was recovered in the T2 target sequence just after the 23r^{d} and 24^{th} positions 3′ of the PAM region (depending on the temperature of the experiment) when using plasmid Cas-beta2pETduet-1 (containing a fully intact Cas-beta CRISPR-Cas locus modified to target the T2 sequence). Moreover, as shown in Table 3, fragments with adapters ligated at these positions exhibited preferences for a C-rich motif in the randomized PAM library, altogether, providing the first evidence of Cas-beta double stranded DNA target recognition and cleavage.

TABLE 3
Protospacer adjacent motif (PAM) preferences for Cas-beta2
Displayed as a position frequency matrix (PFM). PAM positions are numbered backward from
the first position of the protospacer target with position −1 being immediately 5’ of the first
position of the T2 protospacer target. Numbers in brackets [x] represent strong PAM preferences,
numbers in slashes /x/ represent weak PAM preferences.

| | | PAM Position | | | | | | |
|---|---|---|---|---|---|---|---|---|
| | | −7 | −6 | −5 | −4 | −3 | −2 | −1 |
| Nucleotide | T | 24.63% | 32.16% | 28.56% | 23.03% | 0.19% | 0.14% | [41.52%] |
| | G | 17.82% | 23.95% | 23.93% | 24.20% | 0.06% | 0.06% | 14.70% |
| | C | 18.02% | 25.13% | 21.87% | 14.92% | [99.37%] | [99.74%] | [36.37%] |
| | A | 39.53% | 18.76% | 25.64% | 37.84% | 0.38% | 0.05% | 7.41% |
| Consensus | | N | N | N | N | C | C | Y |

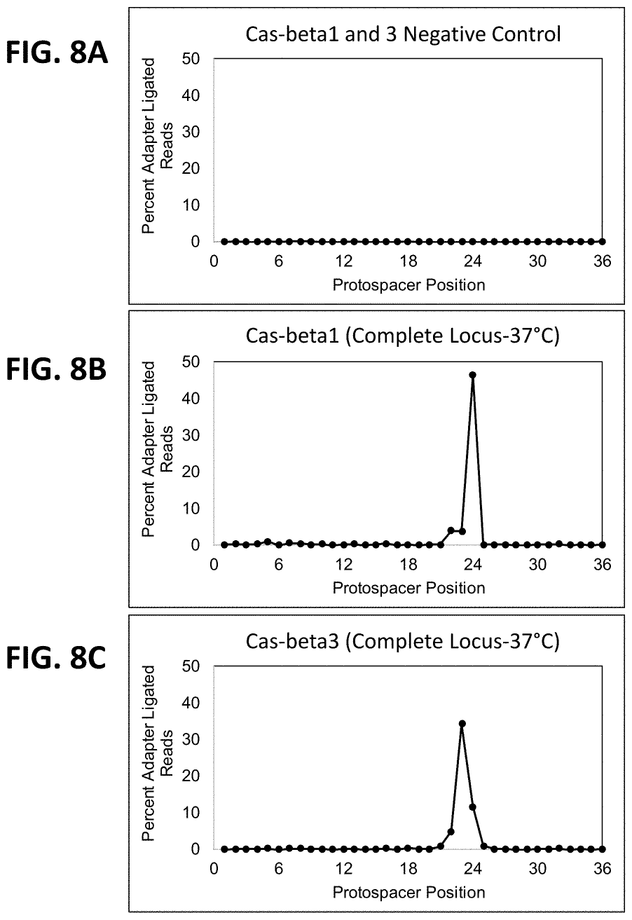

Fully intact Cas-betal and 3 CRISPR-Cas loci modified to target the T2 sequence also yielded an enrichment in the recovery of adapter ligated reads in the T2 target (FIGS. 8A-C). In this case, the majority or reads indicated that the position of target cleavage (following T4 DNA polymerase end-repair) was just after either the 24^{th} or 23r^{d} positions 3′ of the region of PAM randomization for Cas-betal and 3, respectively. Similar to Cas-beta2, the library fragments that supported Cas-betal and 3 target cleavage showed that it also preferred a 5′ C-rich PAM (Tables 4 and 5).

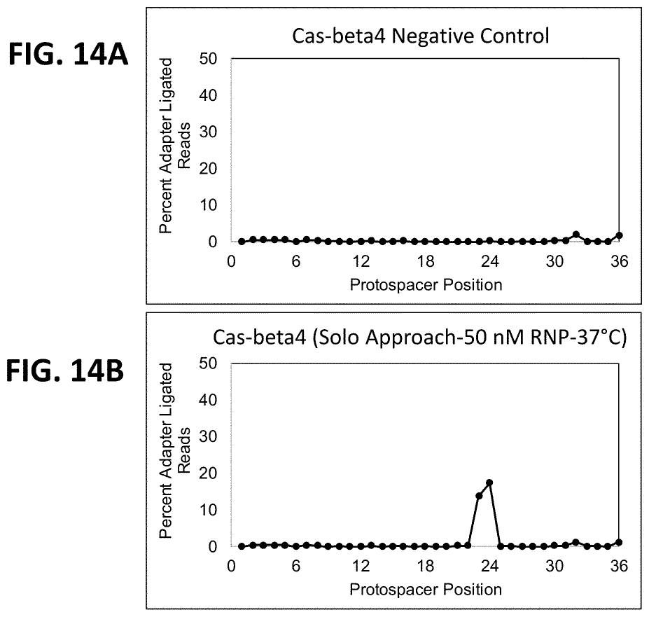

The locus encoding Cas-beta4 was disrupted by a gap in DNA sequence contig assembly, therefore, its ability to recognize and cleave double stranded DNA was assessed using purified Cas-beta4 and in vitro transcribed sgRNA designed to target the 7N PAM plasmid library (FIG. 5 “solo” approach). As shown in FIGS. 14A and B, Cas-beta4 also recognized and cleaved a double stranded DNA target. Like Cas-beta2, cleavage was detected just after the 23^{rd} and 24^{th} base pair 3′ of the PAM region (FIGS. 6B and 14B). Moreover, Cas-beta4 like Cas-betal, 2 and 3 recognized a C-rich PAM (Tables 3-6).

TABLE 4
Protospacer adjacent motif (PAM) preferences for Cas-beta1
Displayed as a position frequency matrix (PFM). PAM positions are numbered backward from
the first position of the protospacer target with position −1 being immediately 5’ of the first
position of the T2 protospacer target. Numbers in brackets [x] represent strong PAM preferences,
numbers in slashes /x/ represent weak PAM preferences.

| | | PAM Position | | | | | | |
|---|---|---|---|---|---|---|---|---|
| | | −7 | −6 | −5 | −4 | −3 | −2 | −1 |
| Nucleotide | T | 27.75% | 26.48% | 25.84% | 25.36% | 0.57% | 0.47% | [37.82%] |
| | G | 23.58% | 25.23% | 22.75% | 24.65% | 0.53% | 0.51% | 4.18% |
| | C | 22.64% | 25.78% | 22.39% | 16.51% | [98.16%] | [98.32%] | [54.31%] |
| | A | 26.04% | 22.51% | 29.03% | 33.47% | 0.73% | 0.70% | 3.69% |
| Consensus | | N | N | N | N | c | c | Y |

TABLE 5
Protospacer adjacent motif (PAM) preferences for Cas-beta3
Displayed as a position frequency matrix (PFM). PAM positions are numbered backward from
the first position of the protospacer target with position −1 being immediately 5’ of the first
position of the T2 protospacer target. Numbers in brackets [x] represent strong PAM preferences,
numbers in slashes /x/ represent weak PAM preferences.

| | | PAM Position | | | | | | |
|---|---|---|---|---|---|---|---|---|
| | | −7 | −6 | −5 | −4 | −3 | −2 | −1 |
| Nucleotide | T | 30.36% | 29.99% | 30.00% | 22.94% | 0.23% | 0.15% | [42.56%] |
| | G | 22.60% | 19.44% | 21.29% | 25.77% | 0.13% | 0.12% | 12.07% |
| | C | 23.89% | 27.99% | 23.85% | 12.50% | [99.37%] | [99.53%] | [41.58%] |
| | A | 23.15% | 22.57% | 24.86% | 38.79% | 0.27% | 0.20% | 3.79% |
| Consensus | | N | N | N | N | C | C | Y |

TABLE 6
Protospacer adjacent motif (PAM) preferences for Cas-beta4
Displayed as a position frequency matrix (PFM). PAM positions are numbered backward from
the first position of the protospacer target with position −1 being immediately 5’ of the first
position of the T2 protospacer target. Numbers in brackets [x] represent strong PAM preferences,
numbers in slashes /x/ represent weak PAM preferences.

| | | PAM Position | | | | | | |
|---|---|---|---|---|---|---|---|---|
| | | −7 | −6 | −5 | −4 | −3 | −2 | −1 |
| Nucleotide | T | 25.62% | 26.54% | 26.62% | 27.35% | 1.04% | 4.24% | [31.97%] |
| | G | 26.24% | 25.38% | 24.11% | 26.39% | 0.11% | 0.20% | 2.85% |
| | C | 23.73% | 25.37% | 25.31% | 18.70% | [96.69%] | [95.46%] | [60.34%] |
| | A | 24.41% | 22.71% | 23.97% | 27.57% | 2.16% | 0.10% | 4.85% |
| Consensus | | N | N | N | N | C | C | Y |

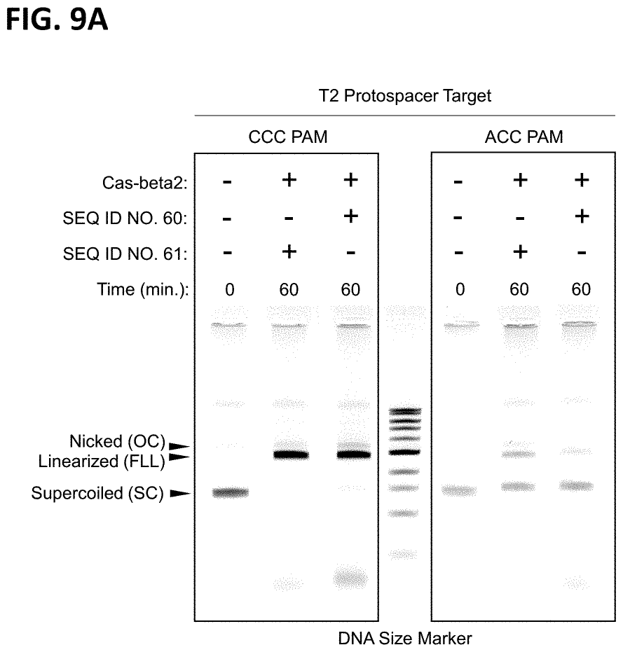

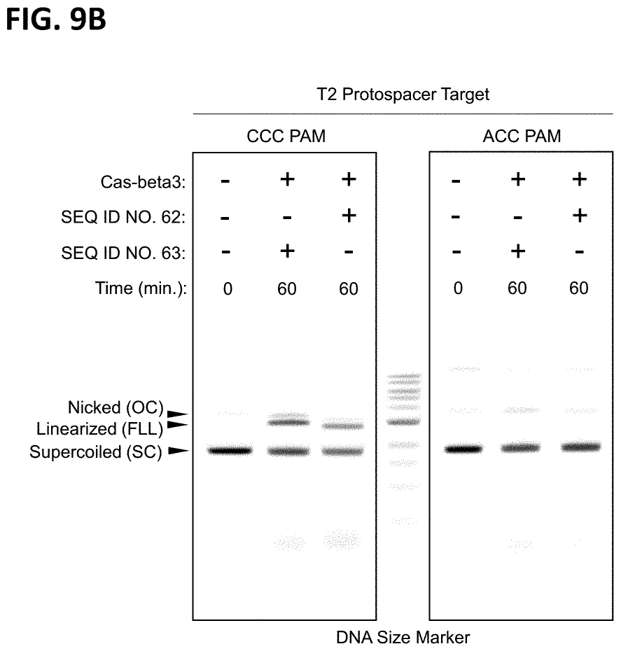

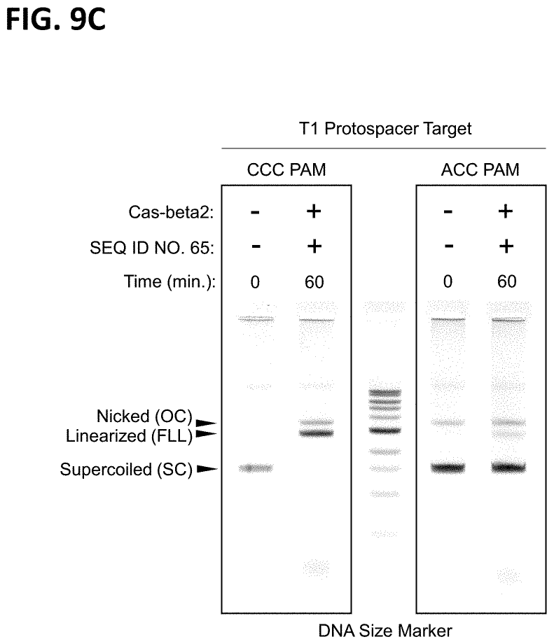

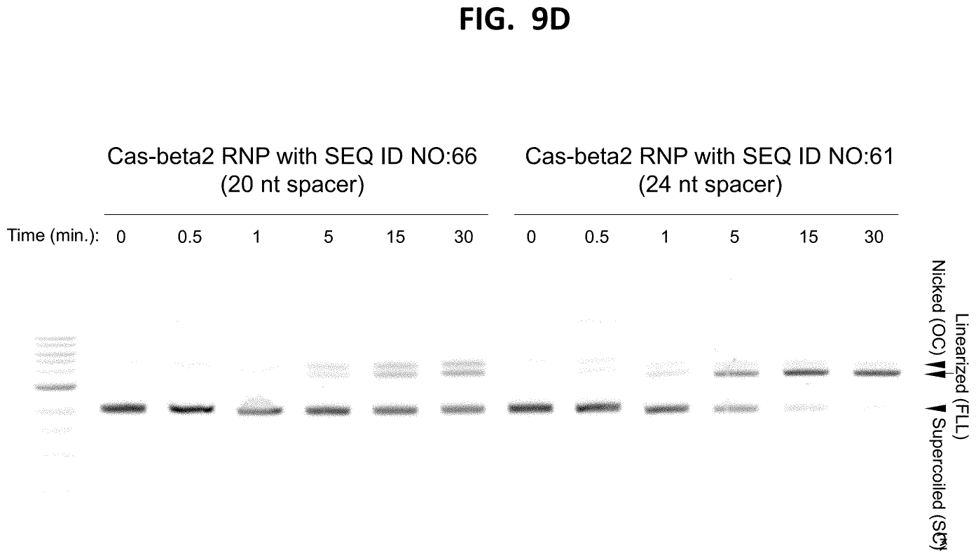

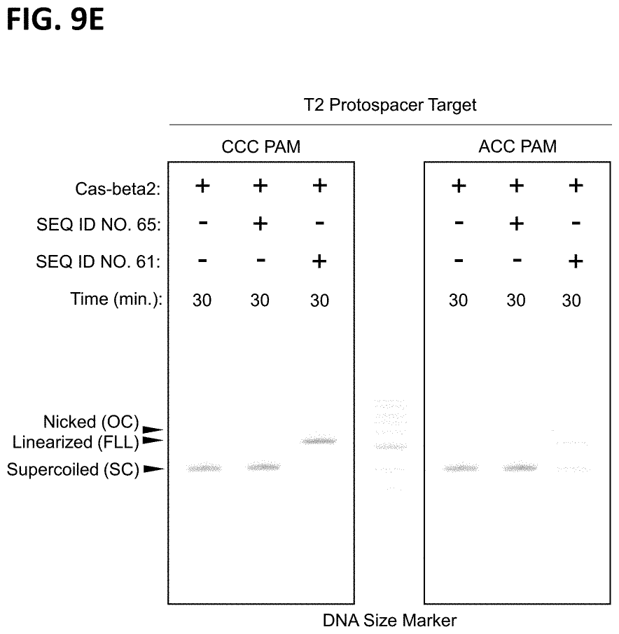

Next, we reconstituted Cas-beta2 and 3 double stranded DNA target cleavage activity using purified components in vitro. First, DNA fragments encoding a T7 polymerase promoter, transcription initiation signal (5′-G′-3) and the sgRNAs described in Example 2 (SEQID NOs:33-36) linked to a 24 nt spacer sequence (SEQID NO:59) with complementarity to the T2 target sequence were generated similar to that described previously (Karvelis et al. (2020) Nucleic Acids Res. 48:5016-5023). Next, sgRNAs (SEQID NOs:60-63) were in vitro transcribed using a TranscriptAid T7 High Yield Transcription Kit (Thermo Fisher Scientific). Ribonucleoprotein (RNP) complexes were then assembled by combining purified Cas-beta protein (Example 4) with the respective sgRNA in a 1:2 molar ratio in complex assembly buffer (10 mM Tris-HCl, pH 7.5 at 37° C., 100 mM NaCl, 1 mM EDTA, 1 mM DTT) and incubating the mixture for 15 min. at room temperature. 50 nM of the resulting RNP was then combined with 5 nM of supercoiled (SC) plasmid DNA containing a preferred (5′-CCC-3′) or unpreferable (5′-ACC-3′) Cas-beta PAM immediately 5′ of the sgRNA target in reaction buffer (CutSmart, NEB). After a 60 min. incubation at 37° C., reactions were quenched with phenol/chloroform, aqueous phase mixed with 3x loading dye (0.01% (w/v) bromophenol blue and 75 mM EDTA in 50% (v/v) glycerol, and analyzed by agarose gel electrophoresis and ethidium bromide staining. As described earlier (Jinek, M. et al. (2012) Science. 337:816-821) and Gasiunas et al. (2012) Proc. Natl. Acad. Sci. U.S.A. 109:E2579-2586), SC plasmid DNA substrate allows both target nicking resulting in relaxed open circle (OC) plasmid DNA and full length linear (FLL) double stranded DNA target cleavage to be monitored. As shown in FIGS. 9A and B, while some OC product was observed, most Cas-beta activity resulted in the conversion of SC plasmid DNA to a single FLL form, thus, demonstrating the formation of a target double strand DNA break. Additionally, the 5′-CCC-3′ PAM supported more robust DNA target cleavage activity than did the 5′-ACC-3′ PAM for both Cas-beta2 and 3 RNP complexes (FIGS. 9A and 9B) corroborating the 5′ PAM preferences determined for Cas-beta2 and 3 (Tables 3 and 4).

To check that the Cas-beta endonucleases could be reprogrammed to cleave a different target site, new SC plasmid substrates were first generated containing a different target sequence, T1 (SEQID NO:64), adjacent to both 5′-CCC-3′ and 5′-ACC-3′ PAMs. Next, a DNA template encoding a T7 promoter, transcription initiation signal (5′-G′-3) and sgRNA from Cas-beta2 (SEQID NO:34) linked to the T1 sequence was constructed as described above. Using this fragment as template, a Ti targeting sgRNA (SEQID NO:65) was then generated by T7 in vitro transcription. When RNP complexes comprised of Cas-beta2 protein and the T1 targeting sgRNA were combined with the new SC plasmid templates in vitro, a single FLL product was also observed (FIG. 9C). Similar to the T2 target, the fraction of substrate fully cleaved was also higher for the 5′-CCC-3′ PAM (FIG. 9C).

To examine the effect of guide RNA spacer length on Cas-beta2 endonuclease efficiency, two sgRNAs were generated. The first contained a 20 nt spacer targeting T2 (SEQID NO:66) and the second had a 24 nt spacer (SEQID NO:61). Next, RNP digests were performed using the SC plasmid substrate containing the 5′-CCC-3′ PAM immediately 5′ of the T2 target as described above except reactions were stopped at 0.5, 1, 5, 15, and 30 min. As shown in FIG. 9D, sgRNAs with both spacer lengths converted the plasmid DNA into a linearized form with the sgRNA containing the 24 nt spacer supporting more rapid DNA target cleavage.

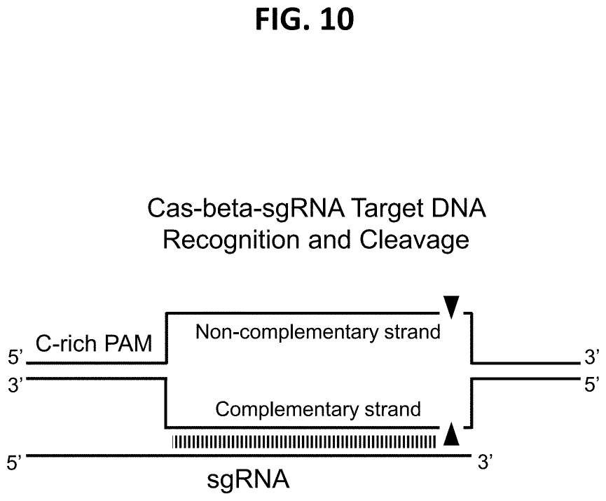

To further confirm that the observed double stranded DNA target cleavage by Cas-beta endonucleases was RNA-guided, additional experiments were assembled. This included a Cas-beta protein only control as well as reactions where the spacer of the sgRNA didn't match the SC plasmid DNA target. As shown in FIG. 9E, the only digest that yielded FLL product came from those reactions where the sgRNA spacer had homology with the protospacer target. Altogether, this shows that Cas-beta protein and its sgRNA are the sole components required for recognizing and cleaving a double stranded DNA target (with said target site comprising a C-rich PAM upstream (5′) of a region with complementarity to its guide RNA (FIG. 10)).

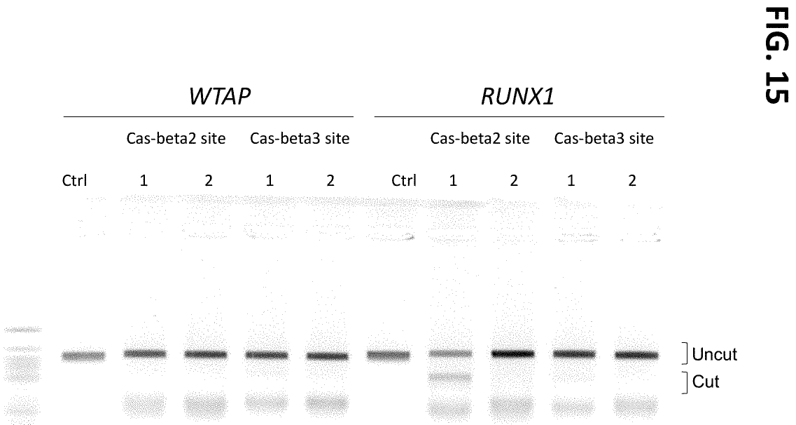

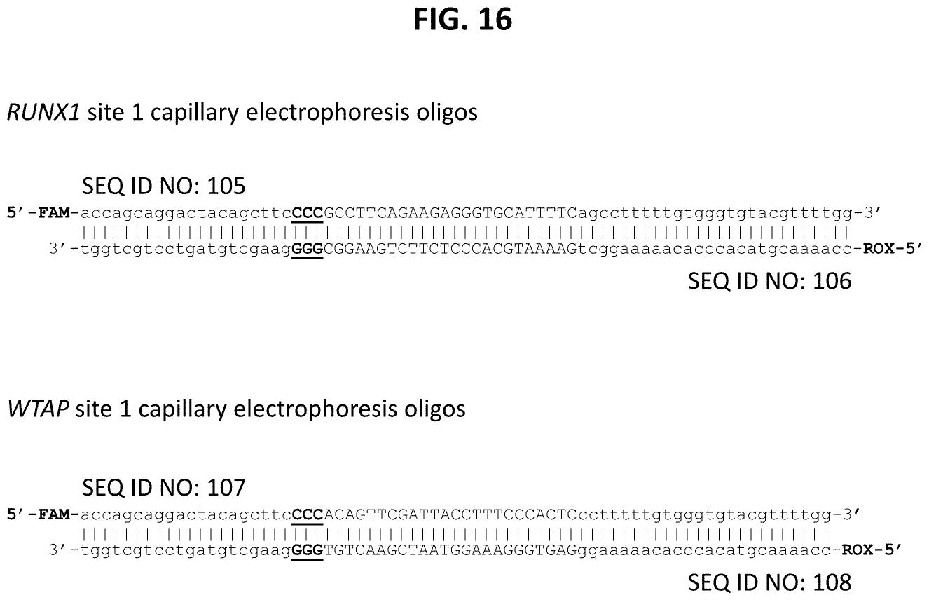

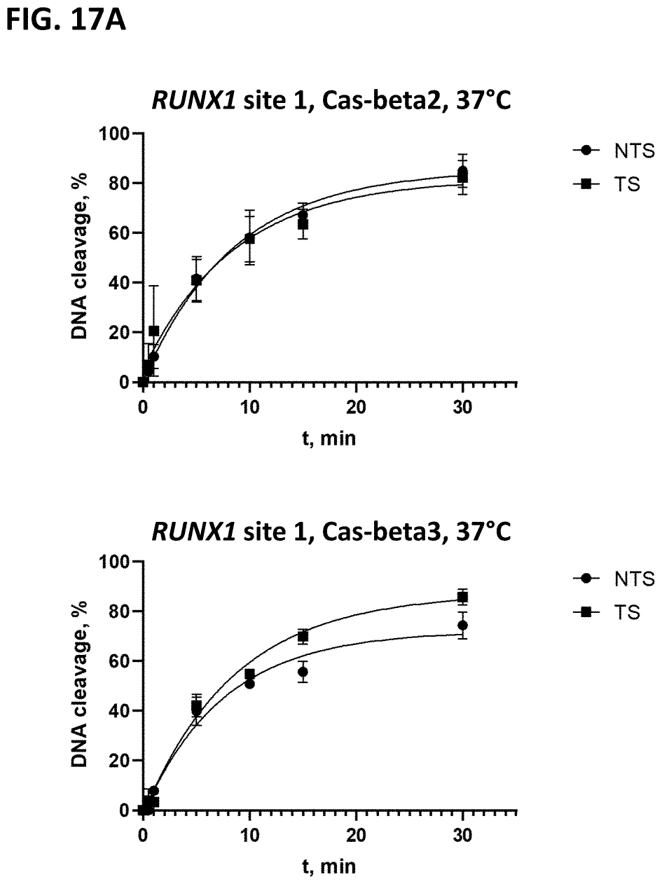

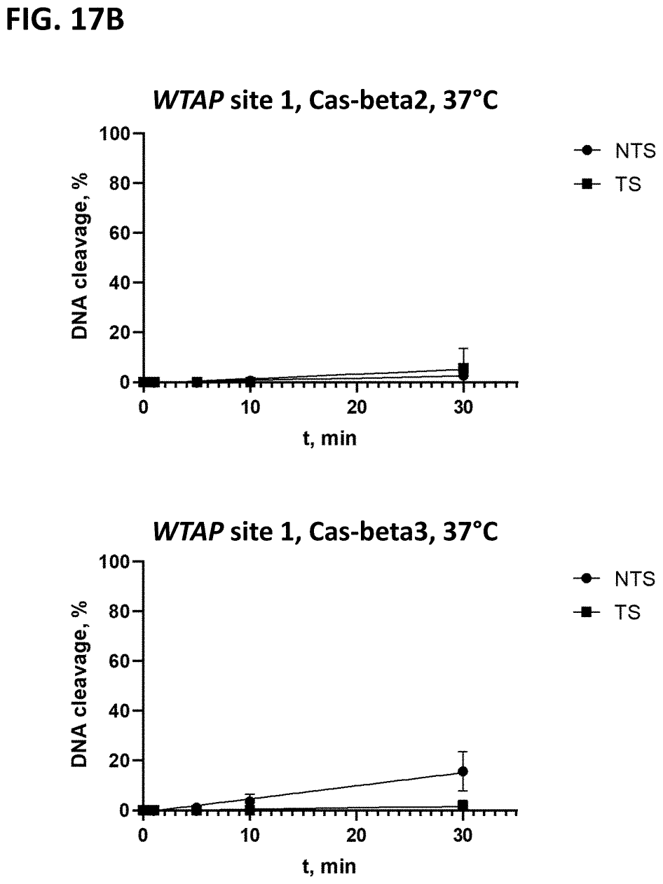

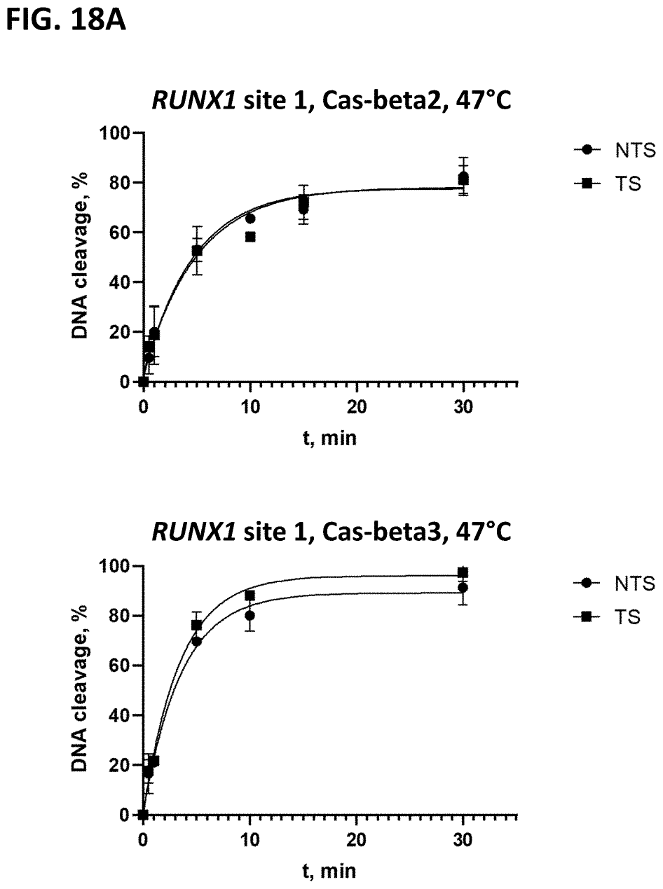

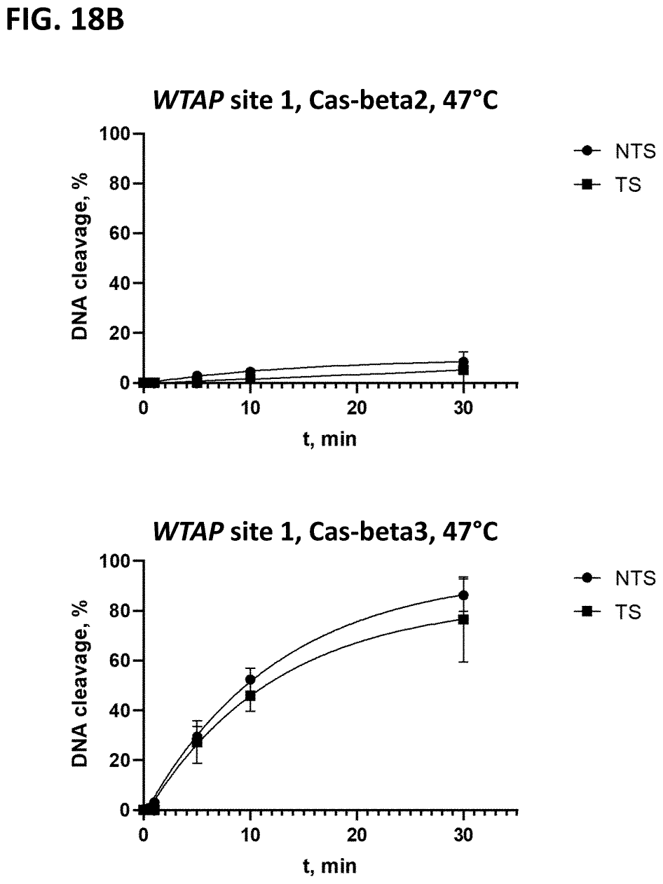

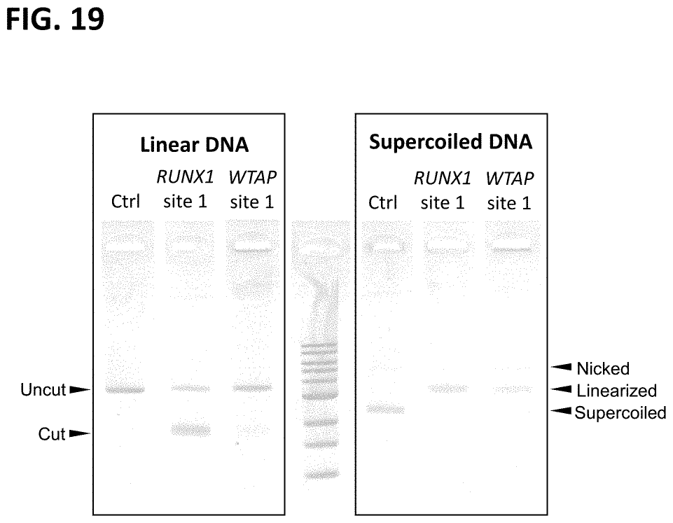

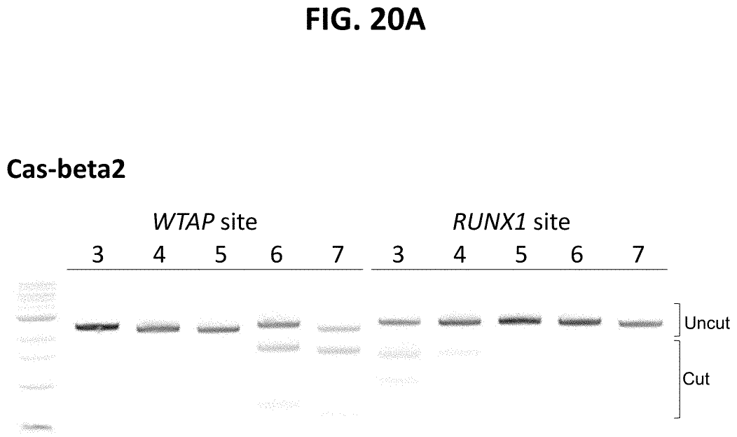

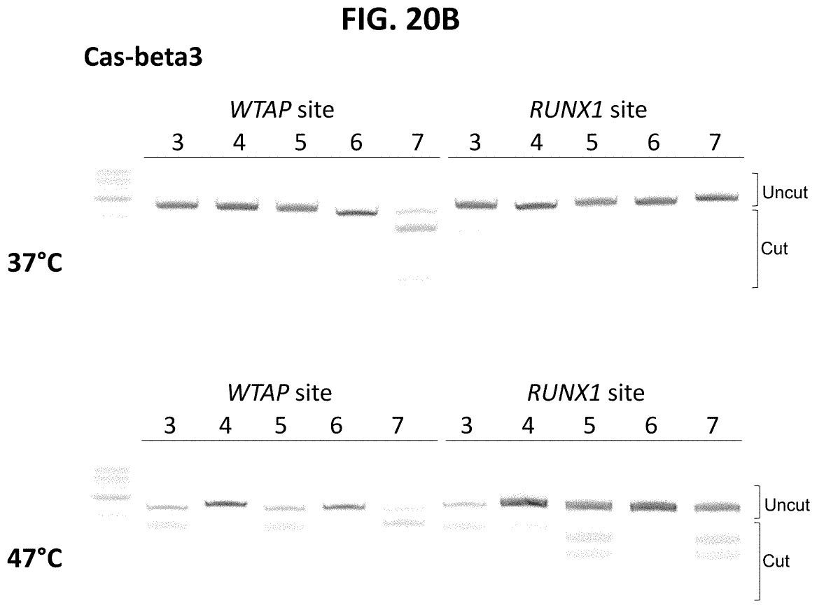

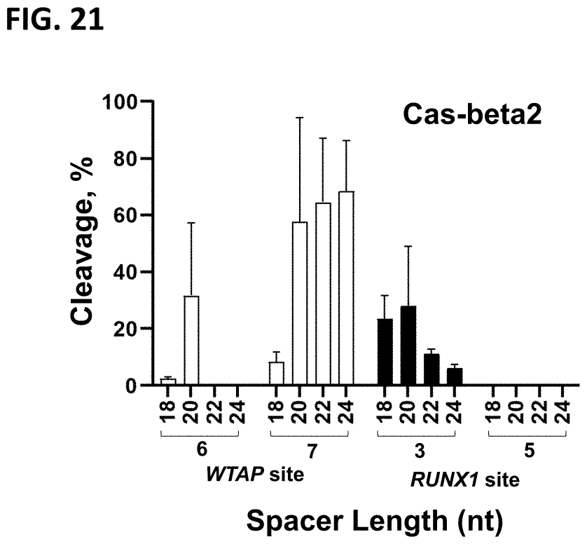

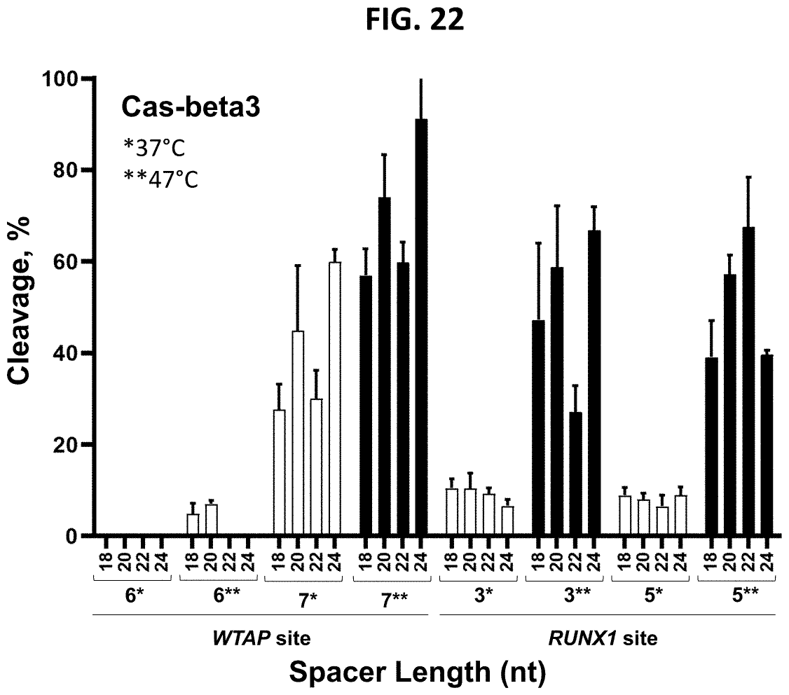

The biochemical parameters conducive for Cas-beta double stranded DNA target recognition and cleavage were next explored. Initially, four additional targets were selected in the WTAP and RUNX1 genes (two in each gene) (SEQID NO: 93-96) and sgRNAs capable of guiding Cas-beta2 and 3 to these sites were generated as described earlier (SEQID NOs: 97-104). The genomic regions containing each target site were then PCR amplified for use as substrate in digestion reactions assembled with Cas-beta2 and 3. Here, 50 nM of Cas-beta RNP was combined with 5 nM of linear double stranded substrate and reacted for 30 minutes at 37° C. As shown in FIG. 15, only a single target site in the RUNX1 gene (RUNX1 site 1) supported robust cleavage by Cas-beta2. Next, RUNX1 site 1 and WTAP site 1 substrate cleavage was evaluated using capillary electrophoresis (Stebbins, M. et al. (1997) J. Chromatogr., B, Biomed. Sci. Appl. 697:181-188 and Huai et al. (2017) Nat. Commun. 8:1375). For this, RUNX1 and WTAP sites were 5′ end labeled with FAM (non-target strand) and ROX (target strand) using PCR with dye-labeled primers and oligo templates synthesized at IDT. 20 nM of the resulting double stranded DNA substrates (FIG. 16)were then digested with either 300 nM of Cas-beta2 and 3 RNP at 37° C. Aliquots were taken at 0, 1, 5, 10, 15 and 30 minutes and subject to denaturing capillary electrophoresis to distinguish between non-target strand (NTS) and target strand (TS) cleavage using the respective fluorophore. Under these conditions, both Cas-beta 2 and 3 cleaved RUNX1 site 1 while WTAP site 1 failed to produce appreciable levels of cleavage (FIGS. 17A and B). The effect of temperature on cleavage of both targets was next evaluated. When incubated at 47° C., Cas-beta3 cleaved both substrates while cleavage of WTAP site 1 by Cas-beta2 remained recalcitrant (FIGS. 18A and B). Next, the impact of supercoiling on double stranded DNA target recognition and cleavage was evaluated. For this, cleavage rates at both sites were evaluated with Cas-beta2 using supercoiled plasmid DNA (generated by cloning RUNX1 site 1 and WTAP site 1 into a plasmid DNA (e.g. puc18)) and the same plasmid DNA linearized by Bsal restriction endonuclease (NEB). Reactions were performed at 37° C. with 100 nM Cas-beta2 RNP and 2.5 nM of each substrate. As shown in FIG. 19, Cas-beta2 robustly cleaved both target sites when presented in a supercoiled state and only cleaved the RUNX1 site 1 in a linear form. The ability of Cas-beta2 and 3 to broadly cleave linear substrates was next evaluated. First, ten additional targets were selected in the WTAP and RUNX1 genes (SEQID NOs: 109-118) and surrounding regions PCR amplified for use as substrate. Next, using a sgRNA with a 20 nt length spacer, digestion reactions were performed with 50 nM of RNP and 5 nM of substrate for 30 min. For Cas-beta2, these were carried-out at 37° C. while for Cas-beta3, digestions were executed at two temperatures, 37° C. and 47° C. As shown in FIG. 20A, Cas-beta2 RNP complex cleaved 5 of the 10 targets (WTAP sites 6 and 7 and RUNX1 sites 3, 4 and 7). At 37° C., Cas-beta3 only robustly cleaved WTAP site 7 while at 47° C., it cut 8 of the 10 targets (FIG. 20B). The effect of sgRNA spacer length was evaluated next. Here, a subset of 4 WTAP and RUNX1 targets were selected (WTAP targets 6 and 7 and RUNX targets 3 and 5) for closer examination. Next, sgRNAs comprising spacers lengths of 18, 20, 22 and 24 nts for each target were T7 transcribed. Digestion reactions were then performed with each of the sgRNAs and the percentage of cleavage calculated. As averaged across all target sites, Cas-beta2 preferred a sgRNA with a 20 nt length spacer when using linear double stranded DNA substrate (FIG. 21). This can be contrasted with Cas-beta3 that was more tolerant of sgRNAs with longer spacer lengths (FIG. 22).

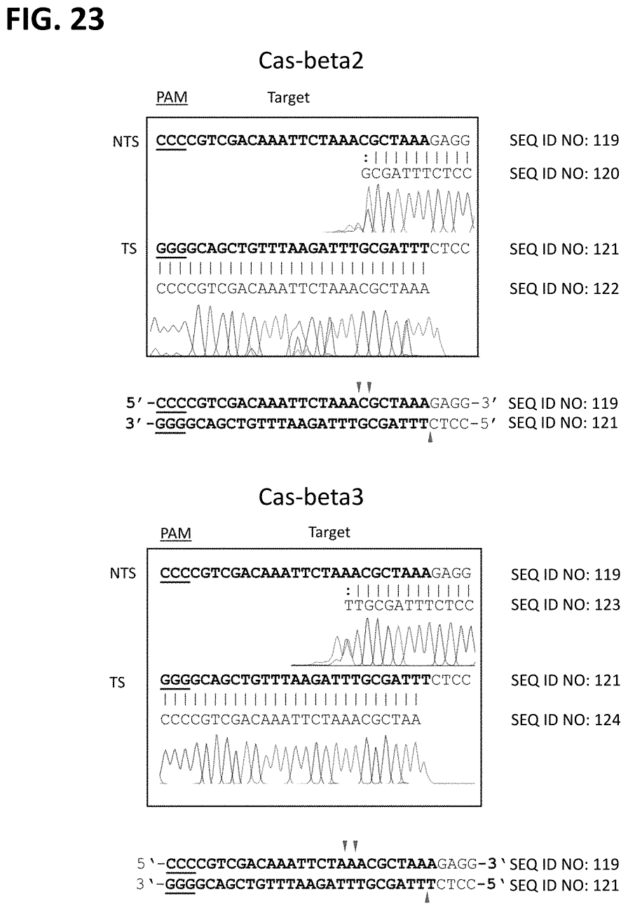

The DNA termini resulting from Cas-beta2 and 3 double stranded DNA target cleavage was examined next. For this, run-off sequencing was performed on cleaved plasmid DNA substrates as described earlier (Karvelis et al. 2020). As shown in FIG. 23, both nucleases left 5′ overhanging termini that varied in length from 6-8 nts upon target cleavage.

### Example 7

### Cas-Beta Collateral Single Stranded DNA Nuclease Activity

In this Example, the molecular features that impart Cas-beta non-specific single stranded DNA cleavage activity are identified.

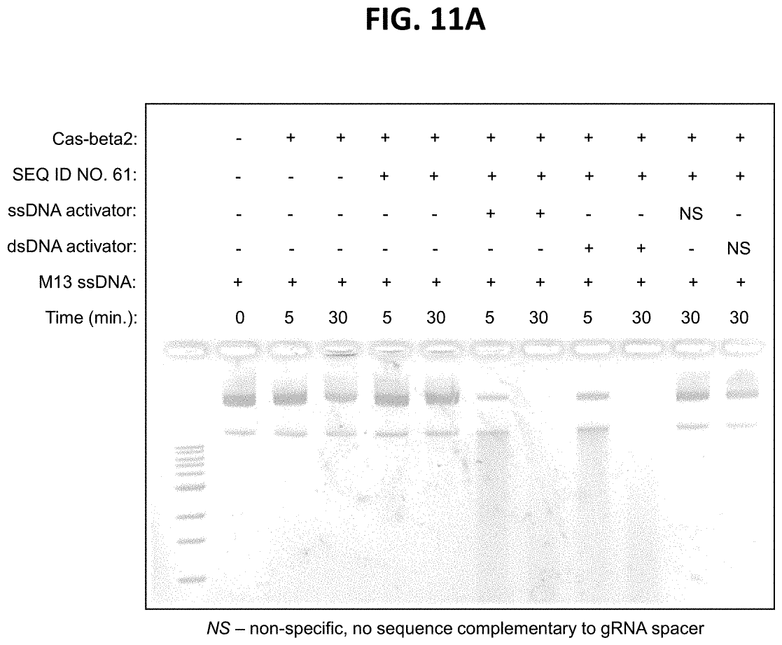

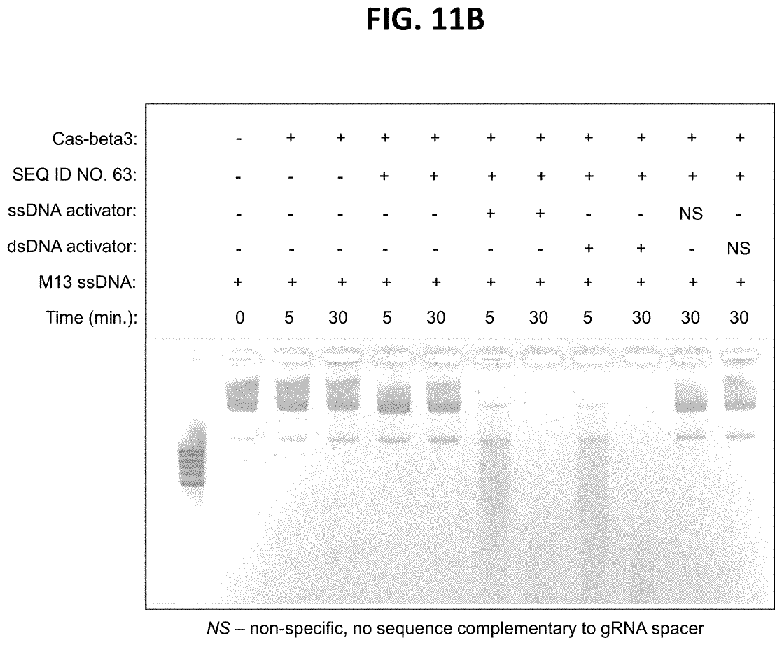

To examine the indiscriminate single stranded (ss) DNA nuclease activity of Cas-beta endonucleases, 100 nM of Cas-beta RNP was reacted with 100 nM of either single stranded (ss) DNA or double stranded (ds) DNA target in reaction buffer (1× CutSmart, NEB) in combination with 5 nM of M13 ssDNA (NEB) at 37° C. The ssDNA template was synthesized as an oligonucleotide (IDT) (SEQID NO:67) and the dsDNA substrate was generated by annealing SEQID NO:67 with SEQID NO:68 to form the dsDNA target. Both substrates contained perfect complementation to the spacer of the T2 sgRNA (24 nt spacer) (SEQID NO:61 and 63). In the case of the dsDNA activator, the non-complementary strand of the target contained a 5′-CCC-3′ PAM immediately upstream of the T2 target. To confirm that non-discriminatory ssDNA cleavage resulted from target recognition, another reaction was assembled with a non-specific (NS) ssDNA (SEQID NO:68) or dsDNA (annealed oligos SEQID NO:69 and 70) that couldn't base pair with the spacer of the sgRNA. Reactions were quenched at 5 and 30 min. and analyzed by agarose gel electrophoresis as described in Example 6. As shown in FIGS. 11A and B, Cas-beta2 and 3 RNP complexes completely degraded the M13 ssDNA after 30 min. only in the presence of a ss or ds DNA substrate that contained complementarity to the spacer of the sgRNA.

### Example 8

### DNA Expression Cassettes for Targeted Cas-Beta Genome Modification

In this Example, methods to optimize polynucleotide cassettes for the expression of Cas-beta protein(s) and guide RNA(s) (gRNA) in a cell are described. In some aspects, said cell is an eukaryotic cell. In one example of an eukaryotic cell, a Zea mays plant cell is used.

In one method, a gene encoding the Cas-beta protein was plant codon optimized and gene destabilizing features (for example but not limited to MITEs (Miniature Inverted-repeat Transposable Elements) were removed. The resulting open-reading-frame (ORF) was then optionally fused in-frame to the end of a gene also conditioned for expression that encodes a Cas protein(s) capable of RNA guided recognition of a double stranded DNA target site. In some cases, the cas gene can encode a functional, impaired, or dead nuclease. In other circumstances, additional sequences encoding an accessory domain(s) that modulate gene transcriptional states (Gilbert, L. et al. (2013) Cell. 154:442-451 and Lei, Y. et al. (2017) Nat. Commun. 8:16026), enable efficient and precise target modification (Komor, A. et al. (2016) Nature. 533:420-424, Komor, A. et al. (2017) Science Advances. 3:eaao4774, Gaudelli, N. et al. (2017) Nature. 551:464-471 and Anzalone, A. et al. (2019) Nature. 576:149-157) and manipulation of nuclear chromosomal architecture (Morgan, S. et al. (2017) Nat. Commun. 8:15993) are also optimized for expression and attached to the cas-beta gene. An intron can also be inserted into the resulting cas-beta gene to eliminate expression in E. coli or Agrobacterium prior to plant transformation. Additionally, to facilitate efficient entry into the nucleus, a sequence encoding one or more nuclear localization signal(s) (NLS) (for example but not limited to a Simian Virus 40 NLS (SEQID NO:71)) was incorporated into the Cas-beta containing ORF.

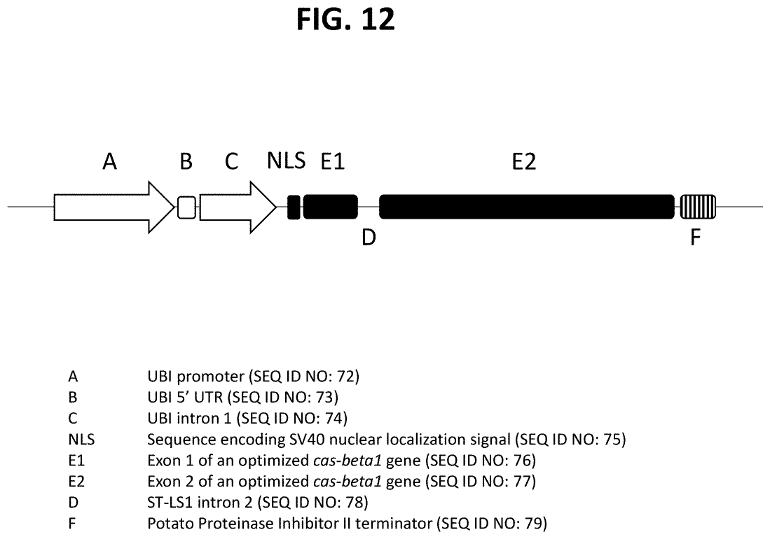

The resulting genes engineered for plant cell expression and nuclear localization were next synthesized (for example but not limited to GenScript, Inc.) and operably cloned into a plasmid DNA vector containing an upstream promoter and downstream terminator using standard techniques (for example but not limited to Golden Gate or Gibson assembly (Potapov, V. et al. (2018) ACS Synth. Biol. 7:2665-2674 and Gibson, D. et al. (2010) Science. 329:52-56). In some instances, the promoter can be a constitutive, spatially or temporally regulated, or inducible. An example of a plant optimized Cas-beta expression construct is illustrated in FIG. 12.

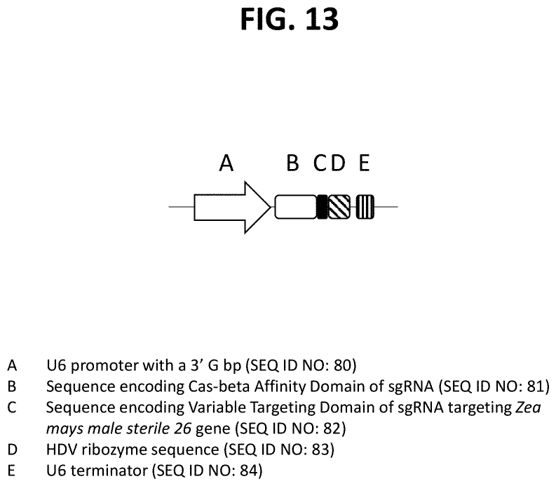

The Cas-beta protein is directed by small RNAs (referred to herein as guide RNAs) to recognize double stranded DNA target sites. These guide RNAs (gRNAs) comprise a sequence that associates with the Cas-beta protein (termed the Cas-beta affinity domain) and a region that serves to direct DNA target recognition by base pairing with one strand of the DNA target site (termed the variable targeting domain). To transcribe small RNAs necessary for directing Cas-beta to targets in plant cells, a U6 polymerase III promoter and terminator were first isolated from a plant genome and operably fused to the ends of a suitable guide RNA. To promote optimal transcription of the guide RNA from the U6 polymerase III promoter, a G nucleotide is added to the 5′ end of the sequence to be transcribed. Polymerase II promoters (for example but not limited to those listed for Cas-beta expression) in combination with a ribozyme motif, RNase P and Z cleavage sites, Csy4 (Cas6 or CasE) ribonuclease recognition site, or heterologous RNase can also be used to express the guide RNA. Moreover, the RNA processing provided by these strategies can be harnessed to express multiple guide RNAs from either a single polymerase II or III promoter. An example of a plant optimized gRNA expression construct is illustrated in FIG. 13.

### Example 9

### Delivery of Cas-Beta Components for Genome Editing

In this example, methods for introducing Cas-beta endonucleases and guide polynucleotide(s) into cells are described. In some aspects, said cell is an eukaryotic cell. In one example of a eukaryotic cell, a Zea mays plant cell is used. In other examples of a eukaryotic cell, a HEK293 human cell is used.

Zea mays Transformation

### Particle-Mediated Delivery of DNA Expression Cassettes

Particle gun transformation of 8 to 10-day-old immature maize embryos (IMEs) in the presence of baby boom (bbm) and wuschel2 (wus2) genes can be carried-out as described in Svitashev et al. (2015) Plant Physiology. 169:931-945. Briefly, DNA expression cassettes are co-precipitated onto 0.6 μM (average size) gold particles utilizing TransIT-2020. Next, the DNA coated gold particles are pelleted by centrifugation, washed with absolute ethanol and re-dispersed by sonication. Following sonication, 10 μl of the DNA coated gold particles are loaded onto a macrocarrier and air dried. Next, biolistic transformation is performed using a PDS-1000/He Gun (Bio-Rad) with a 425 pound per square inch rupture disc. Since particle gun transformation can be highly variable, a visual marker DNA expression cassette encoding a yellow fluorescent protein (YFP) is also co-delivered to aid in the selection of evenly transformed IMEs. To determine the plant transformation culture conditions optimal for Cas-beta and associated guide polynucleotide activity, transformed IMEs are incubated at 28° C. for 48 hours, or at a range of temperatures lower or higher than 28° C. to establish the temperature optimum for target DNA cleavage and repair. Post-bombardment culture, selection, and plant regeneration were performed using methods described previously (Gordon-Kamm, W. et al. (2002) Proc Natl Acad Sci USA. 99:11975-11980) except bbm and wus2 genes were expressed with non-constitutive promoters, maize phospholipid transferase protein (Zm-PLTP) and maize auxin-inducible (Zm-Axigl) promoters, respectively (Lowe, K. et al. (2018) In Vitro Cellular Dev-Pl. 54:240-252).

### Particle-Mediated Ribonucleoprotein Delivery

Cas-beta endonuclease and associated guide polynucleotide(s) ribonucleoprotein (RNP) complex(es) can be delivered by particle gun transformation as described in Svitashev, S. et al. (2016) Nat. Commun. 7:13274. Briefly, RNPs (and optionally DNA expression cassettes) are precipitated onto 0.6 mm (average diameter) gold particles (Bio-Rad) using a water soluble cationic lipid TranslT-2020 (Minis) as follows: 50 ml of gold particles (water suspension of 10 mg/ml) and 2ml of TranslT-2020 water solution are added to the premixed RNPs (and optionally DNA expression vectors), mixed gently, and incubated on ice for 10 min. RNP/DNA-coated gold particles are then pelleted in a microfuge at 8,000g for 30 s and supernatant is removed. The pellet is then resuspended in 50 ml of sterile water by brief sonication. Immediately after sonication, coated gold particles are loaded onto a microcarrier (10 ml each) and allowed to air dry. Immature maize embryos, 8-10 days after pollination, are then bombarded using a PDS-1000/He Gun (Bio-Rad) with a rupture pressure of 425 pounds per inch square. Post-bombardment culture, selection, and plant regeneration are performed using methods described previously (Gordon-Kamm, W. et al. 2002) except bbm and wus2 genes were expressed with non-constitutive promoters, Zm-PLTP and Zm-Axigl promoters, respectively (Lowe, K. et al. 2018).

### Agrobacterium-Mediated Transformation

Agrobacterium-mediated transformation is performed essentially as described in Djukanovic et al. (2006) Plant Biotech J. 4:345-57 except bbm and wus2 genes were expressed with non-constitutive promoters, Zm-PLTP and Zm-Axigl promoters, respectively (Lowe, K. et al. 2018). Briefly, 10-12 day old immature embryos (average 2 mm in length) are dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HC1, 1.5 mg/L 2, 4-D, 0.690 g/L L-proline, 68.5 g/L sucrose, 36.0 g/L glucose, pH 5.2). After embryo collection, the medium is replaced with 1 ml Agrobacterium at a concentration of 0.35-0.45 OD550. Maize embryos are incubated with Agrobacterium for 5 min. at room temperature, then the mixture is poured onto a media plate containing 4.0 g/L N6 Basal Salts (Sigma C-1416), 1.0 ml/L Eriksson's Vitamin Mix (Sigma E-1511), 1.0 mg/L thiamine HC1, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos are incubated axis down, in the dark for 3 days at 21° C., then incubated 4 days in the dark at 28° C. at which time they may be harvested for DNA extraction.

In another variation for stable transformation, the embryos are then transferred onto new media plates containing 4.0 g/L N6 Basal Salts (Sigma C-1416), 1.0 ml/L Eriksson's Vitamin Mix (Sigma E-1511), 1.0 mg/L thiamine HC1, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos are subcultured every three weeks until transgenic events are identified. Somatic embryogenesis is induced by transferring a small amount of tissue onto regeneration medium (4.3 g/L MS salts (Gibco 11117), 5.0 ml/L MS Vitamins Stock Solution, 100 mg/L myo-inositol, 0.1 uM ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots are transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 28° C. One week later, plantlets are moved into glass tubes containing the same medium and grown until they were sampled and/or transplanted into soil.

In another variation, leaf tissue can be directly transformed with Cas-beta and guide polynucleotide expression cassettes. In this situation, enhancers and promoters that enable constitutive expression (for example but not limited to Ubiquitin, Figwort Mosaic Virus (FMV) enhancer, Peanut Chlorotic Streak Caulimovirus (PCSV) enhancer and Mirabilis Mosaic Virus (MMV) enhancer) are used to drive expression of bbm and wus genes.

### HEK293 Transformation

### Cell Culture Lipofection

HEK293 (ATCC) cells are cultured in DMEM (Gibco) with 10% FBS (Gibco) and penicillin/streptomycin (Gibco) at 37° C. in 5% CO2. A day prior to transfection cells are seeded in 96-well plates at 3.6×10⁴ density. NLS-tagged Cas-beta RNP complex is assembled by mixing 20 pmol of purified protein with 40 pmol of sgRNA in 25 μl Opti-MEM (Gibco) and incubated at room temperature for 30 min. After complex assembly 25 μl of Opti-MEM, containing 1.2 μl Lipofectamine 3000 (Thermo Fisher Scientific) is added and the mixture is incubated for an additional 15 min. at room temperature before transfection of the cells. Genomic DNA is extracted 72 h after transfection using QuickExtract DNA Extraction Solution (Lucigen) and regions surrounding target sites evaluated for the presence of mutations indicative of DNA double strand break and repair.

### Cell Culture Electroporation

NLS-tagged Cas-beta RNPs are electroporated into HEK293 (ATCC Cat# CRL-1573) cells using the Lonza 4D-Nucleofector System and the SF Cell Line 4D-Nucleofector® X Kit (Lonza). For each electroporation, RNPs are formed by incubating 100 pmoles of sgRNA with 50 pmoles of Cas-beta protein in nucleofector solution in a volume of 17 μL at room temperature for 20 minutes. HEK293 cells are released from culture vessels using TrypLE™ Express Enzyme 1X (ThermoFisher) washed with 1X PBS without Ca++ or Mg++ (ThermoFisher) and counted using a LUNA™ Automated Cell Counter (Logos Biosystems). For each electroporation, 1×10\hat{ }5 live cells are resuspended in 9 μL electroporation solution. Cells and RNP are mixed and transferred to one well of a 16-well strip and electroporated using the CM-130 program. 75 μL of pre-warmed culture is added to each well and 10 μL of the resultant resuspended cells are dispensed into a well of a 96-well culture vessel containing 125 μL of pre-warmed culture medium. Electroporated cells are incubated at 37° C., 5% CO2 in a humidified incubator for 48-96 hours before analysis of genome editing.

### Example 10

### Detection of Double Strand DNA Target Cleavage in Eukaryotic Cells

In this example, a method for detecting the functional formation of a novel Class 2 endonuclease (Cas-beta) and associated guided RNA(s) (polynucleotide(s)) complex in cells are described. In some aspects, said cell is an eukaryotic cell. In one example of an eukaryotic cell, a Zea mays plant cell is used. In another example of a eukaryotic cell, a HEK293 human cell is used.

The functional formation of a Cas-beta nuclease and guide RNA(s) complex in eukaryotic cells is monitored by examining one or more different chromosomal DNA target sequences for the presence of insertion and deletion (indel) mutations indicative of DNA target site double stranded cleavage and cellular repair. This is carried-out by targeted deep sequencing as described in described in Karvelis, T. et al. (2015) Genome Biology. 16:253 (Methods Section: in planta mutation detection) or other equivalent methods devised to detect alterations in DNA (for example but not limited to Guschin, D.Y. et al. (2010) In: Mackay J., Segal D. (eds) Engineered Zinc Finger Proteins. Methods in Molecular Biology (Methods and Protocols), vol 649. Humana Press, Totowa, N.J.). Briefly, for Zea mays, the 20-30 most evenly transformed immature embryos (IEs), based on their fluorescence, are harvested two days after transformation for each experiment. Next, total genomic DNA is extracted and the region surrounding the intended target site is PCR amplified with Phusion® HighFidelity PCR Master Mix (New England Biolabs, M0531L) adding on the sequences necessary for amplicon-specific barcodes and Illumina sequencing using “tailed” primers through two rounds of PCR and deep sequenced. The resulting reads are then examined for the presence of mutations at the expected site of cleavage. To validate the targeted changes detected in our experimentation as true chromosomal mutations, an informatic approach to account for background alterations resulting from technical approaches (e.g. PCR and NGS) can also be applied. For this, the frequency of each mutation detected in test experiments is compared with those found in the negative control (experiments setup without the Cas-beta nuclease or guide RNA). Then, if the difference between test and control experiments are significant (30 or more times greater in the test experiment), they are considered as true mutations. Additionally, to accurately report our findings and place them in the context of background technical variation, bona fide mutations from test experiments that are also found in the negative control (albeit at a much lower read count) are also reported.

For Zea mays plants regenerated from transformation experiments, a similar method is followed except total genomic DNA is extracted from leaf punches.

For HEK293, a similar process is performed except the cell culture was harvested 48 to 120 hours after transformation and cell lysate was used as template in PCR.

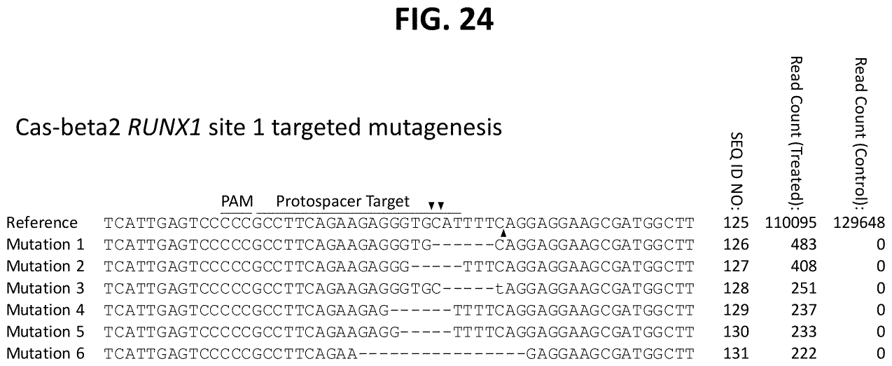

When nucleofected as NLS-tagged RNP into HEK293 cells, Cas-beta2 produced targeted chromosomal DNA sequence alterations when paired with a sgRNA targeting RUNX1 site 1 (SEQID NO:143) (FIG. 24). These comprised deletion mutations that coincided with the expected cut-sites of Cas-beta2 and disrupted protospacer target recognition (FIG. 24). The absence of similar mutations in control experiments confirmed that the changes were due to the introduction of Cas-beta2 RNP, altogether, providing the first evidence for double stranded DNA target binding and cleavage activity in a eukaryotic cell (FIG. 24).

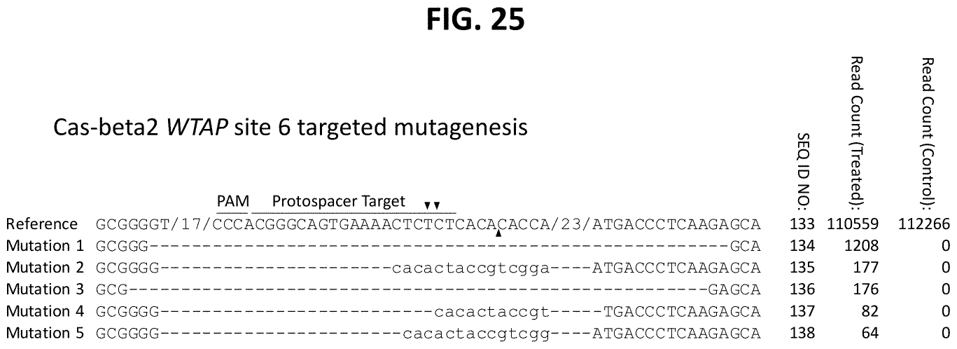

Two additional HEK293 chromosomal targets, WTAP sites 6 and 7 (SEQID NOs: 112 and 113), that showed robust cutting in vitro (FIG. 21) were also targeted for cleavage with Cas-beta2. Here, Cas-beta2 sgRNAs (SEQID NOs: 144 and 145) capable of base pairing with the complementary strand of WTAP sites 6 and 7 were generated by T7 in vitro transcription, complexed with Cas-beta2 NLS-tagged protein, and delivered into HEK293 cells using electroporation. Then after 48 hrs, the WTAP sites were examined for evidence of double strand break repair. As shown FIG. 25, deletion mutations spanning the expected Cas-beta2 cut-sites were recovered. Taken together, this corroborates the finding that Cas-beta2 can bind to, cleave and introduce targeted genomic modifications in eukaryotic cells.

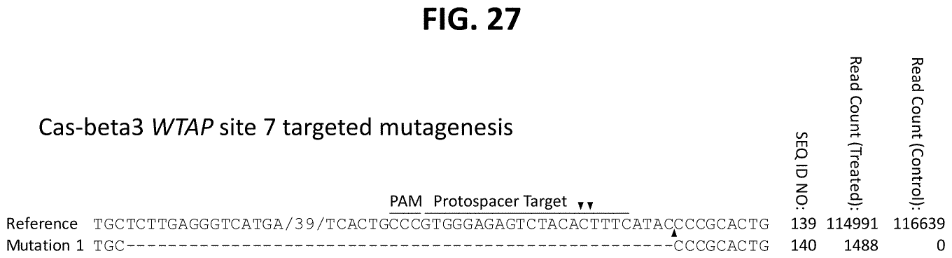

Next, Cas-beta3 was tested for its ability to introduce targeted chromosomal changes at WTAP site 7 in HEK293 cells. When Cas-beta3 and sgRNA (SEQID NO: 146) were delivered into HEK293 cells (as RNP), deletion mutations spanning the WTAP 7 target site were recovered (FIG. 27). In all, this shows that orthologous Cas-beta nucleases can also be used to recognize, cleave, and modify eukaryotic chromosomal DNA targets.

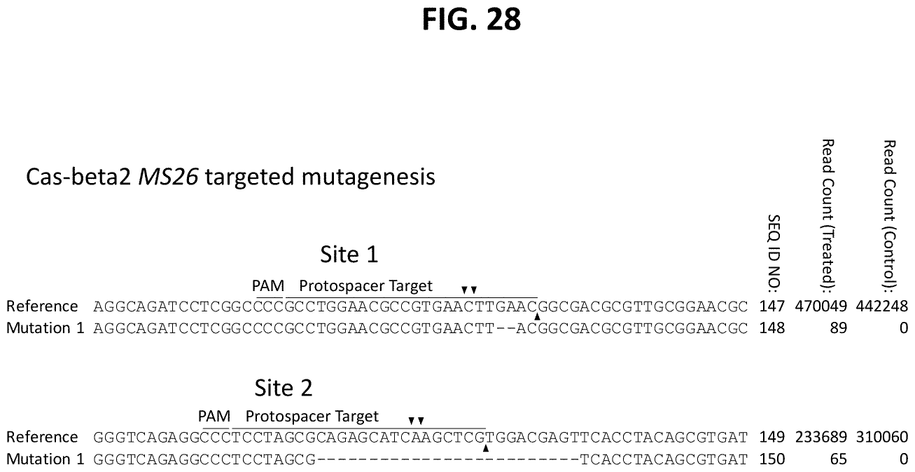

To establish that Cas-beta can function in other eukaryotic cells, its ability to recognize and cleave Zea mays chromosomal DNA target sites was assessed. When expression cassettes encoding Cas-beta2 and sgRNA were delivered into IEs using biolistics, targeted mutations were recovered at both male sterile 26 (MS26) gene targets two days after transformation (FIG. 28), altogether, showing that Cas-beta nucleases exhibit broad functionality in different eukaryotic cell types.

### Example 11

### Detection of Double Strand DNA Target Cleavage in Prokaryotic Cells

In this example, a method for examining the functional formation of a Cas-beta nuclease and associated guided RNA(s) (polynucleotide(s)) complex in prokaryotic cells are described. In some aspects, said cell is a heterologous prokaryotic cell. In one example of a prokaryotic cell, a E. coli cell is used.

One method to assess Cas-beta double stranded DNA target cleavage is to examine its ability to interfere with plasmid DNA transformation in E. coli cells (Burstein, D. et al. (2017) Nature. 542:237-241 and Karvelis, T. et al. 2020). Here, a double stranded plasmid DNA comprising a selectable marker (for example but not limited to streptomycin) and a Cas-beta target site(s) (a region capable of base pairing with the CRISPR RNA that is in the vicinity of a protospacer adjacent motif (PAM)) is transformed into E. coli (ArcticExpress DE3 or equivalent) that contains a Cas-beta endonuclease and guide RNA expression cassette, by methods known in the art (for example but not limited to electroporation). In one example guide RNAs are expressed from the “complete locus” or “minimal locus” containing a CRISPR array (see FIG. 5). In another example, a separate T7 promoter is used to drive sgRNA expression. In the absence of double stranded DNA target cleavage, many cells containing the plasmid and antibiotic resistance marker are recovered by growth on selective media. In contrast, double stranded DNA target cleavage of the in-coming plasmid DNA results in a reduction or interference in the recovery of resistant cells. Experiments setup with plasmids that don't contain a Cas-beta target site, “no target”, provide a baseline for transformation efficiency. Also, interference experiments can be performed with and without induction of Cas-beta or guide RNA expression (for example IPTG (0.1 mM)) to examine target cleavage under different Cas-beta endonuclease and guide RNA expression conditions. Inductor can also be used in multiple steps before and after transformation to promote Cas-beta endonuclease and guide RNA expression. This includes when cells reach an OD600 of —0.5, in recovery media following transformation and in solid agar plates containing appropriate antibiotics. To assess plasmid DNA interference, transformations can also be diluted in 10-fold increments, spotted on selective media, grown overnight at 37° C., and inspected for bacterial colony growth.

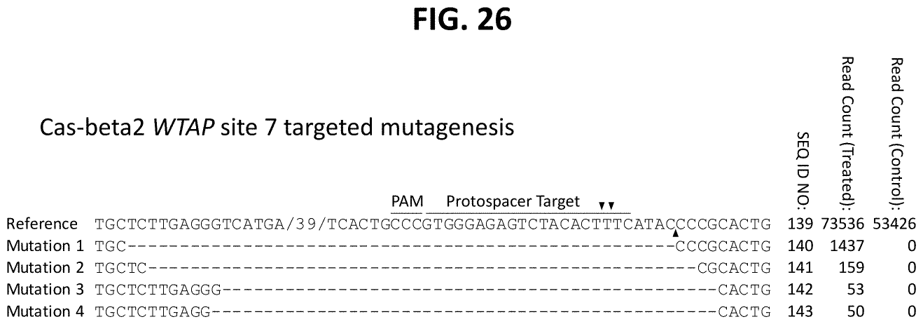

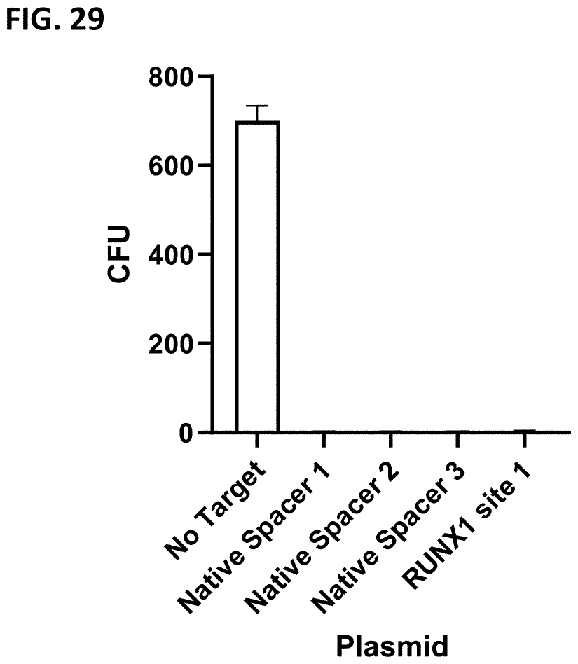

When the recipient strain, E. coli Arctic Express (DE3), carrying Cas-beta2 nuclease and guide RNA expression cassettes was transformed with the target plasmid, no colonies were obtained (FIG. 29). This can be contrasted with the control plasmid, that lacked a protospacer target, which yielded—700 colonies (FIG. 29). To ensure reproducibility, experiments were performed minimally three times and with different targets (native spacer 1 (SEQID NO:151), native spacer 2 (SEQID NO:152), native spacer 3 (SEQID NO:153) and RUNXJ site 1 (SEQID NO:95)) (FIG. 26). Taken together, this shows that Cas-beta2 and associated guide RNA function to bind to and cleave double stranded DNA targets in heterologous prokaryotic cells.

### Example 12

### Sequence Specific Detection of Nucleic Acids

In this example, a method for detecting a nucleic acid polynucleotide is described. In some aspects, said detection is for a single or double stranded DNA polynucleotide.

In one method, the collateral non-specific degradation of single stranded DNA triggered by Cas-beta DNA target site recognition (see Example 7) is used to produce a signal indicating the presence of said target site. Here, single stranded DNA molecules that do not contain a Cas-beta target sequence are first engineered to release a 5′ fluorophore (for example 6-carboxyfluorescein (FAM) or hexachloro-fluorescein (HEX)) from a 3′ quenching moiety (for example tetramethylrhodamine (TAMRA) or Black Hole Quencher 1 (BHQ-1)) (Li, SY. et al. (2018) Cell Discov. 4:20). The quenching moiety can also be replaced with a molecule (for example biotin) that impedes migration through a lateral flow strip designed to spatially separate intact and degraded single stranded DNA (Broughton, J. et al. (2020) Nat Biotech. 38:870-874). Collateral single stranded DNA substrates may also be modified to contain a thiol group and optionally a redox moiety (for example ferrocene) and affixed to gold electrodes so that non-discriminatory cleavage following Cas-beta target detection can be identified using electrochemistry similar that described for DNase I (Sato, S. et al. (2008) Anal. Biochem. 381:233). Next, the single stranded DNA substrates engineered to elicit a signal upon degradation are combined with a test DNA mixture and allowed to react with Cas-beta and sgRNA ribonucleoprotein (RNP) complexes using biochemical parameters conducive for Cas-beta nucleolytic activity (see Examples 6 and 7). Reactions are then assayed for evidence of single strand DNA degradation triggered by Cas-beta DNA target site recognition.

In another method, the binding of a Cas-beta sgRNA RNP complex to a target DNA sequence is used to elicit a signal in a graphene biosensor (Hajian, R. et al. (2019) Nat. Biomed. Eng. 3:427-437). For this, a cleavage inactivated or dead (d) Cas-beta RNP was first generated by substituting alanine residues into either position 528 (SEQID NO: 154) or 634 (SEQID NO: 155) of the Cas-beta2 nuclease. Then, by affixing dCas-beta2 RNP to graphene sensors, the selective binding of a DNA target site is detected in a test mixture by measuring the current potential between gating electrodes.

1. A synthetic composition comprising:
(a) a Cas endonuclease or a functional fragment thereof, comprising an amino acid sequence that is at least 90% identical to a sequence selected from the group consisting of SEQID NOs: 13, 14, 15, and 57,
(b) a target double-stranded DNA polynucleotide, wherein the target double-stranded DNA polynucleotide is heterologous to the source of the Cas endonuclease, and
(c) a guide polynucleotide comprising a variable targeting domain that comprises a region of complementarity to the target double-stranded DNA polynucleotide;
wherein the Cas endonuclease or the functional fragment thereof recognizes a PAM sequence on the target double-stranded DNA polynucleotide, wherein the guide polynucleotide and the Cas endonuclease or the functional fragment thereof, form a complex that binds to the target double-stranded DNA polynucleotide, wherein the Cas endonuclease or the functional fragment thereof comprises DNA binding activity.

2. The synthetic composition of claim 1, wherein the Cas endonuclease is provided as a polynucleotide encoding the Cas endonuclease.

3. The synthetic composition of claim 1, wherein the Cas endonuclease cleaves the double-stranded DNA polynucleotide.

4. The synthetic composition of claim 1, further comprising a heterologous polynucleotide, wherein the heterologous polynucleotide is an expression element, a transgene, a donor DNA molecule, or a polynucleotide modification template.

5.-8. (canceled)

9. A synthetic composition comprising:
(a) a Cas protein or functional fragment thereof, comprising an amino acid sequence that is at least 90% identical to a sequence selected from the group consisting of SEQID NOs: 13, 14, 15, and 57, (b) a target double-stranded DNA polynucleotide, wherein the target double-stranded DNA polynucleotide is heterologous to the source of the Cas protein, and
(c) a guide polynucleotide comprising a variable targeting domain that comprises a region of complementarity to the target double-stranded DNA polynucleotide;
wherein the Cas protein recognizes a PAM sequence on the target double-stranded DNA polynucleotide, wherein the guide polynucleotide and the Cas protein form a complex that binds to the target double-stranded DNA polynucleotide;
wherein the Cas protein comprises DNA binding activity and lacks double-strand DNA cleavage activity.

10. The synthetic composition of claim 1, further comprising a deaminase.

11. The synthetic composition of claim 1, wherein the Cas endonuclease is part of a fusion protein.

12. The synthetic composition of claim 11, wherein the fusion protein further comprises a heterologous nuclease domain.

13. The synthetic composition of claim 1, further comprising a eukaryotic cell, wherein the eukaryotic cell is a plant cell, an animal cell, or a fungal cell.

14. (canceled)

15. The synthetic composition of claim 13, wherein the plant cell is a monocot cell or a dicot cell.

16. The synthetic composition of claim 13, wherein the plant cell is from an organism selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, Arabidopsis, safflower, and tomato.

17. A construct comprising a polynucleotide encoding a Cas protein operably linked to a heterologous expression element, wherein the Cas protein shares at least 90% sequence identity to a sequence selected from the group consisting of SEQID NOs: 13, 14, 15, and 57, and wherein the Cas protein comprises DNA binding activity.

18. The synthetic composition of claim 1, wherein at least one component is attached to a solid matrix.

19. A method of introducing a targeted edit in a target polynucleotide of a cell, the method comprising providing a heterologous composition to the cell, wherein the heterologous composition comprises:
(a) a Cas endonuclease or a functional fragment thereof, comprising a polypeptide at 90% identical to a sequence selected from the group consisting of SEQID NOs: 13, 14, 15, and 57,
wherein the Cas endonuclease recognizes a PAM sequence on the target polynucleotide, and wherein the Cas endonuclease comprises DNA binding activity; and
(b) a guide polynucleotide comprising a variable targeting domain that is substantially complementary to a portion of the target polynucleotide, wherein the guide polynucleotide and Cas endonuclease form a complex that can recognize and bind to the target polynucleotide.

20. The method of claim 19, further comprising introducing at least one nucleotide modification in the genome of at least one cell of the organism as compared to the target sequence of the genome of the cell prior to the introduction of the heterologous composition, further comprising incubating the cell and generating a whole organism from the cell, and ascertaining the presence of the at least one nucleotide modification in the genome of at least one cell of the organism as compared to the target sequence of the genome of the cell prior to the introduction of the heterologous composition.

21. A progeny of the organism obtained by the method of claim 20, wherein the progeny comprises the Cas endonuclease or functional fragment thereof.

22. The method of claim 19, wherein the cell is a eukaryotic cell.

23. (canceled)

24. The method of claim 22, wherein the plant is a monocot or a dicot.

25. The method of claim 22, wherein the plant is selected from the group consisting of: maize, soybean, cotton, wheat, canola, oilseed rape, sorghum, rice, rye, barley, millet, oats, sugarcane, turfgrass, switchgrass, alfalfa, sunflower, tobacco, peanut, potato, Arabidopsis, safflower, and tomato.

26. The method of 19, further comprising introducing a heterologous polynucleotide, wherein the heterologous polynucleotide is a donor DNA molecule or a polynucleotide modification template that comprises a sequence at least 50% identical to a sequence in the cell.

27. (canceled)


SEQUENCE LISTING (155 sequences)

SEQ ID NO: 1 - protein, 337 aa, Armatimonadetes bacterium
MVDGYGSQVLKHSERLVLRIPSALDKNIKREVPVLHLDHLLVGTKGVLVSSDALALCCERGIPVTVVDWRGRPVGRFGSPALHGSAHIRRAQLEAFDANLGAEFAREVVCGKLLNQADNLRYFGKNRKTRDPAQHELLETSADDINEISKRASCISEKCANTARLPLMTLEAEGARIYWSALSRLYGTRSGFARREQRGTRDPVNAALNYAYGVLNGEVWNAVVLAGLEPYAGLLHVDRPGRLSFVLDLMEEFRPIIADRLVFGLAAKGWKIGQEENGWLDFATKQRLLKAISERWDARVNYQGRKIRLRSVLQLQARDAARHFLGRAQYRAFRQRW

SEQ ID NO: 2 - protein, 350 aa, Armatimonadetes bacterium
MRYEIVDGYGCQVLKHSERLVLRYPLAPPGERGPGGEGKQPKREVPVLHLDHLLIGTKGVTVSTDALALCCERGIPVTVVDWRGRPVGRFGSPALHGTAQVRRAQIAAFATEIGATFAREVVSGKLLNQADNLRYIGKNRKTRAPNEHEALTRTADTLQRLAKKAATVKGKNADDVRLPLMTVEAEGARAYWSVLSEVYGERSGFAKREQRGTRDPVNAALNYAYGVLNGEVWNATILAGLEPYAGFLHVDRPGRLSFVLDLMEEFRPVVADRVVFGLVAKGWKIGQEENGWLDMPTKRRLIQAIGERWGARVLHQARKLQLRSVLQLQARDAARHFQGKAEYIAFRLRW

SEQ ID NO: 3 - protein, 343 aa, Armatimonadetes bacterium
MVKYEYVETFGSAVQKHSERLVVSEPSGEGGQRTKRQVPALHLDHLLIGSRGVSISSDALELCCERGIPVTIVDRRGKPVGKFTAPAIHGTSRTRRAQIRAYENGLGVTFARSVVIGKAANQAINLKYFAKNRRERSPDQYETLRKSAEAIDRVARRAKKISANCIDEVRQPLMVLEAEASRIYWSSLSALYGSSSGFVHREQRGTKNPVNAALNYAYGVLTGEVWTACLLAGLEPYAGFLHADRPGRLSFVLDLIEEFRPVVADRVVFALAAKGWRIEQEENGWLSLASKNKLLASLAERLDSPEPDRGRRRKLRNVIQRQAYAAAQHFLGNETYVPYKQRW

SEQ ID NO: 4 - protein, 92 aa, Armatimonadetes bacterium
MKWLVCYDIEKDSVRQKIADFCLDKGLERVQYSVFLGDMNQTLAFDLAAQIRRRMGDHPGQVRFIPICDRDWKKTFRIQIGNYMGVKPSNGK

SEQ ID NO: 5 - protein, 92 aa, Armatimonadetes bacterium
MKWLVCYDIEKDSVRNKVADFCLDKGLERVQYSVFLGSMTRTLAKELGAQIRRKMGKNPGQVRFVPICDKDWKTSFRVQVGDHMGEKASDGK

SEQ ID NO: 6 - protein, 82 aa, Armatimonadetes bacterium
MTWLVVYDIEDDRVRTKVADYCLDKGLERIQYSCFLGEMSRTLARELASKCKRKLGDKPGKIRLVPVCEKDLASQVRIENVP

SEQ ID NO: 7 - protein, 177 aa, Armatimonadetes bacterium
MKAGIDAEAERQRLETRRGFSQYGITAVDKRFQFSVRSDSLGLAGRIDCLIETTDVTFEEAQAGLRPNEWNWDDPLFVPVEYKTTFRVQQKHNVMQLAAYARMLESLTGTAVPFGFIVMLPEEEVLKIEISSEIKRSLDFLIEEVQRGLVSDELPRPTPHSGKCQNCEFRRFCNDVW

SEQ ID NO: 8 - protein, 221 aa, Armatimonadetes bacterium
MASSAHAYQPRDTVSVSELRQWMYCPRVVWYGRSMGDYRPTTGAMKVGIEAEAERQRLEERRSFAQYGLEACNKRFQVPVASEALGLSGRIDCLIELTPVSLEDAQVGVRPLNWKVGDPMFVPVEYKWTSRADQRQNTIQLAAYGMILESLTGTPVPLGFIALLPEEEVVRVELVPRVRRAVKCTLDEAREGLSAPELPWPTLHRGKCQDCEFRRFCNDVW

SEQ ID NO: 9 - protein, 213 aa, Armatimonadetes bacterium
MEVSPSDGFVSVSEVRQWSYCPRVVWHNRWLGERRPQTSRMEEGRADQAERERKEKRRTFAEYRLPAQSRRFNVYLRSERLGVSGVVDAVLELTNRSIDEVDSQGLDPERPYFAPVEYKSTQERVGRHHLLQLAGYAALLSDITGTSVPFGYFVSLPNGRASRVELSEKAREEFLSCVQGIRNMVVECRMPEPTPSRAKCRDCEFRRFCNDVW

SEQ ID NO: 10 - DNA, 2565 bp, Armatimonadetes bacterium
atgcccaaaaagacatcgacggtcgctctgtcacccagagatattcgcttgcgcgaacttggagagaagcgacttcaaaggttgcgacagcgcgaagagaagattcgtcgtcacctggagtcggagcgcgggcggcgtgactttcaatcgctgcactttcttcttcataaaattgaagttgaacgaaacgatctgtaccgaaatctttaccagaacgaagggcacgagtcgtatgtgccaaagccaggtaagacgaaacataggaaagaactttctttgccatccacagagttaccgtctccacctgatgagaagaaagggccccggcccaaaaagagtcgctatgtgattccccagcccgtacctggaatcaatctcccacgattgatcaatagattcggtaaatcggatcaaaagtccgaatcggatcaagaaggcagattttggacttcagcgcctttcatcgaagttgagctgcctatgcttaatgcccatcgagtcataaaggcgttgatgcgattcgttcagaaagacgagcgttcggttgtccgaacatgggctgtgaccaagtttggcagcattgaggccgccagagaagtcctgctagcaggagctttgctgcaaagagagccggaaatcatgagaggcttcctccagaatattgacccctgggggagtttgagcgatgaggaactcattcgcgatgaaaaagcgtggcggacggtgaagctcctagcccaaaagaattgggtggatcaaatcgcgaagtcgatcaaggactcggcgcctaagggcgtagataaagacactttggatcgtcgcctgcggagtggcttaaaagcattccattctgcggcaaattcaggaaaacacacgaatccccagtttccctatttgacatcggagaaaccgtccgcgaactttgaatcagttgtcgactctgtgcttgagttcctcgatctggaggacaaggatcgatacacgattgcgaaggttgacgacaagaaacgccaccgagtgacggctctgcaaaaggagctaggccaagccaaaccacgtgtaaggttggagcaggaacgtagcaggtgggctggccactcgtatctccaagggaccattaccaggaaaaggcaggcttccctcgtttgggatggtcaccgaacggagaacggtttggctctcgccatcccattagatggcatgccgaaaattgacgtgcagcgatatatgtatcaagatggcacctcccttctctcggatcggcaaattacttccaagaccaagtccgagggtaaggactgtgccttgatgcctctacgatttaagcatgcctttcttcgatggtatacgaaacacgtcgaaaatcacgtggccgaggcccctttggaacggcgatgcattcataacacaacgcagtttgtcatcgtcgacccagaaggaaagcatcctcggctgttcatccgacctgtcttcaaattctatgactctaataagacaatacagaacagtaacgccccctggtgcaaaccgcagtgtcgataccttatcggcattgatcggggcatcaactacgtgctacgagcggttgttgtagatactgaagagaaagccgtaatcgacgacatccctctaccgggtcgaaagcgggagtggcgagccattcggcaagagatcgcgtactttcagcgcatgcgggacctctccaagagtgcccaagaaaggaaccgctatgtggttgcgcttgccaaagctcgtaggaaagatcgcagcttgggcaagacagagacggttgaggctgttgcaaaacttgttcagaattgcagcgagagatttggggaaggaaactactgcttcgtgttggagaaccttgagttaggtgctcttaacttaaaaagaaataaccgagttaaacatctcgcttccatggaagaggcattgatctatcagatgcggaaaagaggctacttctacaactcccgatcgaaccgggtggatggtgttcgttgggaagccgcacgctatacaagtcaagtttctccgtttggctggtgggccaagcgcgatgaagtggagaaggccaagaaacaagataaaagcatggcgattggccgcaagattggcgagggatatgaaggtccgcaggacgatgaaatagaaagtcattcgactatctatcggcagggcagatggatgaaactcagaaatgaagaaggaaaggcctacggaagaagtcggtttgtggttcagccggaagacttggaccctgcacaacccagaaggttcagttggggaagtgaacttttctgggatccctatcaaaaggaatttaaaggaaagtccttctctcaaggcgttgtgttggatgctgattttgtgggagccctaaacattgcccttcggccactagtcaacgacggcaaaggcaaaggtttcaccaccgcgatgatggcggaagcacatgtcaagttgaacccgacctttgagatccgttgcaagatcccggtttacgaatttatcgctgagaacgacaattctcgtgccgcgctgagaaggattgtgatatag

SEQ ID NO: 11 - DNA, 2604 bp, Armatimonadetes bacterium
atgggtaagaatcgatcctcgtcctcggatttgagcccgctcgaacggtccttgcggaaggtcggtgagaatcgccttgagcggctgcgggtgcgagaggagaagattaggaagcacatagaacagcacccccgcggtaagaacgatcatcaggctctccacttcttattgcaccaaatcgaggtcgagcgtaacgacctgtaccgaaacctcaaagaccccgagtacgtgcccaaaccagcgaaacagcggcgcgaaagacggcagatcaacgtcgccaaacccccgactcgaccaaagaaggaaaaggggcctcaaccagagtcgacgaagtacgtgatccgtccaccagtccctgggaaaaaccttcctgcctttgctagcaagtacgaggcgcgagacacgcgggacgattcctaccaggacggtcgctcatggacctccgcaccatatgttgaagtcgaacttcccatccttggtgcagacaaagtcatccagaaactgatgaagttcgtgcagaaggacgagcggtcgatcgtgcgcgactgggcgacaaagacgtatagctcgatcgaagccgcaagagaagcactccttgtcggggcacaagtctcggaagacgtttcggtctggcgcggactcctcgcagaaacgaagaacgcacagaacttcgccgccctctccgacgatcagatcgaagcagcgatgtcgaaggaggcgaagggcgcggacttgcgtccgaggcgcgccgcactgctggtcgcacagcgccactgggtggatcagaccgtcaaagcaatcaaggagtccgcaccgtccggcgtcgacaaggacactctcgatcgccgtctgcgcgcaggtctgagggggtttcatactgcggccaactcaggcaagcacacgaacccgcagttcccatacctcaccgcagagaagccggtagtcccgatggagtctgttgttcagagcgtattggcctttctcgacgatccagacgatcaaaggtacacgaaggacaaagaagacgacaagaagcgccaccgcgtcactgtcttgcagaaggagctcggaaaggcgaggccacgaaaacggttagaactccaaacgccgaaatgggccggcaggcccacggtaaaaggaaccatcagcaaacggcgcgacgcagcgctcgtctgggacacaagcaaagaagcgaacgggctttgtctcgcgctcccaatcgggggcatgccgaagatagacgtcgagcagttcatctaccaggatgggacgtcgctcctgtccgattgccagatcgcatcgaaaacgaccaagaagggcgcggcttgcgcagtcttgccgctcaagcccaagcatgacttcctgcgctggttcaccaagcacgtcgagaaccacaatcccgacgctccactggaacgcaggtgcctccacaacacgacccagttcgtcatagtcgacccagaagggccgcgcccacgtctcttcgtccggcccgtcttcaagttctacgaccccggcaagacggtgccgaacacgcatgaaacttggaaaaagcccgactgccgctacctggttggaatcgaccgaggcatcaattacgttctgcgagccgtcgtcgtcgatactgaagagaagaaggttatcgccgatatcggcttgccgggcaggaagcacgaatggaggatgatccgtgacgagatcgcctaccaccaacagatgcgtgatcttgcccgcaacactggcaaacacgcgagcgtcgtggccaagcacgtccgcgccctcgcgctcgcgcgcaagaaggaccgcgcgctcggcaagttcgcaacagtcgaagccgtcgcagaacttgtcaagaagtgtgaacaggactatggtagcggcaactactgtttcgtgctcgaagacctcgacatgggggcgatgaatctcaagcgaaacaacagagtcaaacacatggcggtcatggaggaggccctcgtcaatcaaatgcgcaagcagggctatgcctatgacgggcgtcgcggtcgggtggacggcgtgaggcacgagggcgcttggtacacgagccaggtctcgccctttggctggtgggccaagcgcgacgaagtcgaggaggcgtggaagagggacaagactcgccccatcgggcgcaaggtcggcaactggtacgagatgcccgagccaggccaagacggagaccggcccgacacgtatcggaagggctactggtcgaaaccgaagaacgcggagggcaagccgtatgggcgcaaccgcttcagcgtcgagcctggcgacgagaagccggacgctgagcggcgcttctgctggggcagcgagctgttctgggatccgaacgtgaagtccttcaagggcaaggagtttcccgagggcgtcgtgctggacgccgacttcgtaggagccctcaacatcgctctccgcccgttggtcaacgacggccagggtaaaggcttcaaggccgaggacatggcgagggagcacacgatactaaacccgcagttcaagatcgcctgccagataccagtttacgagttcgtcgaagaggacggcgacaagtgggcagctctgcgccggatcatgctatag

SEQ ID NO: 12 - DNA, 2604 bp, Armatimonadetes bacterium
atggcgaaggcaaccaaagaagtcaagtcgaagcgcgtggaagcgttgcggcaggtggcgtatcaacggctggaacgcctcgagcggaaggctcagaagatcggagcgcatctgcgcaagccgggaaaagccgctgacctccaatcactccattatcttcttcacaaggtcgaagtcgaatatcacgatatcgcaaggaacctggagaaggacccgacttggacaccaaaaccgaaaatgcgacgagagaagcgtgccatcgtgccggagtccggcccggctgcgcccctcccgaccacggcaaagggtgagccgggtagaccggcaaaccgtcatattccgccaccagtgccgctcgattcagcaaggatccccgaagaccaacagtcgatgggccaaggaagcggggggaggagttggtgttctgcgcctttcgttgaggtgaagttaccgccgactcaatggtcgaatgtccgggagaagcttctgaaattccgaattgaggacgacgccgacatcgtcaggcggtgggccgaggccaagttcggaagcatcgagacggcgcgcgatggattacgtgcgagcgcagagatcggaacgagcccggatgtctggcgttccttcatcagccgcgcgatctcgaacggcaagaaggactttgagccacttctctcgttggacgatgacgaattgaccgcggatgcaacagccgagcgcgttgtgcgtcggtggcatcagattgactgggtgggccgaatgctcgactccatcctggaaaccgtcccgtcgggggtctcgaaagacacgtttcgaagcagggtcgaatcgcgtctcaagacgtttcactcgtctgtgaacagcttcgagctcaagaagaggaaggacggtacggtcgagcgcaagcggaagcacaccaacccgcagtttccgtacttgtcaccgagcgcagtgagcatcgatcctgatgttgtgactatggaggcggtcgaactgctccagatgcagcccgaggaacgctttgcaaaggacccgaacgatgcgaatggcagaatgaggctgagggttttgcaggcggaactcggcaaagcacgacgcgaggctctgggtcggcggggcgagaaggccccgccgtggagtggccgcaaggtctttcgcggaaccacgaccaggaagagggaagcgtgcctggtttgggacaaagaggcacaagcggatggactttacttcgcgctcgtgatgtcgggcggaccaaagatcgacgacaaacggtttgtctacatggacggtcagccgctacaaagcgattggcaactgcacaacggagtggccggtaaggcaaagtcatgcagggcgatgcctctcattttgaagcatgacttcctgcggtggtaccaccgccacattaagaaccacgacgtcaatgctcccctcgaaaagcggtgcgttcacacgacgacccagttcgttttcgtggagccggacgaaaagaagggccttcagccccggctgttcatcagacccgtattcaagttctacgatccggtctatgaagtgccggatagccactcgattgacaagaagccggactgccgatatttgatcggaattgaccgaggcgttaactacccctatcgtgccgcagtatacgattgcgagacaaactccataatcgccgacaagttcgtggacggacgaaaggcagattgggagcggatacgaaatgaactcgcataccaccagcggcgacgtgacctcctgcgcaactcgcgtgcctcttccgccgcaatacagcgagagattcgagccattgcacggattcgcaagagggagcgtgggctgaacaaagtcgagacggtcgagagcatcgcgcggctcgtcgactgggcggaagagaatctcgggaagtgcaattactgcttcgttctcgaagacctttcttcaaacttgaatctggggcgaaacaacagggtcaagcacattgccgcgatcaaggaggcgctgatcaaccagatgcgcaagcgcggatatcgtttcaaaaagagcgggaaagttgacggcgtgcgagaggagtccgcgtggtacacgagtgccgttgcgccatccggttggtgggcgaagaaggaagaagtggacggggcctggaaagcggacaagacgcggccattggcgagaaagatcggcagttactattgctgcgaagaaatcgacggactccatttgcgcggcgtgctgaaggggctcggaagggcgaagcgactcgttcttcaaagcgacgacccatccgcgccgactcgcagacgagggtttggatcagagttgttctgggacccctattgcaccgaactctgcggccacgctttcccgcaaggcgtcgtactggacgcagacttcatcggcgccttcaatattgcgctgcgaccgctggtgagggaggaacttgggaagaaggcgaaggccgtggacctggccgacaggcaccagacgctcaatccgacggttgccctccgatgcggcgtaacggcgtacgagttcgtcgaagtcgggggcgatccccggggcggtctccgaaaaatcttgctcaatcccgcagaggccgtgatataa

SEQ ID NO: 13 - protein, 854 aa, Armatimonadetes bacterium
MPKKTSTVALSPRDIRLRELGEKRLQRLRQREEKIRRHLESERGRRDFQSLHFLLHKIEVERNDLYRNLYQNEGHESYVPKPGKTKHRKELSLPSTELPSPPDEKKGPRPKKSRYVIPQPVPGINLPRLINRFGKSDQKSESDQEGRFWTSAPFIEVELPMLNAHRVIKALMRFVQKDERSVVRTWAVTKFGSIEAAREVLLAGALLQREPEIMRGFLQNIDPWGSLSDEELIRDEKAWRTVKLLAQKNWVDQIAKSIKDSAPKGVDKDTLDRRLRSGLKAFHSAANSGKHTNPQFPYLTSEKPSANFESVVDSVLEFLDLEDKDRYTIAKVDDKKRHRVTALQKELGQAKPRVRLEQERSRWAGHSYLQGTITRKRQASLVWDGHRTENGLALAIPLDGMPKIDVQRYMYQDGTSLLSDRQITSKTKSEGKDCALMPLRFKHAFLRWYTKHVENHVAEAPLERRCIHNTTQFVIVDPEGKHPRLFIRPVFKFYDSNKTIQNSNAPWCKPQCRYLIGIDRGINYVLRAVVVDTEEKAVIDDIPLPGRKREWRAIRQEIAYFQRMRDLSKSAQERNRYVVALAKARRKDRSLGKTETVEAVAKLVQNCSERFGEGNYCFVLENLELGALNLKRNNRVKHLASMEEALIYQMRKRGYFYNSRSNRVDGVRWEAARYTSQVSPFGWWAKRDEVEKAKKQDKSMAIGRKIGEGYEGPQDDEIESHSTIYRQGRWMKLRNEEGKAYGRSRFVVQPEDLDPAQPRRFSWGSELFWDPYQKEFKGKSFSQGVVLDADFVGALNIALRPLVNDGKGKGFTTAMMAEAHVKLNPTFEIRCKIPVYEFIAENDNSRAALRRIVI

SEQ ID NO: 14 - protein, 867 aa, Armatimonadetes bacterium
MGKNRSSSSDLSPLERSLRKVGENRLERLRVREEKIRKHIEQHPRGKNDHQALHFLLHQIEVERNDLYRNLKDPEYVPKPAKQRRERRQINVAKPPTRPKKEKGPQPESTKYVIRPPVPGKNLPAFASKYEARDTRDDSYQDGRSWTSAPYVEVELPILGADKVIQKLMKFVQKDERSIVRDWATKTYSSIEAAREALLVGAQVSEDVSVWRGLLAETKNAQNFAALSDDQIEAAMSKEAKGADLRPRRAALLVAQRHWVDQTVKAIKESAPSGVDKDTLDRRLRAGLRGFHTAANSGKHTNPQFPYLTAEKPVVPMESVVQSVLAFLDDPDDQRYTKDKEDDKKRHRVTVLQKELGKARPRKRLELQTPKWAGRPTVKGTISKRRDAALVWDTSKEANGLCLALPIGGMPKIDVEQFIYQDGTSLLSDCQIASKTTKKGAACAVLPLKPKHDFLRWFTKHVENHNPDAPLERRCLHNTTQFVIVDPEGPRPRLFVRPVFKFYDPGKTVPNTHETWKKPDCRYLVGIDRGINYVLRAVVVDTEEKKVIADIGLPGRKHEWRMIRDEIAYHQQMRDLARNTGKHASVVAKHVRALALARKKDRALGKFATVEAVAELVKKCEQDYGSGNYCFVLEDLDMGAMNLKRNNRVKHMAVMEEALVNQMRKQGYAYDGRRGRVDGVRHEGAWYTSQVSPFGWWAKRDEVEEAWKRDKTRPIGRKVGNWYEMPEPGQDGDRPDTYRKGYWSKPKNAEGKPYGRNRFSVEPGDEKPDAERRFCWGSELFWDPNVKSFKGKEFPEGVVLDADFVGALNIALRPLVNDGQGKGFKAEDMAREHTILNPQFKIACQIPVYEFVEEDGDKWAALRRIML

SEQ ID NO: 15 - protein, 867 aa, Armatimonadetes bacterium
MAKATKEVKSKRVEALRQVAYQRLERLERKAQKIGAHLRKPGKAADLQSLHYLLHKVEVEYHDIARNLEKDPTWTPKPKMRREKRAIVPESGPAAPLPTTAKGEPGRPANRHIPPPVPLDSARIPEDQQSMGQGSGGRSWCSAPFVEVKLPPTQWSNVREKLLKFRIEDDADIVRRWAEAKFGSIETARDGLRASAEIGTSPDVWRSFISRAISNGKKDFEPLLSLDDDELTADATAERVVRRWHQIDWVGRMLDSILETVPSGVSKDTFRSRVESRLKTFHSSVNSFELKKRKDGTVERKRKHTNPQFPYLSPSAVSIDPDVVTMEAVELLQMQPEERFAKDPNDANGRMRLRVLQAELGKARREALGRRGEKAPPWSGRKVFRGTTTRKREACLVWDKEAQADGLYFALVMSGGPKIDDKRFVYMDGQPLQSDWQLHNGVAGKAKSCRAMPLILKHDFLRWYHRHIKNHDVNAPLEKRCVHTTTQFVFVEPDEKKGLQPRLFIRPVFKFYDPVYEVPDSHSIDKKPDCRYLIGIDRGVNYPYRAAVYDCETNSIIADKFVDGRKADWERIRNELAYHQRRRDLLRNSRASSAAIQREIRAIARIRKRERGLNKVETVESIARLVDWAEENLGKCNYCFVLEDLSSNLNLGRNNRVKHIAAIKEALINQMRKRGYRFKKSGKVDGVREESAWYTSAVAPSGWWAKKEEVDGAWKADKTRPLARKIGSYYCCEEIDGLHLRGVLKGLGRAKRLVLQSDDPSAPTRRRGFGSELFWDPYCTELCGHAFPQGVVLDADFIGAFNIALRPLVREELGKKAKAVDLADRHQTLNPTVALRCGVTAYEFVEVGGDPRGGLRKILLNPAEAVI

SEQ ID NO: 16 - DNA, 8727 bp, Armatimonadetes bacterium
cgcatctggggctaaccctttttccgtcaccgctcacccggatcgcggaccagcgccacacgatacgcgccgcttcccgtaatcgccattaggaccgtcagcagccccttccccggaacgtttcccgcctcaaaccgcaccgtctcccccgccgcaatctcgaaggtctccgtaagcgagtaaagccgattcgtcaccttgatgcggtgctctcccggctctaacggagcttcaaacgactcgccaaacgccaacgtctcctccggccaatcgtccacccgaaggtagagatcgcgaatcccaatgtccgtcgacatcgtccgatccaccaccaacaaccccccatccccctcaccaccatcaaccatccccccattctttcaccatcggcgcccagactgagcctttcggcaatagtatcgactgctcatcgatccaatttttcagtgcctttgatccggcattgacaccgttggcccttctgtgatagtacattctccatgcccaaaaagacatcgacggtcgctctgtcacccagagatattcgcttgcgcgaacttggagagaagcgacttcaaaggttgcgacagcgcgaagagaagattcgtcgtcacctggagtcggagcgcgggcggcgtgactttcaatcgctgcactttcttcttcataaaattgaagttgaacgaaacgatctgtaccgaaatctttaccagaacgaagggcacgagtcgtatgtgccaaagccaggtaagacgaaacataggaaagaactttctttgccatccacagagttaccgtctccacctgatgagaagaaagggccccggcccaaaaagagtcgctatgtgattccccagcccgtacctggaatcaatctcccacgattgatcaatagattcggtaaatcggatcaaaagtccgaatcggatcaagaaggcagattttggacttcagcgcctttcatcgaagttgagctgcctatgcttaatgcccatcgagtcataaaggcgttgatgcgattcgttcagaaagacgagcgttcggttgtccgaacatgggctgtgaccaagtttggcagcattgaggccgccagagaagtcctgctagcaggagctttgctgcaaagagagccggaaatcatgagaggcttcctccagaatattgacccctgggggagtttgagcgatgaggaactcattcgcgatgaaaaagcgtggcggacggtgaagctcctagcccaaaagaattgggtggatcaaatcgcgaagtcgatcaaggactcggcgcctaagggcgtagataaagacactttggatcgtcgcctgcggagtggcttaaaagcattccattctgcggcaaattcaggaaaacacacgaatccccagtttccctatttgacatcggagaaaccgtccgcgaactttgaatcagttgtcgactctgtgcttgagttcctcgatctggaggacaaggatcgatacacgattgcgaaggttgacgacaagaaacgccaccgagtgacggctctgcaaaaggagctaggccaagccaaaccacgtgtaaggttggagcaggaacgtagcaggtgggctggccactcgtatctccaagggaccattaccaggaaaaggcaggcttccctcgtttgggatggtcaccgaacggagaacggtttggctctcgccatcccattagatggcatgccgaaaattgacgtgcagcgatatatgtatcaagatggcacctcccttctctcggatcggcaaattacttccaagaccaagtccgagggtaaggactgtgccttgatgcctctacgatttaagcatgcctttcttcgatggtatacgaaacacgtcgaaaatcacgtggccgaggcccctttggaacggcgatgcattcataacacaacgcagtttgtcatcgtcgacccagaaggaaagcatcctcggctgttcatccgacctgtcttcaaattctatgactctaataagacaatacagaacagtaacgccccctggtgcaaaccgcagtgtcgataccttatcggcattgatcggggcatcaactacgtgctacgagcggttgttgtagatactgaagagaaagccgtaatcgacgacatccctctaccgggtcgaaagcgggagtggcgagccattcggcaagagatcgcgtactttcagcgcatgcgggacctctccaagagtgcccaagaaaggaaccgctatgtggttgcgcttgccaaagctcgtaggaaagatcgcagcttgggcaagacagagacggttgaggctgttgcaaaacttgttcagaattgcagcgagagatttggggaaggaaactactgcttcgtgttggagaaccttgagttaggtgctcttaacttaaaaagaaataaccgagttaaacatctcgcttccatggaagaggcattgatctatcagatgcggaaaagaggctacttctacaactcccgatcgaaccgggtggatggtgttcgttgggaagccgcacgctatacaagtcaagtttctccgtttggctggtgggccaagcgcgatgaagtggagaaggccaagaaacaagataaaagcatggcgattggccgcaagattggcgagggatatgaaggtccgcaggacgatgaaatagaaagtcattcgactatctatcggcagggcagatggatgaaactcagaaatgaagaaggaaaggcctacggaagaagtcggtttgtggttcagccggaagacttggaccctgcacaacccagaaggttcagttggggaagtgaacttttctgggatccctatcaaaaggaatttaaaggaaagtccttctctcaaggcgttgtgttggatgctgattttgtgggagccctaaacattgcccttcggccactagtcaacgacggcaaaggcaaaggtttcaccaccgcgatgatggcggaagcacatgtcaagttgaacccgacctttgagatccgttgcaagatcccggtttacgaatttatcgctgagaacgacaattctcgtgccgcgctgagaaggattgtgatatagtttctaagttccatctcgatgcggaacggatactacgctgtagtctatacgacacgagtgatagccctgcggggttcgcccctaagtccgtatgacaagcctctttcaggcggtggacttctaagagtgctggtgggtggaatccctaagccacccacctccttcacaatctggcagaatcacgagttccattaagaaaaggtgacgttaacctaatttgtgcgttacgaaattgttgatggttatggtagccaggtcctcaagcatagcgagcgtctcgtacttcgcatcccttctgccctagataagaatattaagcgggaagtgcctgtcctccatctggaccatctgcttgtggggacaaagggagtgctcgtatcgagcgatgcccttgccttatgctgcgagcgaggtattcccgttacggttgtcgattggagggggcgtcccgtaggaaggttcggtagccccgcgcttcacggctccgcccatattcgccgagctcagctcgaggcctttgacgccaatttaggcgctgagttcgctcgggaagtcgtctgcgggaagctcttgaatcaagctgacaatcttcgatattttggcaaaaatcgaaagactcgcgaccccgcgcaacacgaactgcttgagacctcggcagatgatatcaatgagatttcaaaaagagcttcctgtatctctgaaaagtgcgcgaatacagcacggttgccgttgatgaccttagaagccgaaggcgctcgtatctattggtccgccctctcgcgcctctatgggacaagatccggattcgctcggcgggagcaaagaggcacgagagaccccgtaaacgccgcgctcaactacgcctatggagtactcaatggcgaggtgtggaacgccgtggtcctggctgggctagagccatacgccggattgttgcacgttgaccggcccggaagactttcgtttgtcttggacctgatggaagagtttcgcccgattatcgcggatcgcctcgtctttggtcttgcggctaagggttggaaaatcggtcaagaggagaacggatggctggattttgccaccaagcagagactgcttaaagcgatttccgagcggtgggatgcacgcgttaactatcaaggacgaaaaattcgcctgagaagtgtgcttcaacttcaagccagagatgccgctcgccacttcctcggtcgcgcccaatatcgtgcttttcgacaaaggtggtaatgagatgaagtggctcgtttgttacgatatcgaaaaggacagcgttcggcagaagattgccgacttttgtctggataaaggactcgaacgtgtccagtacagcgtcttcctgggcgatatgaatcaaacgttggcgtttgatctcgccgctcagattcgtcgtcgaatgggtgatcatccaggtcaagtccgatttattcctatctgcgaccgtgattggaaaaagacctttcggattcagataggcaactacatgggagttaagcctagcaatggtaaatgaaactcatccctacgcgcaggcggatattgttagtgtcagtgaattaaggcagtggagctattgtccccgggtcgtttggtatggccgttcaatgggcgattaccgcccaacaagcggagctatgaaagcaggcattgatgcagaggcagagcgtcaaaggttggaaactcgccgaggtttttcgcagtatgggatcaccgccgttgacaaaagattccagttttcagttcgttcggattcgcttgggcttgccggaagaatagattgcctgatcgagacaaccgatgtaacttttgaggaagcccaagcgggcctacgccccaacgaatggaattgggatgatcctctctttgtgcccgtggagtacaagacaacgttccgggttcaacaaaagcacaacgtcatgcagcttgcggcttatgcccgaatgctggagagtctaacaggaactgccgtgccgttcggattcattgtgatgctgcccgaagaagaggttcttaagattgagataagttccgagatcaagcggtcactcgacttcttaatcgaagaagttcaaagaggcttggtgtcggatgaacttcccaggcccaccccgcactcgggcaagtgtcaaaactgcgagtttcgtcgattttgcaacgacgtttggtgagaccttcgaaaccttgttgatggccgattgcatcggcagatgtgcgaatctgaactacatcgttctgcgaattgagcctcgtgagcgctcccaggtagatcagttcgcctgatgttcgagctttcctaacgtcaaattctttgattctatgctgtatcctaatatcgaaccaactgttggcttcgcacatcaaggcaccttgataggttaattgtggatttcgaaaattcgcccttctacttgttagggacaagttgggcgagagtttgtatgacgcctcagttcaaatgagaacggtcgcatgaggcgagaagcactcttagggaatgaaagcgctcttggcgtgccatgcccccgagttgtgggcggtcagtcgcatgaggcgagaagcactcttagggaatgaaaggccattcctcctcaggattcagaagctcggggaattgtcgcatgaggcgagaagcactcttagggaatgaaagttcaagaaggcatcagacgagtacttcgccgcccgtcgcatgaggcgagaagcactcttagggaatgaaagaagattcgcgctattactacgcggttctggtgctgacgtcgcatgaggcgagaagcactcttaggaaatgaaagttccatcgtccatcggtgcccgaggggtccaaacttcgtcgcatgaggcgagaagcactcttagggaatgaaagcgagatcggttcgagatcgtccttgggctttgcccgtcgcatgaggcgagaagcactcttagggaatgaaagaatcaatgatccccggacactctttaggagtgtccatgtcgcatgaggcgagaagcactcttagggaatgaaagtggaagatctgaacgtcgcgctgcttaaatggcagtgtcgcatgaggcgagaagcactcttagggaatgaaagctcttgagctttggccgcttccaggtcagtatcagtcgcatgaggcgagaagcactcttagggaatgaaagatgatcaaatgcactcgcaaggattgtgacaactggatcgtcgcatgaggcgagaagcactcttagggaatgaaagaacgagcaaccggcagtagagcttcttgagactgatgtcgcatgaggcgagaagcactcttagggaatgaaaggtgagaatgccatgacgatggacgcgcggcggtcgtcgcatgaggcgagaagcactcttagggaatgaaagaaatcgagttgtcaaggattacttgacaattgagaaagtcgcatgaggcgagaagcactcttagggaatgaaagtgtttcgcagattcaggcaggacttgcgacatcttcagcattgtcgcatgaggcgagaagcactcttagggaatgaaagagcagccgccgacccgttgcccctccggactccagcgtcgcatgaggcgagaagcactcttagggaatgaaagcccgaagaggcggtaaagagccccatctctcgcaaggtcgcatgaggcgagaagcactcttagggaatgaaaggtggtcaccgatggtggagtcaagaaaacagtcggtagtcgcatgaggcgagaagcactcttagggaatgaaagaagagagcgatgcggcctgtccaaagtagctccatccgtcgcttgaggcgagaagcactcttagggaatgaaagacctacgcttgcttggtcgccagttgtttctagtcgcatgaggcgagaagcactcttagggaatgaaagcgcgaagtcgtcttcgtggtagcggtggaacatgacgtcgcatgaggcgagaagcactcttagggaatgaaagttgtaccagtccgggcgtgtgccgatctcctgggttggtcgcatgaggcgagaagcactcttagggaatgaaagaacttttcgtccagtcgtcacgtcgatgatatcgaaaagtcgcatgaggcgagaagcactcttagggaatgaaagggaggaagcgctcccgctccgggtccttgagcgaggtcgcatgaggcgagaagcactcttagggaatgaaagattgtgccgccaggtgcatcccctggactgcttatggcgtcgcatgaggcgagaagcactcttagggaatgaaagcctcacgcaaaacgtttatgcccccggtctcgcttcctgtcgcatgaggcgagaagcactcttagggaatgaaaggctcgatcaaggccatggccaaggaactcggtgtccgtcgcatgaggcgagaagcactcttagggaatgaaagcgcacaactcgggctagaatcaagcggggatgccgagtcgcatgaggcgagaagcactcttagggaatgaaagaacttgcgacctgtggacgtgcctgcaaaaagtctgtgtcgcatgaggcgagaagcactcttagggaatgaaagttggacctcagtgtctaagttgttggacgcaagcgtagcatgaggcgagaagcactcttagggaatgaaagttatgacgcagcgagggctcacctttgatcgctacgtcgcatgaggcgagaagcactcttagggaatgaaagaagcaaaacgaagatcgttgccctcgtcgactctggtcgtcgcatgaggcgagaagcactcttagggaatgaaagtggagaggctggctacatcgttgggaacaacctgggtcgcatgaggcgagaagcactcttagggaatgaaagaacgtctgtaagtggggctgatcttgcggtgttttatgtcgcatgaggcgagaagcactcttagggaatgaaagctagtagttcgtcgatgttcatctcaactcccaggccgtcgcatgaggcgagaagcactcttagggaatgaaaggcagaacatttcgaactcggcggcgacttacatcctggtcgcatgaggcgagaagcactcttagggaatgaaagttacagatagggcaaacaaatttccagtcatagaagtcggtcgcatgaggcgagaagcactcttaggggcacctgatcttagcaactggtcgcaacaacctcgcacggcgaggttgttgttgatttgggggtaggcataacgcgtaatcgcgtagcccgggccgagtcgtcgtgccaaggcgcaattgcaaagctaagctaggccaagcaaagcaaagcggagcaaacgagcgggcccgccccgccagacccctagtctgctaaaattccaagtaatgagcttcagcgtccgactatccaccgacctgatcgcggtagaggccggcgccaccgtgcccctctccatcgaagtctcgaacaaagggaccgagacggatcgctatgagctccaaatcgagggcattgatgccgaatggacggccgtgccggaggccgtcttcgtcgtcgattcaggcgaacttcacacggaaaaggtcttctttaaggtcccccgcaccagcgagagcctggccgggaattacccctttgtcgtcaaggtgcgctcgctcaactcaggcgacgcccgtaccgctcaag

SEQ ID NO: 17 - DNA, 7476 bp, Armatimonadetes bacterium
tcgggagctgaagatcgcccgcgatccggactgcccggtgtgcggcgactcgccgacgatccgccagctcatcgactacccctcgttctgcggcttgcccggctacggctccgtcccctctcacttcgaccacgagatcgaacctgccgaactgaggtccctgctcgattcggggggcggattcgtcctgctcgacgttcgagagccccatgagttcgagatcaaccggatcgaaggctctcggttgctgcctctgggcgatctgcccgcgagggttcatgaactggattcggccgacgaaatcgtggtttattgcctgatgggctcgcgcagcgccgacgcatgcaggttcctccgcgcggcgggttttcagaaggtgcgcaacctccgcggcggcatccgctcgtggatcgatgaggtcgagccggggatggtgaagtattgagtcccgcgttcagtgcgggttgcaactcccaattggttgggggtaacaagcaaggatgggtaagaatcgatcctcgtcctcggatttgagcccgctcgaacggtccttgcggaaggtcggtgagaatcgccttgagcggctgcgggtgcgagaggagaagattaggaagcacatagaacagcacccccgcggtaagaacgatcatcaggctctccacttcttattgcaccaaatcgaggtcgagcgtaacgacctgtaccgaaacctcaaagaccccgagtacgtgcccaaaccagcgaaacagcggcgcgaaagacggcagatcaacgtcgccaaacccccgactcgaccaaagaaggaaaaggggcctcaaccagagtcgacgaagtacgtgatccgtccaccagtccctgggaaaaaccttcctgcctttgctagcaagtacgaggcgcgagacacgcgggacgattcctaccaggacggtcgctcatggacctccgcaccatatgttgaagtcgaacttcccatccttggtgcagacaaagtcatccagaaactgatgaagttcgtgcagaaggacgagcggtcgatcgtgcgcgactgggcgacaaagacgtatagctcgatcgaagccgcaagagaagcactccttgtcggggcacaagtctcggaagacgtttcggtctggcgcggactcctcgcagaaacgaagaacgcacagaacttcgccgccctctccgacgatcagatcgaagcagcgatgtcgaaggaggcgaagggcgcggacttgcgtccgaggcgcgccgcactgctggtcgcacagcgccactgggtggatcagaccgtcaaagcaatcaaggagtccgcaccgtccggcgtcgacaaggacactctcgatcgccgtctgcgcgcaggtctgagggggtttcatactgcggccaactcaggcaagcacacgaacccgcagttcccatacctcaccgcagagaagccggtagtcccgatggagtctgttgttcagagcgtattggcctttctcgacgatccagacgatcaaaggtacacgaaggacaaagaagacgacaagaagcgccaccgcgtcactgtcttgcagaaggagctcggaaaggcgaggccacgaaaacggttagaactccaaacgccgaaatgggccggcaggcccacggtaaaaggaaccatcagcaaacggcgcgacgcagcgctcgtctgggacacaagcaaagaagcgaacgggctttgtctcgcgctcccaatcgggggcatgccgaagatagacgtcgagcagttcatctaccaggatgggacgtcgctcctgtccgattgccagatcgcatcgaaaacgaccaagaagggcgcggcttgcgcagtcttgccgctcaagcccaagcatgacttcctgcgctggttcaccaagcacgtcgagaaccacaatcccgacgctccactggaacgcaggtgcctccacaacacgacccagttcgtcatagtcgacccagaagggccgcgcccacgtctcttcgtccggcccgtcttcaagttctacgaccccggcaagacggtgccgaacacgcatgaaacttggaaaaagcccgactgccgctacctggttggaatcgaccgaggcatcaattacgttctgcgagccgtcgtcgtcgatactgaagagaagaaggttatcgccgatatcggcttgccgggcaggaagcacgaatggaggatgatccgtgacgagatcgcctaccaccaacagatgcgtgatcttgcccgcaacactggcaaacacgcgagcgtcgtggccaagcacgtccgcgccctcgcgctcgcgcgcaagaaggaccgcgcgctcggcaagttcgcaacagtcgaagccgtcgcagaacttgtcaagaagtgtgaacaggactatggtagcggcaactactgtttcgtgctcgaagacctcgacatgggggcgatgaatctcaagcgaaacaacagagtcaaacacatggcggtcatggaggaggccctcgtcaatcaaatgcgcaagcagggctatgcctatgacgggcgtcgcggtcgggtggacggcgtgaggcacgagggcgcttggtacacgagccaggtctcgccctttggctggtgggccaagcgcgacgaagtcgaggaggcgtggaagagggacaagactcgccccatcgggcgcaaggtcggcaactggtacgagatgcccgagccaggccaagacggagaccggcccgacacgtatcggaagggctactggtcgaaaccgaagaacgcggagggcaagccgtatgggcgcaaccgcttcagcgtcgagcctggcgacgagaagccggacgctgagcggcgcttctgctggggcagcgagctgttctgggatccgaacgtgaagtccttcaagggcaaggagtttcccgagggcgtcgtgctggacgccgacttcgtaggagccctcaacatcgctctccgcccgttggtcaacgacggccagggtaaaggcttcaaggccgaggacatggcgagggagcacacgatactaaacccgcagttcaagatcgcctgccagataccagtttacgagttcgtcgaagaggacggcgacaagtgggcagctctgcgccggatcatgctatagttaggcgttccgtctcgactatgccgtaccactagaccgagcctacacggcacgcggtcatagcgttaaccaaggcgtggtgacaagcctctttcaggcgtcggacacttaagagcgttaggcgggcggtccctaagccgcccgcccccttatttgcacgttttccccgaaccccgtaactctgccagccacaaacacccagaagcgcgtctacactccctacgggggtagacagacatgcgctatgagatcgtagacggttacgggtgccaagtgctcaagcacagcgagcgcctcgttcttcgataccccctcgcccctccgggggagagggggccagggggtgaggggaagcaacccaagcgcgaggtgcccgtcttgcaccttgaccacttgctcatcggcacgaagggcgtgacggtctcgaccgacgccctcgctctgtgctgtgagcgcgggattcccgtcacggtcgtggactggcgcgggcggccggtgggtaggttcggaagccctgctctccacggcacagcacaggtgcgccgcgcgcagatcgctgctttcgccaccgagataggagcaaccttcgcccgagaggtcgtcagcggcaagttgctcaaccaggccgacaacctccgatacatcggcaagaacagaaagacacgggcgcccaacgagcacgaggccctgacaaggacagctgacacgctccaacgtctggcaaagaaggcagcgacggtcaagggcaagaacgcggacgacgtgcggctcccgctcatgacggtcgaggccgagggcgcgcgcgcctactggtccgttctcagcgaggtttacggcgagaggtcgggcttcgcaaagcgagagcaacgtggcacacgcgacccggtcaacgccgcgctcaactacgcctacggagtcctgaacggcgaggtctggaatgccacgatcctcgctggcctcgagccgtacgcagggttcctgcatgtggatcggccgggacgcctgagcttcgtgctcgatctgatggaagagttccgccccgtggtcgcggaccgcgtcgtgttcggactggtggcgaagggctggaagatcggccaggaggagaacggctggctggacatgccgacaaaaaggagactgattcaggcgatcggcgagaggtggggagcgcgcgtcctgcatcaagcccggaaactgcaattgcgatctgtcctccagcttcaggcacgcgacgccgcaaggcacttccagggcaaggcggagtacatcgcgttccgcctgaggtggtaggccatgaagtggctcgtgtgctacgacatcgagaaggatagtgtccgaaacaaggtggcagacttctgcctggacaaggggctcgagcgggttcaatacagcgtcttccttgggtcgatgacaagaacgctcgccaaagaacttggcgcacagatcagacggaagatgggcaagaaccccggccaggtgcggttcgtgccgatctgcgacaaagactggaagacgtcgttccgcgtccaggtcggcgaccacatgggagagaaggcgagcgatggcaagtagcgcccacgcctatcagccgagagacacggtaagcgtaagcgaactccgccagtggatgtactgcccgcgcgttgtttggtacggacgctcgatgggcgactaccgtcccacgacaggcgctatgaaagtcgggatcgaggcggaggcggagcgccaaaggctggaggagcgccggtcgttcgcgcagtacgggctggaggcatgcaacaagcgattccaagtgcctgtagcgtcggaggcgttggggctgtcggggcgcattgactgcctgatcgaacttacgcccgtttcgctggaggacgctcaggtcggggtgaggccgttgaactggaaggtgggcgatccgatgttcgtgccagtcgagtacaaatggacgtccagggctgaccagaggcagaacacaattcagctggccgcttatgggatgattctggaaagcttgacggggacgcccgtgccgctcggcttcatcgcgctcctgcctgaggaagaggttgtccgtgtcgaactcgtccctagagttaggcgcgcagtcaaatgcaccctagacgaggctcgcgaaggcctctccgccccggaactcccctggccaacgctccaccgcggaaagtgccaagactgcgagttccgacggttttgcaatgacgtctggtagatggggttcgaaaccctcgcaaccgctcggatttcggggcgatgtgcgaatcctaacgcccccgacactcaagatgatggaggccttcgacccacaatcaagcccgactcagacagcgaagagccaatggtgtgcgcaaattcgctctcaccgggtacattcattcgcacaacggcggactttgtaagctaaatagcgggtttcgaaacgacgcggccgaacctgttagggcagggttgcagcaaatggacggcgtgcttgagtccggccatgcatggtcgcaggggatcaagaacgctcttagggaatgaaagacgggcagtatcggagggcgcgggagaggtgccagccttcgtcgcaggggatcaagaacgctcttagggaatgaaagcgttgcaggcaatgatctctgcaactggccgtcgaatgtcgcaggggatcaagaacgctcttagggaatgaaagggagtgggtgcgacttaaagcgtttgacggtggctgtcgcaggggatcaagaacgctcttagggaatgaaagaaacaaagacgaattgcctggacgcaggcaaaatacgtcgcaggggatcaagaacgctcttagggaatgaaagattgcgacagcctcgcctcattccaggccgacaggctggtcgcaggggatcaagaacgctcttagggaatgaaagatcgttcctgcacgagagcggagtgagtcgccggggggtcgcaggggatcaagaacgctcttagggaatgaaaggattacctaaccacccccagcagggcttccggccctggtcgcaggggatcaagaacgctcttagggaatgaaagggagagacgggcagggagaatgaacaatgaaagcgatccgtcgcaggggatcaagaacgctcttagggaatgaaagctcgcccaaccctcggacaggttgggtggcggggtcgcaggggatcaagaacgctcttagggaatgaaagttgaacccgccaaagcagttcagccacgccgccaggtcgcaggggatcaagaacgctcttagggaatgaaagtattccacgcagcacatcggagaacttgcgtttcggtgtcgcaggggatcaagaacgctcttagggaatgaaagtgcgtagtgcgagcggatctatccgaggttgaccggagtcgcaggggatcaagaacgctcttagggaatgaaaggtatgctccaagaccttcgtcgtcgggttccaggccatgtcgcaggggatcaagaacgctcttagggaatgaaagtgtaattctgatacaggcgcgacctcacgagcacccgtcgcaggggatcaagaacgctcttagggaatgaaagcattctgcgcggcctatacatcgaatacctgcgaacgtcgcaggggatcaagaacgctcttagggaatgaaagattgccttcagccgcttagccattcgctcggcatagcaagtcgcaggggatcaagaacgctcttagggaatgaaagtttgccggccgagacgtgccaccgcctagtcaggcgatgtcgcaggggatcaagaacgctcttagggaatgaaagaggtttcgggcctggaatacccggcacccggtcggatgtcgcaggggatcaagaacgctcttagggaatgagaattgaggtaaacagagttgtggcggacttcatttgcgtcaggaggcgacgatgaggcgagagagaacggttctgtacttgttggcgggcggatttcttctgaccgcgatcgaggtgcgatatctgcaccgggaggtcttaggggagcactggcaagcttgggttcctgtggcttatggggcgctaggcacggccgtagccatcgttgcggccgcttcggccaaggcgcgaaggctggcttcgggcgtgttcgcgctcggcgtggtggcgggcctgctcggtttctaccttcacaccgagggcaaccccgcggaggtcacgaagatgttggggccggcactggtggcgcaggccgacgaagaagacgagcaccggtcttccggcgagagccatgagagttcggaggaaccgccctcgttcgcgccgctcggcctttcgggccttgccgcaatagggttcgtgtgtacctcgaagctgttcgcggccgaaaaacgttgaacct

SEQ ID NO: 18 - DNA, 5575 bp, Armatimonadetes bacterium
acgaggcagcgcgtctatttcttcagcggtctgctcgttttgatcggcgttctgtgcatgaggtggaacgtagtcatcggcggacaactgttctcaaaatcgctgcaggggttgacgacgtaccggctggaactcgtcggaggagagggagcgttggtggcgatcggcgtcctattcctcccgctcgcgatcctgttcgtcctcgcgaagatcctcccgccctggaaggcccacgaagaagcccgcgcgtagggacagatccgctcgccggctggcgcagtctagcggctagcccttgacgggccggtcgggcgaaccggctcaagcgcagcgggcgcccctgccaacccgcgcttgaggggcagtcccgccgtctcggcggtggcatgcgcgctgacaccccagctgagcgcaccaacccagggcaggagactgggaagtcatggaggagccgccgggagggccggagaaggagaagtcggatcttgaaggaacgagcgatggcgaaggcaaccaaagaagtcaagtcgaagcgcgtggaagcgttgcggcaggtggcgtatcaacggctggaacgcctcgagcggaaggctcagaagatcggagcgcatctgcgcaagccgggaaaagccgctgacctccaatcactccattatcttcttcacaaggtcgaagtcgaatatcacgatatcgcaaggaacctggagaaggacccgacttggacaccaaaaccgaaaatgcgacgagagaagcgtgccatcgtgccggagtccggcccggctgcgcccctcccgaccacggcaaagggtgagccgggtagaccggcaaaccgtcatattccgccaccagtgccgctcgattcagcaaggatccccgaagaccaacagtcgatgggccaaggaagcggggggaggagttggtgttctgcgcctttcgttgaggtgaagttaccgccgactcaatggtcgaatgtccgggagaagcttctgaaattccgaattgaggacgacgccgacatcgtcaggcggtgggccgaggccaagttcggaagcatcgagacggcgcgcgatggattacgtgcgagcgcagagatcggaacgagcccggatgtctggcgttccttcatcagccgcgcgatctcgaacggcaagaaggactttgagccacttctctcgttggacgatgacgaattgaccgcggatgcaacagccgagcgcgttgtgcgtcggtggcatcagattgactgggtgggccgaatgctcgactccatcctggaaaccgtcccgtcgggggtctcgaaagacacgtttcgaagcagggtcgaatcgcgtctcaagacgtttcactcgtctgtgaacagcttcgagctcaagaagaggaaggacggtacggtcgagcgcaagcggaagcacaccaacccgcagtttccgtacttgtcaccgagcgcagtgagcatcgatcctgatgttgtgactatggaggcggtcgaactgctccagatgcagcccgaggaacgctttgcaaaggacccgaacgatgcgaatggcagaatgaggctgagggttttgcaggcggaactcggcaaagcacgacgcgaggctctgggtcggcggggcgagaaggccccgccgtggagtggccgcaaggtctttcgcggaaccacgaccaggaagagggaagcgtgcctggtttgggacaaagaggcacaagcggatggactttacttcgcgctcgtgatgtcgggcggaccaaagatcgacgacaaacggtttgtctacatggacggtcagccgctacaaagcgattggcaactgcacaacggagtggccggtaaggcaaagtcatgcagggcgatgcctctcattttgaagcatgacttcctgcggtggtaccaccgccacattaagaaccacgacgtcaatgctcccctcgaaaagcggtgcgttcacacgacgacccagttcgttttcgtggagccggacgaaaagaagggccttcagccccggctgttcatcagacccgtattcaagttctacgatccggtctatgaagtgccggatagccactcgattgacaagaagccggactgccgatatttgatcggaattgaccgaggcgttaactacccctatcgtgccgcagtatacgattgcgagacaaactccataatcgccgacaagttcgtggacggacgaaaggcagattgggagcggatacgaaatgaactcgcataccaccagcggcgacgtgacctcctgcgcaactcgcgtgcctcttccgccgcaatacagcgagagattcgagccattgcacggattcgcaagagggagcgtgggctgaacaaagtcgagacggtcgagagcatcgcgcggctcgtcgactgggcggaagagaatctcgggaagtgcaattactgcttcgttctcgaagacctttcttcaaacttgaatctggggcgaaacaacagggtcaagcacattgccgcgatcaaggaggcgctgatcaaccagatgcgcaagcgcggatatcgtttcaaaaagagcgggaaagttgacggcgtgcgagaggagtccgcgtggtacacgagtgccgttgcgccatccggttggtgggcgaagaaggaagaagtggacggggcctggaaagcggacaagacgcggccattggcgagaaagatcggcagttactattgctgcgaagaaatcgacggactccatttgcgcggcgtgctgaaggggctcggaagggcgaagcgactcgttcttcaaagcgacgacccatccgcgccgactcgcagacgagggtttggatcagagttgttctgggacccctattgcaccgaactctgcggccacgctttcccgcaaggcgtcgtactggacgcagacttcatcggcgccttcaatattgcgctgcgaccgctggtgagggaggaacttgggaagaaggcgaaggccgtggacctggccgacaggcaccagacgctcaatccgacggttgccctccgatgcggcgtaacggcgtacgagttcgtcgaagtcgggggcgatccccggggcggtctccgaaaaatcttgctcaatcccgcagaggccgtgatataatttgaatgtgctctgccgaagacgccgcacggagcctgggccggaatcgtagatcgaacgcggcatcgaagccctgcagcccttcggggccaaggcggcgcagcaagcctctttcaggcggcagagtcctttagagtgtaacgagggcccccaggaacgggggcccagccatctccagggaagggacagaggaggtggatagtgaagtacgaatacgtcgagacattcgggtcggcggttcaaaagcactccgagcgacttgtcgtgtcggagccttccggcgaaggagggcagagaacaaagaggcaggtgcccgctctacacctggaccacctgctgatcggctcacgcggcgtcagcatttcgtcggacgctctcgaactctgctgcgaacgaggcattcccgtcacaatcgtggatcgccgcggcaagccggtggggaagttcaccgccccggcaattcacggcaccagccggacgcgccgagcgcagatcagagcgtacgagaacggcctcggggttactttcgctcgctcggtcgtcattggaaaggcggcgaatcaggcaataaacctgaaatacttcgcaaagaatcgacgagagcgaagcccggatcagtacgagacgctcagaaaatccgcagaggcgatcgaccgcgttgctcgtagagcgaagaagatctcggcgaactgcattgatgaagttcgacaaccgctcatggtccttgaggccgaggcttcgcgcatctactggagttcgttgtcagctctttacggcagcagctctggctttgtgcaccgcgagcaaaggggtaccaagaatccagtcaatgctgcgctgaactacgcctacggtgtactgacaggcgaagtttggacagcgtgcctcctggctgggcttgaaccgtacgcaggattcctacacgcggaccgaccagggaggctcagtttcgtgttggaccttatcgaggagtttcggccagtggtcgcggatagggtcgtattcgcactcgcggcgaaggggtggaggattgaacaagaggagaatggatggctctcgctcgcgtcgaaaaacaagctcctcgcgagtttggccgagaggttggattctcccgagcctgaccgcgggaggaggcgcaaactgcgcaacgtaattcagcggcaggcatacgcagcggcacagcatttcttaggaaatgaaacctacgtgccatataagcagaggtggtagcagaatgacctggctcgttgtgtatgacattgaggatgacagagttcgaacgaaggtcgcagactattgcctggacaagggtctggagcggatccaatacagttgctttcttggcgagatgtcgcgaacattggctcgcgagctggcatcaaagtgcaagcggaagctcggggacaagcccgggaagattcggcttgttcccgtttgtgaaaaggaccttgcaagccaggttcgaatcgagaatgtgccttgatcatggaagtctcgccgagtgatggattcgtctccgtatccgaggtcagacagtggtcatattgtccgcgtgtcgtctggcacaaccgctggctaggggaacgcagaccccagacgtctcgaatggaagaagggagggccgaccaggcggaacgggagcggaaggagaagaggcgcacgttcgccgaataccggttgcctgcccaatcgcgaagattcaacgtgtacttgaggtcggagcggctcggtgtttctggtgtcgtggacgccgtgctggaacttacgaatcgctccatcgacgaagtggactcccaagggctcgatccggaacgcccttatttcgcgccagttgagtataagagcacgcaagagagggtcggccgccatcatcttctgcagctcgcagggtatgcggcgctgctctccgatattacgggaacgagcgtaccgttcggatacttcgtttcgcttcccaacgggcgagcaagcagggtcgaactgagcgagaaggcgagggaagagttcctttcgtgcgtgcaagggatacgtaacatggtagtcgagtgccgaatgccggagcccacgccttcgcgggcgaagtgccgagactgtgagttccgccgcttctgcaatgacgtgtggtgagcccgcaaccctgtcaaccgacggattcccggcccaatgtgcgaaagtagcgcgggcgccgttgctcgatcctccatgccgggagtgcgaagaggtgcctccattttggggcagtaggaggcttatcggcctcctccgccgtccgcagcaggccggagccgccgaactgttcgcacattggcgcagatacactggcagatatggggttgcgaatctcgcggacgaatcgggtgtttgaaccaaaaatgcggcgataatgtacggaagcccgagtgcggagccccagcctttgaggctggcccctacgggcgcaggacaaaatgcacactctaaaggaatgaaag

SEQ ID NO: 19 - DNA, 37 bp, Armatimonadetes bacterium
gtcgcatgaggcgagaagcactcttagggaatgaaag

SEQ ID NO: 20 - DNA, 37 bp, Armatimonadetes bacterium
gtcgcaggggatcaagaacgctcttagggaatgaaag

SEQ ID NO: 21 - DNA, 37 bp, Armatimonadetes bacterium
ggcgcaggacaaaatgcacactctaaaggaatgaaag

SEQ ID NO: 22 - RNA, 74 nt, Armatimonadetes bacterium
  misc_feature (38)..(74): n is a, c, g, or u
gucgcaugaggcgagaagcacucuuagggaaugaaagnnnnnnnnnnnnnnnnnnnnnnnnnnnnnnnnnnnnn

SEQ ID NO: 23 - RNA, 74 nt, Armatimonadetes bacterium
  misc_feature (38)..(74): n is a, c, g, or u
gucgcaggggaucaagaacgcucuuagggaaugaaagnnnnnnnnnnnnnnnnnnnnnnnnnnnnnnnnnnnnn

SEQ ID NO: 24 - RNA, 74 nt, Armatimonadetes bacterium
  misc_feature (38)..(74): n is a, c, g, or u
ggcgcaggacaaaaugcacacucuaaaggaaugaaagnnnnnnnnnnnnnnnnnnnnnnnnnnnnnnnnnnnnn

SEQ ID NO: 25 - RNA, 164 nt, Armatimonadetes bacterium
uuucuaaguuccaucucgaugcggaacggauacuacgcuguagucuauacgacacgagugauagcccugcgggguucgccccuaaguccguaugacaagccucuuucaggcgguggacuucuaagagugcugguggguggaaucccuaagccacccaccuccuu

SEQ ID NO: 26 - RNA, 131 nt, Armatimonadetes bacterium
uuucuaaguuccaucucgaugcggaacggauacuacgcuguagucuauacgacacgagugauagcccugcgggguucgccccuaaguccguaugacaagccucuuucaggcgguggacuucuaagagugcu

SEQ ID NO: 27 - RNA, 151 nt, Armatimonadetes bacterium
uuaggcguuccgucucgacuaugccguaccacuagaccgagccuacacggcacgcggucauagcguuaaccaaggcguggugacaagccucuuucaggcgucggacacuuaagagcguuaggcgggcggucccuaagccgcccgcccccuu

SEQ ID NO: 28 - RNA, 119 nt, Armatimonadetes bacterium
uuaggcguuccgucucgacuaugccguaccacuagaccgagccuacacggcacgcggucauagcguuaaccaaggcguggugacaagccucuuucaggcgucggacacuuaagagcguu

SEQ ID NO: 29 - RNA, 178 nt, Armatimonadetes bacterium
uuugaaugugcucugccgaagacgccgcacggagccugggccggaaucguagaucgaacgcggcaucgaagcccugcagcccuucggggccaaggcggcgcagcaagccucuuucaggcggcagaguccuuuagaguguaacgagggcccccaggaacgggggcccagccaucuccag

SEQ ID NO: 30 - RNA, 139 nt, Armatimonadetes bacterium
uuugaaugugcucugccgaagacgccgcacggagccugggccggaaucguagaucgaacgcggcaucgaagcccugcagcccuucggggccaaggcggcgcagcaagccucuuucaggcggcagaguccuuuagagugu

SEQ ID NO: 31 - RNA, 242 nt, Artificial
  Cas-beta 1 sgRNA with terminator-like hairpin
  misc_feature (206)..(242): n is a, c, g, or u
uuucuaaguuccaucucgaugcggaacggauacuacgcuguagucuauacgacacgagugauagcccugcgggguucgccccuaaguccguaugacaagccucuuucaggcgguggacuucuaagagugcugguggguggaaucccuaagccacccaccuccuugaaagucgcaugaggcgagaagcacucuuagggaaugaaagnnnnnnnnnnnnnnnnnnnnnnnnnnnnnnnnnnnnn

SEQ ID NO: 32 - RNA, 193 nt, Artificial
  Cas-beta 1 sgRNA without terminator-like hairpin
  misc_feature (157)..(193): n is a, c, g, or u
uuucuaaguuccaucucgaugcggaacggauacuacgcuguagucuauacgacacgagugauagcccugcgggguucgccccuaaguccguaugacaagccucuuucaggcgguggacuucuaagagugcugaaaagcacucuuagggaaugaaagnnnnnnnnnnnnnnnnnnnnnnnnnnnnnnnnnnnnn

SEQ ID NO: 33 - RNA, 229 nt, Artificial
  Cas-beta 2 sgRNA with terminator-like hairpin
  misc_feature (193)..(229): n is a, c, g, or u
uuaggcguuccgucucgacuaugccguaccacuagaccgagccuacacggcacgcggucauagcguuaaccaaggcguggugacaagccucuuucaggcgucggacacuuaagagcguuaggcgggcggucccuaagccgcccgcccccuugaaagucgcaggggaucaagaacgcucuuagggaaugaaagnnnnnnnnnnnnnnnnnnnnnnnnnnnnnnnnnnnnn

SEQ ID NO: 34 - RNA, 181 nt, Artificial
  Cas-beta 2 sgRNA without terminator-like hairpin
  misc_feature (145)..(181): n is a, c, g, or u
uuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaagnnnnnnnnnnnnnnnnnnnnnnnnnnnnnnnnnnnnn

SEQ ID NO: 35 - RNA, 256 nt, Artificial
  Cas-beta 3 sgRNA with terminator-like hairpin
  misc_feature (220)..(256): n is a, c, g, or u
uuugaaugugcucugccgaagacgccgcacggagccugggccggaaucguagaucgaacgcggcaucgaagcccugcagcccuucggggccaaggcggcgcagcaagccucuuucaggcggcagaguccuuuagaguguaacgagggcccccaggaacgggggcccagccaucuccaggaaaggcgcaggacaaaaugcacacucuaaaggaaugaaagnnnnnnnnnnnnnnnnnnnnnnnnnnnnnnnnnnnnn

SEQ ID NO: 36 - RNA, 200 nt, Artificial
  Cas-beta 3 sgRNA without terminator-like hairpin
  misc_feature (164)..(200): n is a, c, g, or u
uuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaagnnnnnnnnnnnnnnnnnnnnnnnnnnnnnnnnnnnnn

SEQ ID NO: 37 - DNA, 37 bp, Artificial
  T2 PAM library target sequence
agttgacccaacgtcgccggcgtgcacaatctagatg

SEQ ID NO: 38 - protein, 6 aa, Artificial
  6X histidine tag
HHHHHH

SEQ ID NO: 39 - protein, 10 aa, Artificial
  10X histidine tag
HHHHHHHHHH

SEQ ID NO: 40 - protein, 366 aa, Escherichia coli
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQT

SEQ ID NO: 41 - protein, 96 aa, Brachypodium distachyon
SAAGGEEDKKPAGGEGGGAHINLKVKGQDGNEVFFRIKRSTQLKKLMNAYCDRQSVDMTAIAFLFDGRRLRAEQTPDELEMEDGDEIDAMLHQTGG

SEQ ID NO: 42 - DNA, 31 bp, Artificial
  A1 oligonucleotide
cggcattcctgctgaaccgctcttccgatct

SEQ ID NO: 43 - DNA, 30 bp, Artificial
  A2 oligonucleotide
gatcggaagagcggttcagcaggaatgccg

SEQ ID NO: 44 - DNA, 22 bp, Artificial
  R0 oligonucleotide
gccagggttttcccagtcacga

SEQ ID NO: 45 - DNA, 28 bp, Artificial
  C0 oligonucleotide
gaaattctaaacgctaaagaggaagagg

SEQ ID NO: 46 - DNA, 56 bp, Artificial
  F1 oligonucleotide
ctacactctttccctacacgacgctcttccgatctaaggcggcattcctgctgaac

SEQ ID NO: 47 - DNA, 49 bp, Artificial
  R1 oligonucleotide
caagcagaagacggcatacgagctcttccgatctcggcgacgttgggtc

SEQ ID NO: 48 - DNA, 35 bp, Artificial
  Bridge amplification portion of F1 oligonucleotide
ctacactctttccctacacgacgctcttccgatct

SEQ ID NO: 49 - DNA, 34 bp, Artificial
  Bridge amplification portion of R1 oligonucleotide
caagcagaagacggcatacgagctcttccgatct

SEQ ID NO: 50 - DNA, 43 bp, Artificial
  F2 oligonucleotide
aatgatacggcgaccaccgagatctacactctttccctacacg

SEQ ID NO: 51 - DNA, 18 bp, Artificial
  R2 oligonucleotide
caagcagaagacggcata

SEQ ID NO: 52 - DNA, 60 bp, Artificial
  C1 oligonucleotide
ctacactctttccctacacgacgctcttccgatctggaataaacgctaaagaggaagagg

SEQ ID NO: 53 - DNA, 36 bp, Artificial
  Sequence resulting from cleavage and adapter ligation at position 23 of the target
gctcttccgatctacgccggcgacgttgggtcaact

SEQ ID NO: 54 - DNA, 13 bp, Artificial
  Adapter portion of SEQ ID NO. 53
gctcttccgatct

SEQ ID NO: 55 - DNA, 23 bp, Artificial
  Target portion of SEQ ID NO. 53
acgccggcgacgttgggtcaact

SEQ ID NO: 56 - DNA, 10 bp, Artificial
  Sequence 5′ of PAM
tgtcctcttc

SEQ ID NO: 57 - protein, 860 aa, Armatimonadetes bacterium
MGKNRSSSSDLSQLERSLRKVGENRLERLRVRGQKIRKHLEQHPRGKNDHQALHFLLHQIEVERNDLYRNLKDPEYVPKPAKRRRERRQINVAQPPTRPTKSVGPKPAPTTYVIPRPEPGRDLPAFASRYKASDSRGEDDQDGRSWTAAPFVEVELPIQIAGKILEKLRKYVQKDEREIVREWAVKTYGSIEAAREPLLIGAQVSEDVSVWRGLLAETKNAQDFAALSDDQIEAAMSKEAKGSDLRPRRAALLVAQRHWVDQTVKAIKESAPKGVDKDTLDRRLRAGLRGFHTAANSGKHTNPQFPYLTPKEAKVPLESVVNQVLEFLDDADDQRYVQVHRVSHLQKELGKARPRKRLELQRPKWAGRPTVQGTISKRRDAALVWDTSKKENGLCLALPLGGLQKIDVERFIYQDGTSLLSDCQIASKTSKKGAACALMPLKPKHDFLRWYTKHVENHNADAPLERRCLHNTTQFVIVDPEGQRPRLFIRPVFKFYDPGKAVPNTHETWKKPDCRYLVGIDRGINYVLRAVVVDIEKKEVIADIHLQGDKHKWRMIRDEIAYHQQMRDLASNTGKHPSVVARHVRALALARKKDRALGRFTTVKAVADIVMQCENDYGSGNYCFVLEDLDMGKMNLKRNNRVKHMAVMKEALVNQMRKRGYAYDGRRGRADGVRYEGAWYTSQVSPFGWWAKREEVEEAWKKDTSRPIGRKVGNWYEMPDPNEEGKRSDVYRKGCWKKPQNASGKPYGRNRFCVEPGDEKPDAQRRFSWGSELFWDPNVKSFKGKEFPEGVVLDADFVGALNIALRPLVNDGQGRGFTADKMAEAHTRLNPQFEIVCKIPVYEFIEEHGDKRAKLRRIVL

SEQ ID NO: 58 - protein, 21 aa, Artificial
  14X histidine tag
HHHHSGHHHTGHHHHSGSHHH

SEQ ID NO: 59 - RNA, 24 nt, Artificial
  24 nt spacer sequence (targeting T2)
aguugacccaacgucgccggcgug

SEQ ID NO: 60 - RNA, 217 nt, Artificial
  Cas-beta 2 sgRNA with terminator-like hairpin targeting T2 sequence
guuaggcguuccgucucgacuaugccguaccacuagaccgagccuacacggcacgcggucauagcguuaaccaaggcguggugacaagccucuuucaggcgucggacacuuaagagcguuaggcgggcggucccuaagccgcccgcccccuugaaagucgcaggggaucaagaacgcucuuagggaaugaaagaguugacccaacgucgccggcgug

SEQ ID NO: 61 - RNA, 169 nt, Artificial
  Cas-beta 2 sgRNA without terminator-like hairpin targeting T2 sequence
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaagaguugacccaacgucgccggcgug

SEQ ID NO: 62 - RNA, 244 nt, Artificial
  Cas-beta 3 sgRNA with terminator-like hairpin targeting T2 sequence
guuugaaugugcucugccgaagacgccgcacggagccugggccggaaucguagaucgaacgcggcaucgaagcccugcagcccuucggggccaaggcggcgcagcaagccucuuucaggcggcagaguccuuuagaguguaacgagggcccccaggaacgggggcccagccaucuccaggaaaggcgcaggacaaaaugcacacucuaaaggaaugaaagaguugacccaacgucgccggcgug

SEQ ID NO: 63 - RNA, 188 nt, Artificial
  Cas-beta 3 sgRNA without terminator-like hairpin targeting T2 sequence
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaagaguugacccaacgucgccggcgug

SEQ ID NO: 64 - DNA, 24 bp, Artificial
  T1 target sequence
tgtcctcttcctctttagcgttta

SEQ ID NO: 65 - RNA, 169 nt, Artificial
  Cas-beta 2 sgRNA without terminator-like hairpin targeting T1 sequence
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaguguccucuuccucuuuagcguuua

SEQ ID NO: 66 - RNA, 165 nt, Artificial
  Cas-beta 2 sgRNA without terminator-like hairpin targeting 20 nt of T2 sequence
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaagaguugacccaacgucgccgg

SEQ ID NO: 67 - DNA, 45 bp, Artificial
  ssDNA oligonucleotide activator
agcttcatctagattgtgcacgccggcgacgttgggtcaactggg

SEQ ID NO: 68 - DNA, 45 bp, Artificial
  Complementary oligonucleotide used to generate dsDNA activator and used as ssDNA non-specific activator
aattcccagttgacccaacgtcgccggcgtgcacaatctagatga

SEQ ID NO: 69 - DNA, 45 bp, Artificial
  Oligonucleotide 1 used to generate dsDNA non-specific activator
agctttgtcctcttcctctttagcgtttagaatttgtcgacgggg

SEQ ID NO: 70 - DNA, 45 bp, Artificial
  Oligonucleotide 2 used to generated dsDNA non-specific activator
aattccccgtcgacaaattctaaacgctaaagaggaagaggacaa

SEQ ID NO: 71 - protein, 7 aa, Simian virus 40
PKKKRKV

SEQ ID NO: 72 - DNA, 896 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 73 - DNA, 82 bp, Zea mays
tccccaacctcgtgttgttcggagcgcacacacacacaaccagatctcccccaaatccacccgtcggcacctccgcttcaag

SEQ ID NO: 74 - DNA, 1013 bp, Zea mays
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 75 - DNA, 21 bp, Artificial
  Sequence encoding SV40 NLS
ccaaagaagaagcgcaaggtc

SEQ ID NO: 76 - DNA, 96 bp, Artificial
  Exon 1 of maize optimized Cas-beta 1 nuclease
atgcctaaaaagacctcgacggtcgccctcagccccagggacatcaggctcagggagctgggcgagaagaggctgcagaggctgaggcagagggag

SEQ ID NO: 77 - DNA, 2466 bp, Artificial
  Exon 2 of maize optimized Cas-beta 1 nuclease
gagaagatccgccgccacctggagagcgagaggggccgccgcgacttccagagcctccactttctgctccataaaattgaggtcgagcgcaacgacctctacaggaacctctaccagaacgagggccacgagagctacgtccccaagcccggcaagaccaagcacaggaaggagctcagcctcccgagcaccgagctccccagcccccccgacgagaagaagggaccccgcccgaagaagagcaggtacgtcatcccacagcccgtccccggaatcaatctccccaggctcatcaacaggttcggcaagagcgaccagaagagcgagagcgaccaggagggacgcttctggacgagcgcccccttcatcgaggtcgagctcccaatgctcaacgcccaccgcgtcataaaggccctcatgcgcttcgtccagaaggacgagcgcagcgtcgtccgcacctgggccgtcaccaagttcggatcgatagaggcggccagggaggtcctcctcgccggcgccctcctgcagagggagcccgagattatgcgcggcttcctccagaatatcgacccctggggatcgctgtcggacgaggagctgatccgcgatgaaaaagcctggaggaccgtcaagctcctcgcccagaagaactgggtcgaccagatagccaagtcgataaaggactctgcgcccaagggcgtcgacaaggacaccctcgacaggcgcctccgcagcggcctcaaagcattccactctgcggccaacagcggcaagcacaccaacccccagttcccatacctcacctccgagaagcccagcgccaacttcgagtccgtcgtcgacagcgtcctggagttcctcgacctggaggacaaggaccgctacaccatagcgaaggtcgacgacaagaagaggcacagggtcaccgccctccagaaggagctcggccaggccaagccccgcgtccgcctggagcaggagaggagccgctgggccggccacagctacctccagggcaccatcacccgcaagaggcaggccagcctcgtctgggacggccacaggaccgagaacggactcgccctcgccataccgctagacggcatgcccaagatcgacgtccagaggtacatgtaccaggacggaaccagcctcctcagcgaccgccagatcaccagcaagaccaagagcgagggcaaggactgcgccctcatgcccctcaggttcaagcatgccttcctcaggtggtacaccaagcacgtcgagaaccacgtcgccgaggcccccctggagcgccgctgcatccacaacaccacccagttcgtgatagtggacccagagggaaagcatcccaggctctttatcaggcccgtgttcaagttctacgacagcaacaagacaatacagaacagcaacgccccgtggtgcaagccccagtgcaggtacctcataggcatcgaccgcggcatcaactacgtcctccgcgccgtcgtcgtcgacaccgaggagaaggccgtcatcgacgacatccccctccccggccgcaagcgcgagtggagggccatcaggcaggagatcgcctacttccagaggatgagggacctcagcaagagcgcccaggagcgcaacaggtacgtcgtcgccctcgccaaggccaggaggaaggacaggagcctgggaaagaccgaaaccgtggaggccgtcgccaagctcgtccagaactgcagcgagaggttcggcgagggcaactactgcttcgtcctggagaacctggagctcggcgccctcaacctcaagaggaacaacagggtcaagcacctcgccagcatggaggaggccctcatctaccagatgaggaagaggggctacttctacaacagcaggagcaaccgcgtcgacggcgtccgctgggaggccgccaggtacaccagccaggtcagcccgttcggatggtgggcgaagagggacgaggtcgagaaggccaagaagcaggataaaagcatggccatcggccgcaagatcggcgagggctacgagggccctcaggacgatgaaatagagtcccactctacgatctacaggcagggcaggtggatgaaactcaggaacgaggaggggaaggcgtacggaaggagcaggttcgtcgtccagcccgaggacctcgacccagcccagccccggaggttcagctggggcagcgagctgttctgggacccataccagaaggagttcaagggcaagagcttcagccagggcgtggtcctggacgccgactttgtcggcgcgctgaacatagcgctgaggcccctcgtcaacgacggcaagggcaagggcttcaccaccgccatgatggccgaggcccacgtcaagctcaacccaacgttcgagatcaggtgcaagatccccgtctacgagttcatcgccgagaacgacaacagcagggccgccctcaggcgcatcgtcatc

SEQ ID NO: 78 - DNA, 189 bp, Solanum tuberosum
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 79 - DNA, 317 bp, Solanum tuberosum
agacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggc

SEQ ID NO: 80 - DNA, 1001 bp, Zea mays
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattg

SEQ ID NO: 81 - DNA, 156 bp, Artificial
  Sequence encoding Cas-beta Affinity Domain of sgRNA
tttctaagttccatctcgatgcggaacggatactacgctgtagtctatacgacacgagtgatagccctgcggggttcgcccctaagtccgtatgacaagcctctttcaggcggtggacttctaagagtgctgaaaagcactcttagggaatgaaag

SEQ ID NO: 82 - DNA, 24 bp, Zea mays
tcctagcgcagagcatcaagctcg

SEQ ID NO: 83 - DNA, 68 bp, Hepatitis delta virus
ggccggcatggtcccagcctcctcgctggcgccggctgggcaacatgcttcggcatggcgaatgggac

SEQ ID NO: 84 - DNA, 8 bp, Zea mays
tttttttt

SEQ ID NO: 85 - protein, 92 aa, Armatimonadetes bacterium
MKWLVCYDIEKDSVRNKVADFCLDKGLERVQYSVFLGSMTRTLAKELGAQIRKRMGKNPGQVRFVPICEKDWRSSFRVQVGDHMGEKSSDDK

SEQ ID NO: 86 - protein, 221 aa, Armatimonadetes bacterium
MISRFHAYQPLDMVNVSDLRQWVYCPRVVWYGRSLGDYRPRTGAMKVGIEAEAERQRLEERRTFAQYGLGACTKRFQVPVVSEALGLSGRIDCLFELTPVALEDAQVGVRPLNWKEGDPMFAPVEYKWTSRADQRRNTIQLAAYGMILESLTGTPVPLGFIALLPEEEVVRVELGPRVRRAVKRTLDEAREGLSASELPWPTLHRGKCQDCEFRRFCNDVW

SEQ ID NO: 87 - DNA, 2583 bp, Armatimonadetes bacterium
atgggtaagaatcggtcctcgtcctcggatttgagccagctcgaacgatccttacggaaagtcggtgagaatcgccttgagcggctgcgggtgcgtgggcagaagattaggaagcaccttgaacagcacccccgaggtaagaacgatcatcaggccctccactttctgctccaccagatcgaggtcgaacggaatgacctgtaccgaaacctcaaagaccccgaatacgtgcccaagccagcgaaacggcggcgagaaagacggcagatcaacgtcgcccaaccgccgacccgacccaccaagagtgtggggccgaaaccagcgccgacgacatacgtgatcccgcgccccgagccaggccgtgacctaccagcattcgcgagcaggtacaaggcaagtgactcgagaggcgaggacgaccaagacggtcggtcatggactgccgcgccctttgtcgaagtcgagctgccgatacaaattgccggcaagatcctcgagaaactccgtaagtacgtgcaaaaggacgaacgggagatcgttcgcgagtgggctgtcaagacctatggctcgatcgaagccgcaagagaaccacttcttatcggggcacaagtctcggaagacgtctcggtctggcgcggactcctcgcagaaacgaaaaacgcacaggacttcgccgccctctccgacgatcagatcgaagcagcgatgtcgaaggaggcgaaggggtcagacctgcgtccgaggcgcgccgcactgctagtcgcacagcgccactgggtggatcagaccgtcaaggcaatcaaggagtcggccccgaaaggcgtcgataaggacacactcgatcgccgtttgcgcgctggcctaagggggtttcatacagcagctaattcgggtaagcacacgaacccacagttcccatacttgacgccgaaagaggcaaaggtgccgttagaatcggtcgtcaatcaggtcttagagttcctcgacgacgcggacgaccagcgctacgtccaggtccacagagtcagtcatctccagaaggaactcgggaaggcgaggccgcgcaagcgactggagcttcagaggccaaagtgggcgggtaggcctacagtgcaaggaacgatcagcaaacggcgcgacgccgcactcgtgtgggacacgagcaagaaggaaaacggcctctgcctcgcgctcccgctcgggggtttgcagaagatagatgttgagcggttcatctaccaagacggcacgtcactattgtcggactgccagatcgcgtcgaagacctccaagaaaggtgcggcgtgcgcgctcatgccgctcaagcccaagcacgacttcctgcgttggtacaccaaacacgtcgagaaccacaacgcagacgcgccgctcgagcgccgctgtctgcacaacacgacccagttcgtgatcgtggatccagaggggcagcgcccgcgtctcttcatccgccccgtcttcaagttctacgaccccggcaaggcagtgccgaacacgcacgaaacttggaagaagccggactgccgctacctggtagggatcgaccgaggtatcaactacgttctgcgcgctgtcgttgtggacatcgaaaagaaggaagtcatcgctgacatccacctacaaggcgacaagcacaaatggaggatgatccgcgacgagatcgcctaccaccaacagatgcgtgatcttgccagcaacacaggcaaacacccgagcgtcgtggcgaggcacgtccgcgcactcgccctcgcccgcaagaaggatcgcgcgctcggcaggtttacgacggtcaaggctgtcgcagatatcgtcatgcaatgcgaaaacgactacggaagcggtaactactgcttcgtgctcgaagacctcgacatgggcaagatgaatctcaagcgcaacaaccgcgtgaagcacatggccgtcatgaaggaagcgcttgtcaatcaaatgcgcaagcgcggctatgcctacgacggtcgccgcggccgggcggacggcgtcaggtacgagggcgcatggtacacgagccaagtgtcccccttcggctggtgggccaagcgtgaagaggtggaggaggcgtggaagaaggacacgtcgcgcccgatcggtcgcaaggtcggcaactggtacgagatgccagatccgaacgaagaaggaaagcggtcagacgtgtatcggaagggctgctggaagaaaccgcagaacgcaagcggaaagccatacgggcggaaccgcttctgtgtggaacctggcgacgagaagccggacgctcagcggcgcttctcctgggggagcgagctgttctgggacccgaacgtgaagtccttcaagggcaaagagtttcccgagggggtcgtgctggacgccgacttcgtaggagcgctcaacatcgcccttcgcccactcgtcaacgacggtcagggcaggggcttcacggcagacaagatggccgaagcgcatacgagactcaacccgcagttcgagatcgtttgcaaaatccccgtttatgagttcatcgaagagcacggtgacaagagggcaaaactcaggcggatcgtgctatag

SEQ ID NO: 88 - DNA, 5309 bp, Armatimonadetes bacterium
  misc_feature (2994)..(3091): n is a, c, g, or t
atgggtaagaatcggtcctcgtcctcggatttgagccagctcgaacgatccttacggaaagtcggtgagaatcgccttgagcggctgcgggtgcgtgggcagaagattaggaagcaccttgaacagcacccccgaggtaagaacgatcatcaggccctccactttctgctccaccagatcgaggtcgaacggaatgacctgtaccgaaacctcaaagaccccgaatacgtgcccaagccagcgaaacggcggcgagaaagacggcagatcaacgtcgcccaaccgccgacccgacccaccaagagtgtggggccgaaaccagcgccgacgacatacgtgatcccgcgccccgagccaggccgtgacctaccagcattcgcgagcaggtacaaggcaagtgactcgagaggcgaggacgaccaagacggtcggtcatggactgccgcgccctttgtcgaagtcgagctgccgatacaaattgccggcaagatcctcgagaaactccgtaagtacgtgcaaaaggacgaacgggagatcgttcgcgagtgggctgtcaagacctatggctcgatcgaagccgcaagagaaccacttcttatcggggcacaagtctcggaagacgtctcggtctggcgcggactcctcgcagaaacgaaaaacgcacaggacttcgccgccctctccgacgatcagatcgaagcagcgatgtcgaaggaggcgaaggggtcagacctgcgtccgaggcgcgccgcactgctagtcgcacagcgccactgggtggatcagaccgtcaaggcaatcaaggagtcggccccgaaaggcgtcgataaggacacactcgatcgccgtttgcgcgctggcctaagggggtttcatacagcagctaattcgggtaagcacacgaacccacagttcccatacttgacgccgaaagaggcaaaggtgccgttagaatcggtcgtcaatcaggtcttagagttcctcgacgacgcggacgaccagcgctacgtccaggtccacagagtcagtcatctccagaaggaactcgggaaggcgaggccgcgcaagcgactggagcttcagaggccaaagtgggcgggtaggcctacagtgcaaggaacgatcagcaaacggcgcgacgccgcactcgtgtgggacacgagcaagaaggaaaacggcctctgcctcgcgctcccgctcgggggtttgcagaagatagatgttgagcggttcatctaccaagacggcacgtcactattgtcggactgccagatcgcgtcgaagacctccaagaaaggtgcggcgtgcgcgctcatgccgctcaagcccaagcacgacttcctgcgttggtacaccaaacacgtcgagaaccacaacgcagacgcgccgctcgagcgccgctgtctgcacaacacgacccagttcgtgatcgtggatccagaggggcagcgcccgcgtctcttcatccgccccgtcttcaagttctacgaccccggcaaggcagtgccgaacacgcacgaaacttggaagaagccggactgccgctacctggtagggatcgaccgaggtatcaactacgttctgcgcgctgtcgttgtggacatcgaaaagaaggaagtcatcgctgacatccacctacaaggcgacaagcacaaatggaggatgatccgcgacgagatcgcctaccaccaacagatgcgtgatcttgccagcaacacaggcaaacacccgagcgtcgtggcgaggcacgtccgcgcactcgccctcgcccgcaagaaggatcgcgcgctcggcaggtttacgacggtcaaggctgtcgcagatatcgtcatgcaatgcgaaaacgactacggaagcggtaactactgcttcgtgctcgaagacctcgacatgggcaagatgaatctcaagcgcaacaaccgcgtgaagcacatggccgtcatgaaggaagcgcttgtcaatcaaatgcgcaagcgcggctatgcctacgacggtcgccgcggccgggcggacggcgtcaggtacgagggcgcatggtacacgagccaagtgtcccccttcggctggtgggccaagcgtgaagaggtggaggaggcgtggaagaaggacacgtcgcgcccgatcggtcgcaaggtcggcaactggtacgagatgccagatccgaacgaagaaggaaagcggtcagacgtgtatcggaagggctgctggaagaaaccgcagaacgcaagcggaaagccatacgggcggaaccgcttctgtgtggaacctggcgacgagaagccggacgctcagcggcgcttctcctgggggagcgagctgttctgggacccgaacgtgaagtccttcaagggcaaagagtttcccgagggggtcgtgctggacgccgacttcgtaggagcgctcaacatcgcccttcgcccactcgtcaacgacggtcagggcaggggcttcacggcagacaagatggccgaagcgcatacgagactcaacccgcagttcgagatcgtttgcaaaatccccgtttatgagttcatcgaagagcacggtgacaagagggcaaaactcaggcggatcgtgctatagtaggccgttctgactcgatgcgggacggatactacactaagcctaaacggcacgagcgatagccctgcggggattccccaaagcccgtacgacaagcctctttcaggcgtcggacacttaagagcgttaggcgggcggtccctaagccgctcgcccccttatccccacggtttccaagaaccccgtaactctgccagtcacaaacacccagaagcgcgtctacacttacttgtgagtaaggagtagatagacatgcgctatgagatcgtagatggctacgggtgccaggtgcttaagcacagcgagcgcctgatcctcaagtggtccgccagagacgaccagcaacccaagcgcgaggtgccgattctgcacctcgaccacctgctcgtcggctccaagggcgtgacggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacggtcgtggactggcgtggtcgtccagttgggaggttcggaagccccgccctccacggaacggcacaggtgcgccgagcacagatagccgccttcgacacaaaacgaggagcctctttcgccagcgagatcgtcggcggcaagctcctcaaccaggcagacaatctcaactacatcggcaagaacagaaagacgcgcgctccagaagtttacgaagagttgacaaggacagccgacacgctccaacgtctggcaaagaaggcagtggcggtcaaaggcaagaatgcggacgaaattcggatgctcctcatgacggtcgaggcagagggcgcccgcgcttactggtcggtgctcagcgaagtctacggcaaagggtcggggttcgccaagcgcgaacagcgcggcacccgcgacccggtcaacgccgtgctcaactacgcctatggcgtactcaatggcgaggtctggaacgccgtcgtgctggccggactggagccctacgcagggttcctgcacgtcgatcggccgggacgcctgagcttcgtgctcgatctgatggaggagttccgccccgtcgtcgccgaccgcgttgtgtttggcctcgtcgccaagggatggaagatcggtcaggaggagaacggttggctcgatctatcgacgaagaagcggctcgtccgggcgatcggcgacaggtgggggacgcgcgtcgagcaccaaggtcgcaagttgcaactacgatctgtgctccagctgcaggcacgggacgccgcacggcacttccaggacaaggcggagtacgacgcgttccgccagaggtggtaaggcatgaagtggctcgtgtgctacgacatcgagaaggatagtgtccgaaacaaggtggcagacttctgccttgacaaggggctcgaacgggttcaatacagcgtcttccttgggtcgatgacaagaacgctcgccaaagaacttggagcacagatcaggaagcgaatgggcaagaatccagggcaagtgcgctttgtgccgatttgtgagaaggactggcgttcgtcgttccgcgtccaagtcggtgaccacatgggagaaaagtcatccgatgataagtaggttccacgcctaccaaccacttgacatggtgaacgtgagcgatctccgccagtgggtgtactgtccgcgcgtcgtctggtacgggcgctcattgggcgactaccgcccgaggaccggggcgatgaaggtggggatagaggcggaggcggagcggcagaggctggaggagcgaaggacgttcgctcaatacggtctgggggcgtgcaccaagcggttccaggttcccgttgtgtcggaggcgttggggctgtcggggcgaatcgactgcctgttcgaacttacgcccgttgcgctggaggacgctcaggtcggggtgaggccgctgaactggaaggagggcgatccgatgttcgcgccagttgagtacaaatggacgtccagagctgaccagaggcggaacacaattcagctggccgcttatgggatgattctggaaagcttgacggggacgcccgtgccgctcggcttcatcgcgctcctgcctgaggaagaggttgtccgtgtcgaactcggccccagagttaggcgcgcagtcaaacgcaccctagacgaggctcgcgaaggcctttccgcctcggaactcccctggccaacgctccaccgcggaaagtgccaagactgcgagttccgacggttttgcaatgacgtctggtagatggggttcgaaaccctcgcagctgctcggatttcggggcgatgtgcgaatcctaacgcccccgacactcaagatgatggaggccttcgacccacaatcaagcccgactcagacagcgaagagccaatggtgtgcgcaaattcgctctcaccgggtacattcattcgcacaacggcggactttgtaagctaaatagcgggtttcgaaacgacgcggccgaacctgttagggcagggttgcagcaaatggacggcgtgtctgagtccggtcatgcatggtcgcaggggatcaagaacgctcttagggaatgaaaggaccgacggccttccgaacggagaggttctcgcgtcgtcgcaggggatcaagaacgctcttagggaatgaaagatggctctcgacgaaggcctacggctggaccgtcgagatgcaggtcaaggcggcccgcgccggcctgcgc

SEQ ID NO: 89 - RNA, 160 nt, Armatimonadetes bacterium
uaggccguucugacucgaugcgggacggauacuacacuaagccuaaacggcacgagcgauagcccugcggggauuccccaaagcccguacgacaagccucuuucaggcgucggacacuuaagagcguuaggcgggcggucccuaagccgcucgcccccuu

SEQ ID NO: 90 - RNA, 128 nt, Armatimonadetes bacterium
uaggccguucugacucgaugcgggacggauacuacacuaagccuaaacggcacgagcgauagcccugcggggauuccccaaagcccguacgacaagccucuuucaggcgucggacacuuaagagcguu

SEQ ID NO: 91 - RNA, 231 nt, Artificial
  Cas-beta 4 sgRNA with terminator-like hairpin
  misc_feature (202)..(231): n is a, c, g, or u
uaggccguucugacucgaugcgggacggauacuacacuaagccuaaacggcacgagcgauagcccugcggggauuccccaaagcccguacgacaagccucuuucaggcgucggacacuuaagagcguuaggcgggcggucccuaagccgcucgcccccuugaaagucgcaggggaucaagaacgcucuuagggaaugaaagnnnnnnnnnnnnnnnnnnnnnnnnnnnnnn

SEQ ID NO: 92 - RNA, 190 nt, Artificial
  Cas-beta 4 sgRNA without terminator-like hairpin
  misc_feature (154)..(190): n is a, c, g, or u
uaggccguucugacucgaugcgggacggauacuacacuaagccuaaacggcacgagcgauagcccugcggggauuccccaaagcccguacgacaagccucuuucaggcgucggacacuuaagagcguugaaaaacgcucuuagggaaugaaagnnnnnnnnnnnnnnnnnnnnnnnnnnnnnnnnnnnnn

SEQ ID NO: 93 - DNA, 27 bp, Homo sapiens
cccacagttcgattacctttcccactc

SEQ ID NO: 94 - DNA, 27 bp, Homo sapiens
cctttcccactcactgctttctcctct

SEQ ID NO: 95 - DNA, 27 bp, Homo sapiens
cccgccttcagaagagggtgcattttc

SEQ ID NO: 96 - DNA, 27 bp, Homo sapiens
cctgaaaatgcaccctcttctgaaggc

SEQ ID NO: 97 - RNA, 169 nt, Artificial
  Cas-beta2 sgRNA targeting WTAP site 1
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaagacaguucgauuaccuuucccacuc

SEQ ID NO: 98 - RNA, 169 nt, Artificial
  Cas-beta2 sgRNA targeting WTAP site 2
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaguucccacucacugcuuucuccucu

SEQ ID NO: 99 - RNA, 169 nt, Artificial
  Cas-beta2 sgRNA targeting RUNX1 site 1
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaggccuucagaagagggugcauuuuc

SEQ ID NO: 100 - RNA, 169 nt, Artificial
  Cas-beta2 sgRNA targeting RUNX1 site 2
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaggaaaaugcacccucuucugaaggc

SEQ ID NO: 101 - RNA, 188 nt, Artificial
  Cas-beta3 sgRNA targeting WTAP site 1
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaagacaguucgauuaccuuucccacuc

SEQ ID NO: 102 - RNA, 188 nt, Artificial
  Cas-beta3 sgRNA targeting WTAP site 2
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaaguucccacucacugcuuucuccucu

SEQ ID NO: 103 - RNA, 188 nt, Artificial
  Cas-beta3 sgRNA targeting RUNX1 site 1
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaaggccuucagaagagggugcauuuuc

SEQ ID NO: 104 - RNA, 188 nt, Artificial
  Cas-beta3 sgRNA targeting RUNX1 site 2
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaaggaaaaugcacccucuucugaaggc

SEQ ID NO: 105 - DNA, 73 bp, Artificial
  5′ FAM labeled RUNX1 site 1 non-target strand
accagcaggactacagcttccccgccttcagaagagggtgcattttcagcctttttgtgggtgtacgttttgg

SEQ ID NO: 106 - DNA, 73 bp, Artificial
  5′ ROX labeled RUNX1 site 1 target strand
ccaaaacgtacacccacaaaaaggctgaaaatgcaccctcttctgaaggcggggaagctgtagtcctgctggt

SEQ ID NO: 107 - DNA, 71 bp, Artificial
  5′ FAM labeled WTAP site 1 non-target strand
accagcaggactacagcttccccacagttcgattacctttcccactccctttttgtgggtgtacgttttgg

SEQ ID NO: 108 - DNA, 71 bp, Artificial
  5′ ROX labeled WTAP site 1 target strand
ccaaaacgtacacccacaaaaagggagtgggaaaggtaatcgaactgtggggaagctgtagtcctgctggt

SEQ ID NO: 109 - DNA, 27 bp, Homo sapiens
cccactcactgctttctcctcttgagg

SEQ ID NO: 110 - DNA, 27 bp, Homo sapiens
cccgccacgttcagaatggcttggact

SEQ ID NO: 111 - DNA, 27 bp, Homo sapiens
ccctcaagaggagaaagcagtgagtgg

SEQ ID NO: 112 - DNA, 27 bp, Homo sapiens
cccacgggcagtgaaaactctctcaca

SEQ ID NO: 113 - DNA, 27 bp, Homo sapiens
cccgtgggagagtctacactttcatac

SEQ ID NO: 114 - DNA, 27 bp, Homo sapiens
ccctcttctgaaggcgggggactcaat

SEQ ID NO: 115 - DNA, 27 bp, Homo sapiens
ccccgccttcagaagagggtgcatttt

SEQ ID NO: 116 - DNA, 27 bp, Homo sapiens
cccctctagccctacatctctctttct

SEQ ID NO: 117 - DNA, 27 bp, Homo sapiens
ccctctagccctacatctctctttctt

SEQ ID NO: 118 - DNA, 27 bp, Homo sapiens
ccctacatctctctttcttctcccctc

SEQ ID NO: 119 - DNA, 31 bp, Artificial
  Non-target strand (NTS)
ccccgtcgacaaattctaaacgctaaagagg

SEQ ID NO: 120 - DNA, 11 bp, Artificial
  Sanger sequence from the reverse direction (Cas-beta2)
cctctttagcg

SEQ ID NO: 121 - DNA, 31 bp, Artificial
  Target strand (TS)
cctctttagcgtttagaatttgtcgacgggg

SEQ ID NO: 122 - DNA, 27 bp, Artificial
  Sanger sequence from the forward drection (Cas-beta2)
ccccgtcgacaaattctaaacgctaaa

SEQ ID NO: 123 - DNA, 13 bp, Artificial
  Sanger sequence from the reverse direction (Cas-beta3)
cctctttagcgtt

SEQ ID NO: 124 - DNA, 26 bp, Artificial
  Sanger sequence from the forward drection (Cas-beta3)
ccccgtcgacaaattctaaacgctaa

SEQ ID NO: 125 - DNA, 56 bp, Homo sapiens
tcattgagtcccccgccttcagaagagggtgcattttcaggaggaagcgatggctt

SEQ ID NO: 126 - DNA, 50 bp, Homo sapiens
tcattgagtcccccgccttcagaagagggtgcaggaggaagcgatggctt

SEQ ID NO: 127 - DNA, 51 bp, Homo sapiens
tcattgagtcccccgccttcagaagagggtttcaggaggaagcgatggctt

SEQ ID NO: 128 - DNA, 51 bp, Homo sapiens
tcattgagtcccccgccttcagaagagggtgctaggaggaagcgatggctt

SEQ ID NO: 129 - DNA, 50 bp, Homo sapiens
tcattgagtcccccgccttcagaagagttttcaggaggaagcgatggctt

SEQ ID NO: 130 - DNA, 51 bp, Homo sapiens
tcattgagtcccccgccttcagaagaggttttcaggaggaagcgatggctt

SEQ ID NO: 131 - DNA, 40 bp, Homo sapiens
tcattgagtcccccgccttcagaagaggaagcgatggctt

SEQ ID NO: 132 - DNA, 95 bp, Homo sapiens
gcggggtatgaaagtgtagactctcccacgggcagtgaaaactctctcacacaccaatcaaatgacacagactccagtcatgaccctcaagagca

SEQ ID NO: 133 - DNA, 8 bp, Homo sapiens
gcggggca

SEQ ID NO: 134 - DNA, 37 bp, Homo sapiens
gcggggcacactaccgtcggaatgaccctcaagagca

SEQ ID NO: 135 - DNA, 8 bp, Homo sapiens
gcggagca

SEQ ID NO: 136 - DNA, 32 bp, Homo sapiens
gcggggcacactaccgttgaccctcaagagca

SEQ ID NO: 137 - DNA, 36 bp, Homo sapiens
gcggggcacactaccgtcggatgaccctcaagagca

SEQ ID NO: 138 - DNA, 99 bp, Homo sapiens
tgctcttgagggtcatgactggagtctgtgtcatttgattggtgtgtgagagagttttcactgcccgtgggagagtctacactttcataccccgcactg

SEQ ID NO: 139 - DNA, 12 bp, Homo sapiens
tgccccgcactg

SEQ ID NO: 140 - DNA, 12 bp, Homo sapiens
tgctccgcactg

SEQ ID NO: 141 - DNA, 17 bp, Homo sapiens
tgctcttgagggcactg

SEQ ID NO: 142 - DNA, 16 bp, Homo sapiens
tgctcttgaggcactg

SEQ ID NO: 143 - RNA, 165 nt, Artificial
  Cas-beta2 sgRNA targeted RUNX1 site 1 (20 nt spacer)
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaggccuucagaagagggugcau

SEQ ID NO: 144 - RNA, 165 nt, Artificial
  Cas-beta2 sgRNA targeted WTAP site 6 (20 nt spacer)
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaagacgggcagugaaaacucucu

SEQ ID NO: 145 - RNA, 165 nt, Artificial
  Cas-beta2 sgRNA targeted WTAP site 7 (20 nt spacer)
guuaggcguuccgucucgacuaugccguaccacuagaccgagccuacacggcacgcggucauagcguuaaccaaggcguggugacaagccucuuucaggcgucggacacuuaagagcguugaaaaacgcucuuagggaaugaaaggugggagagucuacacuuuc

SEQ ID NO: 146 - RNA, 184 nt, Artificial
  Cas-beta3 sgRNA targeted WTAP site 7 (20 nt spacer)
guuugaaugugcucugccgaagacgccgcacggagccugggccggaaucguagaucgaacgcggcaucgaagcccugcagcccuucggggccaaggcggcgcagcaagccucuuucaggcggcagaguccuuuagagugugaaaacacucuaaaggaaugaaaggugggagagucuacacuuuc

SEQ ID NO: 147 - DNA, 62 bp, Zea mays
aggcagatcctcggccccgcctggaacgccgtgaacttgaacggcgacgcgttgcggaacgc

SEQ ID NO: 148 - DNA, 60 bp, Zea mays
aggcagatcctcggccccgcctggaacgccgtgaacttacggcgacgcgttgcggaacgc

SEQ ID NO: 149 - DNA, 62 bp, Zea mays
gggtcagaggccctcctagcgcagagcatcaagctcgtggacgagttcacctacagcgtgat

SEQ ID NO: 150 - DNA, 37 bp, Zea mays
gggtcagaggccctcctagcgtcacctacagcgtgat

SEQ ID NO: 151 - DNA, 43 bp, Artificial
  Cas-beta2 native spacer 1 target
cccacgggcagtatcggagggcgcgggagaggtgccagccttc

SEQ ID NO: 152 - DNA, 40 bp, Artificial
  Cas-beta2 native spacer 2 target
ccccgttgcaggcaatgatctctgcaactggccgtcgaat

SEQ ID NO: 153 - DNA, 38 bp, Artificial
  Cas-beta2 native spacer 3 target
cccggagtgggtgcgacttaaagcgtttgacggtggct

SEQ ID NO: 154 - protein, 867 aa, Artificial
  D528A cleavage inactive Cas-beta2
MGKNRSSSSDLSPLERSLRKVGENRLERLRVREEKIRKHIEQHPRGKNDHQALHFLLHQIEVERNDLYRNLKDPEYVPKPAKQRRERRQINVAKPPTRPKKEKGPQPESTKYVIRPPVPGKNLPAFASKYEARDTRDDSYQDGRSWTSAPYVEVELPILGADKVIQKLMKFVQKDERSIVRDWATKTYSSIEAAREALLVGAQVSEDVSVWRGLLAETKNAQNFAALSDDQIEAAMSKEAKGADLRPRRAALLVAQRHWVDQTVKAIKESAPSGVDKDTLDRRLRAGLRGFHTAANSGKHTNPQFPYLTAEKPVVPMESVVQSVLAFLDDPDDQRYTKDKEDDKKRHRVTVLQKELGKARPRKRLELQTPKWAGRPTVKGTISKRRDAALVWDTSKEANGLCLALPIGGMPKIDVEQFIYQDGTSLLSDCQIASKTTKKGAACAVLPLKPKHDFLRWFTKHVENHNPDAPLERRCLHNTTQFVIVDPEGPRPRLFVRPVFKFYDPGKTVPNTHETWKKPDCRYLVGIARGINYVLRAVVVDTEEKKVIADIGLPGRKHEWRMIRDEIAYHQQMRDLARNTGKHASVVAKHVRALALARKKDRALGKFATVEAVAELVKKCEQDYGSGNYCFVLEDLDMGAMNLKRNNRVKHMAVMEEALVNQMRKQGYAYDGRRGRVDGVRHEGAWYTSQVSPFGWWAKRDEVEEAWKRDKTRPIGRKVGNWYEMPEPGQDGDRPDTYRKGYWSKPKNAEGKPYGRNRFSVEPGDEKPDAERRFCWGSELFWDPNVKSFKGKEFPEGVVLDADFVGALNIALRPLVNDGQGKGFKAEDMAREHTILNPQFKIACQIPVYEFVEEDGDKWAALRRIML

SEQ ID NO: 155 - protein, 867 aa, Artificial
  E634A cleavage inactive Cas-beta2
MGKNRSSSSDLSPLERSLRKVGENRLERLRVREEKIRKHIEQHPRGKNDHQALHFLLHQIEVERNDLYRNLKDPEYVPKPAKQRRERRQINVAKPPTRPKKEKGPQPESTKYVIRPPVPGKNLPAFASKYEARDTRDDSYQDGRSWTSAPYVEVELPILGADKVIQKLMKFVQKDERSIVRDWATKTYSSIEAAREALLVGAQVSEDVSVWRGLLAETKNAQNFAALSDDQIEAAMSKEAKGADLRPRRAALLVAQRHWVDQTVKAIKESAPSGVDKDTLDRRLRAGLRGFHTAANSGKHTNPQFPYLTAEKPVVPMESVVQSVLAFLDDPDDQRYTKDKEDDKKRHRVTVLQKELGKARPRKRLELQTPKWAGRPTVKGTISKRRDAALVWDTSKEANGLCLALPIGGMPKIDVEQFIYQDGTSLLSDCQIASKTTKKGAACAVLPLKPKHDFLRWFTKHVENHNPDAPLERRCLHNTTQFVIVDPEGPRPRLFVRPVFKFYDPGKTVPNTHETWKKPDCRYLVGIDRGINYVLRAVVVDTEEKKVIADIGLPGRKHEWRMIRDEIAYHQQMRDLARNTGKHASVVAKHVRALALARKKDRALGKFATVEAVAELVKKCEQDYGSGNYCFVLADLDMGAMNLKRNNRVKHMAVMEEALVNQMRKQGYAYDGRRGRVDGVRHEGAWYTSQVSPFGWWAKRDEVEEAWKRDKTRPIGRKVGNWYEMPEPGQDGDRPDTYRKGYWSKPKNAEGKPYGRNRFSVEPGDEKPDAERRFCWGSELFWDPNVKSFKGKEFPEGVVLDADFVGALNIALRPLVNDGQGKGFKAEDMAREHTILNPQFKIACQIPVYEFVEEDGDKWAALRRIML